(19) United States
(12) Patent Application Publication
(10) Pub. No.: US 2026/0188466 A1
(43) Pub. Date: Jul. 2, 2026

(54) METHODS AND SYSTEMS OF FACILITATING CULTURALLY-AUTHENTIC AND NON-REPETITIVE MEAL PLANNING

(71) Applicants: Amruta Abhay Pokarna, Bothell, WA (US); Aditya Lodha, Bothell, WA (US)

(72) Inventors: Amruta Abhay Pokarna, Bothell, WA (US); Aditya Lodha, Bothell, WA (US)

(21) Appl. No.: 19/550,292

(22) Filed: Feb. 26, 2026

Related U.S. Application Data
(60) Provisional application No. 63/946,229, filed on Dec. 22, 2025.

(51) Int. Cl. G16H 20/60

(52) U.S. Cl. CPC G16H 20/60

(57) ABSTRACT

A method of facilitating culturally-authentic and non-repetitive meal planning. The method includes receiving a meal planning request data, receiving a user meal history data, identifying a candidate meal data set, selecting a candidate meal data from the candidate meal data set, validating the candidate meal data against a cuisine signature data by executing a tri-level validation, rejecting the candidate meal data when the tri-level validation fails, classifying the candidate meal data as an authentic candidate meal data when the tri-level validation passes, and repeating the selecting, validating, rejecting, and classifying for each day of the multi-day planning duration.

### REFERENCE TO RELATED APPLICATIONS

This application claims the benefit of U.S. Provisional Patent Application No. 63/946,229, titled “METHODS AND SYSTEMS FOR PROVIDING A MEAL PLAN SUGGESTION TO A USER”, filed Dec. 22, 2025, which is incorporated by reference herein in its entirety.

### FIELD OF DISCLOSURE

The present disclosure relates to the field of data processing. More specifically, the present disclosure relates to methods and systems of facilitating culturally-authentic and non-repetitive meal planning.

### BACKGROUND

The present disclosure relates generally to computer-implemented systems and methods for automated meal planning, nutritional recommendation, and food selection implemented using networked computing platforms. Such systems are commonly deployed in software-as-a-service environments to generate meal plans and food recommendations based on user-specific inputs such as dietary preferences, nutritional targets, and cultural considerations. With the increasing reliance on digital platforms for health, wellness, and food management, the technical effectiveness of these systems has become an important consideration.

In the given field, a desirable aspect is to achieve the objective of generating meal plans that maintain diversity over time while satisfying multiple constraints, including nutritional requirements, dietary restrictions, and cultural relevance. In many use cases, meal planning systems are expected to generate plans spanning multiple days or recurring planning cycles, which requires the system to manage historical meal data, reduce unnecessary repetition, and adapt outputs over time. Achieving the said objective in a scalable and automated manner presents a number of technical challenges.

Various existing systems have attempted to address aspects of the said objective. For example, an existing system generates meal plans by scoring candidate recipes against a range of user-defined preferences, nutritional constraints, and contextual factors. Similarly, another existing system discloses techniques for generating nutrition schedules using optimization algorithms that take into account health, nutrient, and cultural constraints. Other existing approaches focus on substitution and adjustment of meal components, including systems that allow substitution of food items while preserving nutritional targets and dietary restrictions, and techniques to select replacement ingredients based on availability, cooking constraints, and similarity metrics.

Further, some existing systems attempt to encourage meal variety or compatibility by describing a recipe recommendation engine that considers compatibility of dishes within a meal and may avoid redundant ingredient combinations, and other existing systems describe generating nutritional catering information while applying similarity constraints to avoid highly similar recipes appearing in close temporal proximity.

While the existing systems, technologies, and studies address various aspects of meal planning, nutrition optimization, ingredient substitution, culinary compatibility, and authenticity analysis, they do not fully resolve challenges associated with generating non-repetitive meal plans across multiple planning cycles, managing historical meal usage in a structured manner, coordinating ingredient reuse over time, or maintaining consistent cultural relevance at finer levels of granularity. As a result, limitations remain in achieving scalable, automated meal planning that balances diversity, historical context, and multiple concurrent constraints.

Therefore, there is a need for improved methods and systems of facilitating culturally-authentic and non-repetitive meal planning that may overcome one or more of the preceding problems.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method of facilitating culturally-authentic and non-repetitive meal planning. Further, the method may include receiving, using a communication device, a meal planning request data associated with a user from a client device. Further, the meal planning request data specifies a geographic sub-regional cuisine identity and a multi-day planning duration. Further, the method may include receiving, using the communication device, a user meal history data associated with the user from the client device. Further, the method may include identifying, using a processing device, a candidate meal data set based on the meal planning request data. Further, the method may include selecting, using the processing device, a candidate meal data from the candidate meal data set. Further, the candidate meal data may include a candidate ingredient data, a candidate nutritional data, and a candidate preparation instruction data. Further, the method may include validating, using the processing device, the candidate meal data against a cuisine signature data corresponding to the geographic sub-regional cuisine identity by executing a tri-level validation. Further, the tri-level validation may include determining an absence of each of one or more prohibited ingredients in the candidate ingredient data. Further, the one or more prohibited ingredients are defined by the cuisine signature data. Further, the tri-level validation may include determining a presence of each of one or more mandatory ingredients in the candidate ingredient data. Further, the one or more mandatory ingredients are defined by the cuisine signature data. Further, the tri-level validation may include calculating a cuisine signature score based on a weighted characteristic ingredient data derived from the candidate ingredient data and comparing the cuisine signature score to a configurable authenticity threshold. Further, the method may include rejecting, using the processing device, the candidate meal data when the tri-level validation fails. Further, the tri-level validation fails when at least one of the one or more prohibited ingredients is determined to be present in the candidate ingredient data, when at least one of the one or more mandatory ingredients is determined to be absent from the candidate ingredient data, or when the cuisine signature score is determined to be below the configurable authenticity threshold. Further, the method may include classifying, using the processing device, the candidate meal data as an authentic candidate meal data when the tri-level validation passes. Further, the method may include repeating, using the processing device, the selecting, the validating, the rejecting, and the classifying for each day of the multi-day planning duration to obtain a plurality of authentic candidate meal data, each of the plurality of authentic candidate meal data being associated with a respective day of the multi-day planning duration.

The present disclosure provides a system of facilitating culturally-authentic and non-repetitive meal planning. Further, the system may include a communication device configured for receiving a meal planning request data associated with a user from a client device. Further, the meal planning request data specifies a geographic sub-regional cuisine identity and a multi-day planning duration. Further, the communication device may be configured for receiving a user meal history data associated with the user from the client device. Further, the system may include a processing device communicatively coupled with the communication device. Further, the processing device may be configured for identifying a candidate meal data set based on the meal planning request data. Further, the processing device may be configured for selecting a candidate meal data from the candidate meal data set. Further, the candidate meal data comprises a candidate ingredient data, a candidate nutritional data, and a candidate preparation instruction data. Further, the processing device may be configured for validating the candidate meal data against a cuisine signature data corresponding to the geographic sub-regional cuisine identity by executing a tri-level validation. Further, the tri-level validation may include determining an absence of each of one or more prohibited ingredients in the candidate ingredient data. Further, the one or more prohibited ingredients are defined by the cuisine signature data. Further, the tri-level validation may include determining a presence of each of one or more mandatory ingredients in the candidate ingredient data. Further, the one or more mandatory ingredients are defined by the cuisine signature data. Further, the tri-level validation may include calculating a cuisine signature score based on a weighted characteristic ingredient data derived from the candidate ingredient data and comparing the cuisine signature score to a configurable authenticity threshold. Further, the processing device may be configured for rejecting the candidate meal data when the tri-level validation fails. Further, the tri-level validation fails when at least one of the one or more prohibited ingredients is determined to be present in the candidate ingredient data, when at least one of the one or more mandatory ingredients is determined to be absent from the candidate ingredient data, or when the cuisine signature score is determined to be below the configurable authenticity threshold. Further, the processing device may be configured for classifying the candidate meal data as an authentic candidate meal data when the tri-level validation passes. Further, the processing device may be configured for repeating the selecting, the validating, the rejecting, and the classifying for each day of the multi-day planning duration to obtain a plurality of authentic candidate meal data, each of the plurality of authentic candidate meal data being associated with a respective day of the multi-day planning duration.

The present disclosure provides a non-transitory computer-readable medium storing instructions that, when executed by one or more processors of an online server, cause the online server to perform operations for facilitating culturally-authentic and non-repetitive meal planning. Further, the operations may include receiving a meal planning request data associated with a user from a client device. Further, the meal planning request data specifies a geographic sub-regional cuisine identity and a multi-day planning duration. Further, the operations may include receiving a user meal history data associated with the user from the client device. Further, the operations may include identifying a candidate meal data set based on the meal planning request data. Further, the operations may include selecting a candidate meal data from the candidate meal data set. Further, the candidate meal data comprises a candidate ingredient data, a candidate nutritional data, and a candidate preparation instruction data. Further, the operations may include validating the candidate meal data against a cuisine signature data corresponding to the geographic sub-regional cuisine identity by executing a tri-level validation. Further, the tri-level validation may include determining an absence of each of one or more prohibited ingredients in the candidate ingredient data. Further, the one or more prohibited ingredients are defined by the cuisine signature data. Further, the tri-level validation may include determining a presence of each of one or more mandatory ingredients in the candidate ingredient data. Further, the one or more mandatory ingredients are defined by the cuisine signature data. Further, the tri-level validation may include calculating a cuisine signature score based on a weighted characteristic ingredient data derived from the candidate ingredient data and comparing the cuisine signature score to a configurable authenticity threshold. Further, the operations may include rejecting the candidate meal data when the tri-level validation fails. Further, the tri-level validation fails when at least one of the one or more prohibited ingredients is determined to be present in the candidate ingredient data, when at least one of the one or more mandatory ingredients is determined to be absent from the candidate ingredient data, or when the cuisine signature score is determined to be below the configurable authenticity threshold. Further, the operations may include classifying the candidate meal data as an authentic candidate meal data when the tri-level validation passes. Further, the operations may include repeating the selecting, the validating, the rejecting, and the classifying for each day of the multi-day planning duration to obtain a plurality of authentic candidate meal data, each of the plurality of authentic candidate meal data being associated with a respective day of the multi-day planning duration.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

The drawings presented with this disclosure may illustrate representative and non-limiting arrangements of hardware components, software modules, artificial intelligence subsystems, machine learning architectures, data processing pipelines, user interfaces, network topologies, and memory arrangements that may be used to understand embodiments of the present subject matter. They may depict functional or conceptual layouts intended to facilitate the explanation of the disclosed principles. The geometric appearance, dimensional proportions, ordering, grouping, and naming of elements within the drawings are not intended to imply any restriction on implementation. The drawings may schematically portray computing environments containing client devices, servers, distributed computing clusters, communication networks, storage systems, or artificial intelligence models arranged for training, inference, or combined operations. The drawings may include simplified symbolic representations of algorithmic processes, workflows, blocks, or modules; such symbolic representations are treated as abstractions of underlying hardware and software operations rather than literal structural requirements. Similarly, lines connecting components may represent logical associations, communication pathways, or data relationships rather than any specific physical wiring or layout. These figures may also illustrate non-exhaustive examples of operational stages, sequencing, or interactions among artificial intelligence components such as encoders, decoders, generators, discriminators, featurizers, transformers, or safety-validation modules. Any specific combination or configuration shown is presented for explanatory clarity only. Additional drawings, alternative views, or more granular depictions may be used without affecting the scope of the claims.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 is a block diagram of a machine-learning system 300 for implementing various embodiments of this disclosure, in accordance with some embodiments.

FIG. 4 illustrates a flowchart of a method 400 for providing a meal plan suggestion to a user, in accordance with some embodiments.

FIG. 5 illustrates a flowchart of a method 500 for providing a meal plan suggestion to a user including generating, using the processing device 1104, a validation result data, in accordance with some embodiments.

FIG. 6 illustrates a flowchart of a method 600 for providing a meal plan suggestion to a user including generating, using the processing device 1104, an updated requirement data, in accordance with some embodiments.

FIG. 7 illustrates a flowchart of a method 700 for providing a meal plan suggestion to a user including generating, using the processing device 1104, a modified third meal plan, in accordance with some embodiments.

FIG. 8 illustrates a flowchart of a method 800 for providing a meal plan suggestion to a user including assigning, using the processing device 1104, the identifier to each of the plurality of ingredients associated with the sample meal plan, in accordance with some embodiments.

FIG. 9 illustrates a flowchart of a method 900 for providing a meal plan suggestion to a user including analyzing, using the processing device 1104, the historical meal plan data, in accordance with some embodiments.

FIG. 10 illustrates a flowchart of a method 1000 for providing a meal plan suggestion to a user including generating, using the processing device 1104, a modified sample meal plan relative to the meal replacement request data, in accordance with some embodiments.

FIG. 11 illustrates a block diagram of a system 1100 for providing a meal plan suggestion to a user, in accordance with some embodiments.

FIG. 12A illustrates a tri-level validation process for sub-regional cuisine fingerprinting, in accordance with some embodiments.

FIG. 12B illustrates a continuation of the tri-level validation process for sub-regional cuisine fingerprinting, in accordance with some embodiments.

FIG. 13 illustrates a forward-chaining leftover optimization algorithm, in accordance with some embodiments.

FIG. 14A illustrates a constraint-preserving bounded swap algorithm with intra-plan deduplication, in accordance with some embodiments.

FIG. 14B illustrates a continuation of the constraint-preserving bounded swap algorithm with intra-plan deduplication, in accordance with some embodiments.

FIG. 15A illustrates a historical meal deduplication flow with content-based fingerprinting and retry logic, in accordance with some embodiments.

FIG. 15B illustrates a continuation of the historical meal deduplication flow with content-based fingerprinting and retry logic, in accordance with some embodiments.

FIG. 16A illustrates a flowchart of a method 1600 of facilitating culturally-authentic and non-repetitive meal planning, in accordance with some embodiments.

FIG. 16B illustrates a continuation of the flowchart of the method 1600 of facilitating culturally-authentic and non-repetitive meal planning, in accordance with some embodiments.

FIG. 17 illustrates a flowchart of a method 1700 of facilitating the culturally-authentic and non-repetitive meal planning, in accordance with some embodiments.

FIG. 18A illustrates a flowchart of a method 1800 of facilitating the culturally-authentic and non-repetitive meal planning, in accordance with some embodiments.

FIG. 18B illustrates a continuation of the flowchart of the method 1800 of facilitating the culturally-authentic and non-repetitive meal planning, in accordance with some embodiments.

FIG. 19 illustrates a block diagram of a system 1900 of facilitating culturally-authentic and non-repetitive meal planning, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denote “at least one,” and/or “one or more,” but do not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

The detailed description that follows may provide a framework for describing computer-implemented systems, artificial intelligence systems, distributed learning infrastructures, data processing pipelines, and hardware and software arrangements suitable for implementing embodiments shown in the drawings. Terms such as processing, computing, determining, or generating refer to actions performed by computing systems, computing devices, or electronic devices that manipulate data represented as physical signals, stored values, or encoded information within registers, memory structures, and storage devices.

The present disclosure contemplates implementations involving artificial intelligence, machine learning, distributed computation, and computer-implemented systems operating upon data represented as physical electronic or optical signals. Descriptions of processing, analyzing, determining, transforming, encoding, decoding, generating, inferring, synthesizing, modifying, storing, retrieving, ranking, filtering, validating, classifying, or otherwise manipulating information are to be understood as referring to the actions of computing systems, computing devices, electronic devices, or computational circuits that manipulate such signals in memory elements, registers, buffers, or storage media.

The disclosure contemplates implementations in which artificial intelligence systems perform perception, synthesis, inference, prediction, or generation of information and/or data using models whose configurations may evolve based on training, feedback, or adaptive learning processes. A model may initially be configured with a set of parameters and architectural structures that define its behavior, and this configuration may change automatically as the model encounters training inputs, validation data, reference data, or instructor-provided feedback. The model may modify its internal state through optimization techniques, gradient updates, reinforcement signals, vector transformations, attention mechanisms, latent variable adjustments, embedding refinements, or other learning operations executed electronically. Such modifications may occur over extended cycles, partial cycles, or continual learning sequences without explicit intervention by a human.

The disclosure contemplates systems involving data ingestion pipelines that gather input from sources including but not limited to sensor signals, event streams, text data, image data, audio data, video data, structured and unstructured repositories, application logs, telemetric feeds, network services, or human-generated content. Ingestion functions may include filtering, normalization, augmentation, segmentation, batching, tokenization, windowing, compression, encryption, decryption, hashing, deduplication, contextualization, and mapping to internal formats. Intermediate components may transform this data into derived representations, including embeddings, latent encodings, feature tensors, multi-modal joint representations, or contextual vectors suitable for use by downstream modeling engines. These transformations may be performed using neural networks, statistical encoders, dimensionality-reduction algorithms, or hybrid computational modules.

The disclosure contemplates machine learning systems that may employ advanced architectures such as transformer networks, encoder-decoder stacks, mixture-of-experts structures, diffusion models, recurrent networks, convolutional hierarchies, attention-based models, retrieval-augmented architectures, cross-modal alignment engines, graph neural networks, probabilistic models, auto-encoding frameworks, or hybrid symbolic-neural systems. Such models may implement deep layers configured to perform operations including attention calculations, feed-forward projections, gating operations, positional encoding, normalization steps, multi-head routing, sequential decoding, or latent pathway selection. Multi-modal systems may combine textual, visual, auditory, sensory, or structured inputs within joint representational spaces. Embeddings may be learned from large corpora or multi-modal datasets and may encode semantic, syntactic, structural, temporal, spatial, or contextual relationships across modalities. These embeddings may be dynamically updated as the system encounters new information, thereby improving consistency, expressiveness, or alignment with real-world contexts.

The disclosure contemplates training processes that may involve supervised learning, unsupervised learning, semi-supervised learning, self-supervised learning, reinforcement learning, preference optimization, curriculum-based learning, active learning, or continual learning. Training operations may include forward passes through the model, backward propagation of gradients, update steps using optimization algorithms, adaptive learning-rate scheduling, regularization steps, loss-function evaluation, and checkpointing of intermediate states. Training datasets may include real-world data, synthetic data, simulated data, augmented data, or mixtures thereof. Validation procedures may evaluate performance metrics, generalization behavior, safety constraints, or compliance with domain-specific criteria. In some implementations, refinement cycles may incorporate human-in-the-loop interventions, reward model shaping, safety evaluator feedback, or guided corrections.

The disclosure contemplates distributed or federated execution in which computation is partitioned across multiple hardware devices, regions, or clusters. Certain operations may occur at edge devices for low latency, while others may be delegated to remote servers, cloud clusters, datacenters, or specialized compute fabrics. Components may communicate over wired or wireless networks supporting data exchange, synchronization, replication, or model-state updates. Distributed learning processes may synchronize gradients, coordinate model versions, merge updates across shards, or exchange activation values within parallel training regimes. Distributed inference may involve routing requests across replicas, balancing load through orchestration layers, or selecting model pathways dynamically. Network connections may include encryption, authentication, secure session management, or routing protocols appropriate for maintaining privacy, integrity, or availability.

The disclosure contemplates orchestration layers capable of managing complex workflows involving model invocation, tool invocation, external data retrieval, decision routing, fallback selection, multi-model aggregation, post-processing evaluation, or safety governance. Orchestration environments may evaluate contextual signals, metadata, user characteristics, or policy constraints to determine which models, subsystems, or computational branches should be executed. Such environments may dynamically alter execution pathways based on estimated performance, resource availability, model confidence, safety risk, or real-time system health. Post-processing components may evaluate generated outputs for compliance with content policies, statutory requirements, operational constraints, or domain-specific decision rules.

The disclosure contemplates safety-oriented components that evaluate model outputs or intermediate representations for consistency with safety criteria, quality thresholds, regulatory considerations, factual accuracy constraints, domain restrictions, or alignment requirements. Safety modules may employ auxiliary models, discriminators, rule sets, statistical detectors, confidence estimators, or hybrid evaluators to identify undesirable outputs. These modules may trigger remediation actions, including output modification, output rejection, re-routing through alternate inference pathways, invocation of corrective models, or escalation for human review. Safety processes may incorporate real-time validation, contextual scoring, adversarial robustness analysis, anomaly detection, or controlled generation constraints.

The disclosure contemplates governance structures including policy managers, audit loggers, compliance trackers, version controllers, and provenance systems that associate model outputs with contextual metadata, historical signals, update events, training sources, or safety evaluations. Systems may maintain lineage records documenting which model version, configuration state, or training dataset contributed to an outcome. Governance modules may ensure that system behavior aligns with formal requirements such as fairness principles, legal obligations, industry standards, or institutional guidelines.

The disclosure contemplates storage and memory systems capable of storing model parameters, datasets, embeddings, logs, metrics, checkpoints, execution traces, and auxiliary information used to configure or interpret model behavior. The computing device may include these storage systems. Storage media may contain instructions, configurations, or data structures that, when accessed by the computing device, configure that device to carry out the operations described herein. Such media may include executables, bytecode, machine code, firmware, microcode, program modules, configuration files, architectural descriptors, or schema definitions.

The disclosure contemplates user interfaces that permit human operators to view model outputs, initiate tasks, modify configurations, inspect metrics, interact with logs, evaluate safety signals, or guide system adaptation. Interfaces may be multimodal and may support textual input, speech commands, visual interaction, gesture control, or programmatic invocation through application programming interfaces (APIs). Administrative interfaces may allow for reviewing system performance, tuning operational thresholds, enabling or disabling features, monitoring resource use, examining generated content, or initiating refinement workflows.

The disclosure contemplates systems in which instructions are executed entirely on a single device, partially on multiple devices, or cooperatively across remote and local environments. Code may execute directly on hardware, within firmware, inside virtual machines, inside containers, or through any combination of software and hardware interactions. Computational instructions may be stored locally, transferred via communication networks, or streamed from remote systems.

Interpretation of terms in this disclosure is governed by principles commonly applied by persons of ordinary skill in the relevant field. Technical and scientific terms used herein should be understood in a manner consistent with their usage in the field of artificial intelligence, machine learning, computing, networking, data storage, or any related discipline. Terms describing functionality should not be interpreted as strictly structural unless explicitly stated. Phrases such as configured to, adapted to, operable to, or capable of, indicate permissible functionality rather than structural limitations. Terms such as “a” or “an” encompass one or more unless clearly contradicted by context. Terms joined by or should be interpreted as inclusive, and terms joined by and should be interpreted as collective.

The description set forth herein provides a broad and flexible framework intended to support a wide range of computer-implemented, machine-learning-enabled, distributed, and multimodal embodiments. Variations may include reallocation of tasks, substitution of algorithms, reconfiguration of models, changes to pipeline ordering, or adoption of alternate hardware. No combination or arrangement mentioned herein should be regarded as required unless explicitly stated.

The present disclosure contemplates implementations that employ advanced mathematical frameworks characteristic of modern artificial intelligence systems. Machine learning models may be conceptualized as parameterized functions that map elements of an input space to elements of an output space. Such a function may be defined over real-valued, complex-valued, vector-valued, tensor-valued, or mixed-modal domains. The machine learning models may implement successive transformations applied to an ordered set of input vectors using compositions of linear operators, nonlinear activations, attention functions, normalization operations, and dimensional projections.

Model parameters may be represented as ordered collections of real-valued scalars arranged into structures such as matrices, tensors, kernels, filters, or embeddings. These parameters may be optimized by minimizing a loss functional defined over an expected distribution of input-output pairs. The optimization process may involve computing gradients of the loss functional with respect to each model parameter, followed by an update step that serves to reduce the value of the loss functional. Gradient computation may use automatic differentiation frameworks that symbolically or numerically propagate partial derivatives backward through a computational graph.

Attention mechanisms may employ a similarity measure between projected query vectors and projected key vectors. This similarity measure may yield a weight distribution over contextual elements. The weighted combination of projected value vectors may form an attention output that is subsequently transformed through additional layers. Multiple independent attention heads may be aggregated to capture heterogeneous relationships within the input domain. Cross-attention mechanisms may operate similarly but with distinct source and target sequences.

Normalization steps may rescale intermediate representations using learned scaling and shifting coefficients. Activation functions may introduce nonlinearity by applying element-wise transformations selected to ensure differentiability and expressive capacity. Residual pathways may combine transformed and untransformed representations to facilitate stable gradient propagation under deep compositions. Positional encodings or structural embeddings may inject ordering, spatial, temporal, or relational information into otherwise permutation-invariant architectures.

Multi-modal models may operate over domains that combine text, image, audio, video, sensor, or structured signals. These domains may be embedded into a common vector space through learned projection operators. Joint training processes may enforce alignment constraints that minimize representational divergence between modalities while preserving intra-modal semantics.

Diffusion frameworks may model data generation as the reversal of a stochastic corruption process. A forward process may incrementally add noise to data samples, while a learned reverse process may approximate the time-reversed conditional probability distribution. The reverse process may be parameterized by a neural network trained to denoise intermediate states. Continuous-time formulations may model this process using stochastic differential equations whose drift and diffusion terms are learned through score-matching or related techniques.

Reinforcement-based procedures may model learning as an optimization of expected reward under a policy function. The policy may produce distributions over actions given a latent or explicit representation of the environment state. Policy gradients may be estimated from sampled trajectories, and advantage estimators may reduce the variance of such gradients. Value functions may approximate the expected cumulative reward, and these approximations may be updated through temporal-difference learning.

Generative models may be expressed in probabilistic terms as joint or conditional distributions parameterized by neural architectures. Such models may perform sampling by iteratively drawing latent variables from a learned distribution and transforming those variables into output space. Variational models may introduce auxiliary latent variables whose posterior distributions are approximated through recognition functions that optimize an evidence-bound objective.

Matrix decompositions, spectral analysis, manifold learning, kernel operators, and other mathematical constructs may be incorporated to improve expressiveness, stability, or computational efficiency. Training may involve sophisticated schedulers, trust-region constraints, adaptive learning-rate schemes, gradient-norm clipping, regularization penalties, entropy maximization, attention masking, or mixed-precision arithmetic.

All such mathematical constructs are conceptual, descriptive, and non-limiting. The disclosure encompasses any differentiable or non-differentiable optimization method, any discrete or continuous learning paradigm, and any representational transformation that may be understood by a person of ordinary skill in the field.

Further, the disclosure provides a computing environment that may include a combination of client devices, servers, distributed computing clusters, databases, external data sources, network nodes, and interface endpoints. Further, the computing environment may support artificial intelligence workloads, including perception, synthesis, inference, prediction, and generation, using hardware and software foundations designed for high-throughput and low-latency operation. Embodiments may involve the coordinated use of multiple machine learning models, whose configurations may evolve over time as they learn from training, validation, reference, or feedback data. Machine learning models may adjust their internal parameters through supervised, unsupervised, or reinforcement-based processes, allowing automatic electronic improvements to their performance based on input data and observed outcomes.

Further, the disclosure provides a machine-learning architecture that may include engines or modules such as a data input engine, a data retrieval engine, a data transform engine, a featurization engine, a modeling engine, a generative engine, a validation engine, a feedback engine, and a refinement engine. A data input pipeline may obtain structured or unstructured information from various sources, transform the information into model-compatible forms, and store such transformed data in memory or storage accessible to downstream components. A modeling engine may perform tasks such as model training, re-configuration, validation, and testing, executing iterative processes across multiple cycles or passes through training data. A predictive or generative engine may construct outputs based on intermediate representations, learned embeddings, or latent encodings generated by layers such as encoder-decoder structures, attention mechanisms, or multi-layer transformer architectures. Embeddings may represent discrete entities such as words, documents, or images as continuous vectors in high-dimensional spaces, capturing semantic or structural relationships useful for downstream tasks.

Further, the disclosure may provide a distributed or cloud-based operation which may include multiple physical or virtual instances of computing devices, distributed across data centers or network boundaries. Functions may be partitioned across machines to achieve parallelism, redundancy, fault tolerance, or improved throughput. Distributed systems may use load balancing mechanisms to maintain stable processing, memory, or bandwidth utilization across clusters and avoid overload conditions. Such deployments may require communication over wired or wireless networks that implement a variety of protocols, including HTTP, HTTPS, MQTT, CoAP, or any other suitable communication framework. Communication channels may include local networks, wide-area networks, personal-area networks, or global communication systems, potentially utilizing secure, encrypted sessions such as SSL-based channels.

Further, the disclosure may provide an algorithm, process, or flow diagram that may include operations that may occur in sequences, reversed orders, concurrently, or in partially overlapping timelines, depending on the implementation. Blocks representing actions in a flowchart may correspond to program modules, instruction sequences, or hardware logic capable of performing the specified acts. Such operations may manipulate physical quantities such as electrical or magnetic signals stored or transferred among memory units, registers, storage devices, or communication media. Flow diagrams may be realized through software running on general-purpose processors, through dedicated hardware circuits, or through combinations of both.

Further, the disclosure may provide a user interface which may include graphical displays, dashboards, selection controls, input fields, monitoring elements, or multimodal interaction surfaces, allowing users to interact with computing systems in speech, touch, gesture, or other modalities. Such interfaces may be presented through client devices, server applications, or remote access platforms and may support visualization of model behavior, system performance, or configuration parameters.

Further, the described features may be combined, rearranged, omitted, or substituted without departing from the principles disclosed. Variations may involve distributing functionality across devices, merging components, implementing features in hardware rather than software, or employing alternative communication protocols. Many such variations and modifications are intended to fall within the scope of the disclosure as understood by persons skilled in the art.

The detailed description of the drawings, therefore, provides a foundation for describing technical, architectural, and operational aspects of embodiments, while allowing broad flexibility in how such embodiments may be implemented in practice. The scope of such embodiments is governed by the claims rather than the illustrative content of the drawings.

In some embodiments, a system consistent with this disclosure includes one or more client devices, one or more servers, and one or more data stores coupled by one or more networks. The client devices may include computing platforms equipped with data processing hardware and memory hardware. The servers may include data servers, application servers, web servers, proxy servers, or cloud computing services that provide shared processing, storage, and networking resources. The data stores can include databases, object stores, file systems, or other repositories that persist configuration data, training data, logs, model artifacts, and other information.

The networks can include public and private networks, such as local area networks, wide area networks, and cloud networks, using wired or wireless communication links. The networks can provide routing, addressing, access control, encryption, and related functionality using standard or proprietary protocols.

For purposes of this disclosure, artificial intelligence systems may include arrangements of software and hardware that perform tasks such as perception, prediction, planning, or generation based on input data. These systems can employ one or more models, such as statistical models, neural networks, decision trees, or other machine learning models. As used herein, a “model” can refer to a parameterized function, an ensemble of such functions, or a collection of cooperating components that process data and produce outputs.

In some embodiments, the system includes a data input engine that obtains data from one or more sources, such as application logs, sensor streams, structured databases, and unstructured content. The data input engine can retrieve, filter, aggregate, or transform the data into feature representations suitable for model consumption. Data sources can include training data, validation data, and reference data used to evaluate and calibrate model behavior. Further, the system includes a modeling engine that manages one or more training processes for one or more models. Moreover, the modeling engine can select model architectures, initialize parameters, and apply training algorithms such as supervised learning, semi supervised learning, unsupervised learning, reinforcement learning, or combinations thereof. The modeling engine can also manage hyper parameters, training schedules, and evaluation procedures across epochs or passes through the data. Further, the system may include a generative response engine or an inference engine that receives prompts or other inputs and generates outputs using one or more models. For example, a natural language interface can receive a text prompt, embed or otherwise encode the prompt, process the encoded prompt using a transformer based model or other sequence model, and generate a sequence of tokens that are decoded into an output. The engine can generate multiple candidate outputs and apply validation or ranking logic to select a final result according to quality, safety, or relevance criteria. Further, the system may include a feedback engine that collects explicit or implicit feedback signals, such as user ratings, corrective edits, or outcome metrics derived from downstream tasks. Furthermore, the system may include a refinement engine that uses the feedback to adjust model parameters, routing logic, or policies, for example, by performing additional training steps, updating reward models, or modifying configuration parameters.

The systems described herein can be implemented using centralized, decentralized, or hybrid arrangements. For instance, models may be deployed in cloud environments, on edge devices, or across both, depending on requirements such as latency, privacy, cost, and reliability. Load balancing and resource management components can distribute processing across devices or data centers and can provide elasticity to accommodate changing workloads.

Certain embodiments may expose functionality through application programming interfaces, software development kits, or graphical user interfaces. Client applications can submit requests to backend services, which can apply authentication, authorization, logging, and policy enforcement before invoking models or tools and returning results.

The systems and methods disclosed herein can be implemented in hardware, software, firmware, or any combination thereof. In some embodiments, operations are carried out by one or more processors executing program instructions stored on one or more non-transitory computer readable media. Program instructions, when executed by the processors, cause the processors to perform the operations described herein.

Instructions can be delivered to computing devices in various ways, such as pre-installation, physical distribution of media, or transmission over networks. Instructions received over a network can be stored in memory or persistent storage and then executed by one or more processors. Dedicated hardware logic, such as application-specific integrated circuits or field programmable gate arrays, can be used alone or in combination with software to implement certain functionality.

Any methods described in connection with embodiments of the present disclosure can be represented as one or more flow diagrams or state diagrams. Blocks in such diagrams can correspond to modules, components, operations, or code segments that implement the associated functionality. Blocks can be reordered, combined, executed concurrently, or omitted according to implementation-specific considerations, unless a particular ordering is required by the claims.

Examples and embodiments described herein illustrate, rather than limit, the claimed subject matter. Certain features have been described in connection with particular embodiments for clarity, but other embodiments can include such features in different combinations. Features described in separate embodiments can be combined, and features described in a single embodiment can be separated.

In general, the method(s) disclosed herein may be performed by at least one computing device. Further, the at least one computing device may be and/or may include at least one server (i.e., at least one server computer). Further, the at least one computing device may be and/or may include a communication device, a processing device, and a storage device. For example, in some embodiments, the method may be performed by the at least one server in communication with at least one client device over a communication network such as, for example, the Internet. In some other embodiments, the method may be performed by one or more of at least one server computer, at least one client device, at least one network device, at least one sensor, and at least one actuator. Examples of the at least one client device, and/or the at least one server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on.

Further, in some embodiments, the method may include receiving, using the communication device, at least one input from the client device, wherein the at least one input comprises at least one query and at least one characteristic corresponding to at least one of a capability, a limitation, a state, and a status of the client device. Further, the method may include obtaining, using the processing device, at least one output using at least one large language model based on the at least one input and at least one instruction, wherein the at least one large language model comprises a generative artificial intelligence model. Further, the obtaining of the at least one output may include determining at least one value for at least one parameter associated with the at least one large language model based on the at least one instruction, wherein the at least one parameter governs a behavior of the at least one large language model. Further, the at least one parameter comprises one or more hyperparameters, one or more model parameters, etc., of the at least one large language model. Further, the obtaining of the at least one output may include inputting the at least one input to the at least one large language model based on the determining. Further, the at least one large language model is configured for generating the at least one output based on the at least one value of the at least one parameter and the at least one input. Further, the at least one output comprises at least one of a candidate meal data and, when duplication is identified, a replacement candidate meal data, wherein the at least one of the candidate meal data and the replacement candidate meal data comprises a candidate ingredient data, a candidate nutritional data, and a candidate preparation instruction data. Further, the obtaining of the at least one output may include normalizing, using the processing device, the candidate ingredient data, the candidate nutritional data, and the candidate preparation instruction data to obtain a normalized candidate ingredient data, a normalized candidate nutritional data, and a normalized candidate preparation instruction data, and forming a deterministic content string based on the normalized candidate ingredient data, the normalized candidate nutritional data, and the normalized candidate preparation instruction data. Further, the obtaining of the at least one output may include computing a content-based meal fingerprint data by applying a deterministic hash function to the deterministic content string. Further, the obtaining of the at least one output may include retrieving, from the user meal history data within the predefined temporal lookback window, a plurality of historical content-based meal fingerprint data, loading the plurality of historical content-based meal fingerprint data into a hash-based lookup structure, and evaluating a membership of the content-based meal fingerprint data in the hash-based lookup structure to identify duplication. Further, when duplication is identified, the obtaining of the at least one output may include generating, based on at least one constraint, the replacement candidate meal data by modifying the at least one constraint to exclude an ingredient combination associated with the content-based meal fingerprint data for which duplication is identified, and inputting the at least one constraint as at least part of the at least one instruction to the at least one large language model. Further, the at least one of the candidate meal data and the replacement candidate meal data is configured to satisfy the tri-level validation against the cuisine signature data and, when duplication is identified, to avoid duplication within the predefined temporal lookback window as determined by the membership evaluation. Further, the at least one large language model is further configured for tailoring the at least one output based on the at least one characteristic by enforcing at least one output formatting constraint governed by the at least one parameter. Further, the method may include transmitting, using the communication device, the at least one output to the client device.

Further, in some embodiments, the method may include analyzing, using the processing device, the at least one input using at least one natural language processing model, wherein the at least one input is received from the client device and comprises at least one query corresponding to the meal planning request data specifying a geographic sub-regional cuisine identity and a multi-day planning duration. Further, the method may include determining, using the processing device, at least one action associated with the user based on the analyzing of the at least one input, wherein the at least one action comprises at least one of selecting the candidate meal data from the candidate meal data set, validating the candidate meal data against the cuisine signature data by executing the tri-level validation, generating the non-duplicate validated meal data, and rebalancing the nutritional target across remaining days of the multi-day planning duration. Further, the method may include determining, using the processing device, at least one action data associated with the at least one action based on the determining of the at least one action, wherein the at least one action data comprises at least one of a candidate ingredient data, a candidate nutritional data, a candidate preparation instruction data, a cuisine signature score, a content-based meal fingerprint data, a duplication indicator, an ingredient chain identifier, a remaining quantity value, and a nutritional deviation. Further, the determining of the at least one action data may be initiated based on at least one contextual variable, wherein the at least one contextual variable represents a condition relevant to the determining of the at least one action. Further, the at least one contextual variable may include a physical state of the client device, wherein the client device comprises at least one sensor for generating the physical state of the client device, and wherein the physical state is usable to control at least one of (i) receipt of the meal planning request data, (ii) transmission of the meal plan data, (iii) selection of the multi-day planning duration, and (iv) selection of the predefined temporal lookback window. Further, the physical state comprises at least one of an available network bandwidth state and a connectivity state indicating a communication capability of the client device for exchanging the user meal history data and the meal plan data, and further comprises at least one of a battery state and a processor utilization state indicating an execution capability of the client device for locally rendering the meal plan data and for locally storing the user meal history data. Further, the method may include analyzing, using the processing device, the at least one action data by forming a deterministic content string based on a normalized candidate ingredient data, a normalized candidate nutritional data, and a normalized candidate preparation instruction data and applying a deterministic hash function to the deterministic content string to compute the content-based meal fingerprint data, and by evaluating a membership of the content-based meal fingerprint data in a hash-based lookup structure storing a plurality of historical content-based meal fingerprint data within a predefined temporal lookback window to identify duplication. Further, the method may include determining, using the processing device, at least one status based on the analyzing of the at least one action data, wherein the at least one status comprises at least one of a tri-level validation pass status, a duplication status, a remaining quantity status associated with the ingredient chain identifier, and a nutritional deviation status relative to the nutritional target. Further, the method may include generating, using the processing device, the at least one output, wherein the at least one output comprises meal plan data comprising the plurality of non-duplicate validated meal data, and wherein generating the at least one output further comprises, when the duplication status indicates duplication, generating a replacement candidate meal data based on at least one constraint defined to cause the replacement candidate meal data to satisfy the tri-level validation and to avoid duplication within the predefined temporal lookback window.

Further, in some embodiments, the method may include analyzing, using the processing device, the at least one input received from the client device, wherein the at least one input comprises at least one of the meal planning request data and a request to access or update the user meal history data associated with the user. Further, the method may include determining, using the processing device, a requirement for authenticating the user based on the analyzing of the at least one input, wherein the requirement for authenticating the user is determined based on at least one of (i) a sensitivity associated with the user meal history data, (ii) a requested modification to the user meal history data, and (iii) an access context associated with the meal planning request data. Further, the method may include selecting, using the processing device, at least one authentication prompt from a plurality of authentication prompts for authenticating the user using at least one machine learning model based on the determining of the requirement. Further, the at least one machine learning model is configured for matching at least one requirement characteristic with at least one authentication prompt characteristic of each of the plurality of authentication prompts, wherein the at least one requirement characteristic comprises at least one of a requested operation type, a requested data scope, and a risk level, and wherein the at least one authentication prompt characteristic comprises at least one of a prompt modality, an interaction complexity, and an expected response type. Further, the plurality of authentication prompts may be disparate in at least one of the prompt modality and the expected response type. Further, the method may include transmitting, using the communication device, the at least one authentication prompt to the client device. Further, the method may include receiving, using the communication device, at least one response for the at least one authentication prompt from the client device. Further, the method may include analyzing, using the processing device, the at least one response. Further, the method may include generating, using the processing device, an authentication status of the user based on the analyzing of the at least one response. Further, the method may include executing, using the processing device, at least one operation based on the authentication status, wherein the at least one operation comprises at least one of (i) permitting receipt of the user meal history data from the client device, (ii) permitting storing of the plurality of non-duplicate validated meal data in association with the user in the user meal history data, and (iii) permitting transmitting of the meal plan data to the client device associated with the user.

Further, in some embodiments, the method may include obtaining, using the processing device, at least one authentication prompt characteristic of the at least one authentication prompt based on the selecting of the at least one authentication prompt. Further, the method may include analyzing, using the processing device, the at least one authentication prompt characteristic. Further, the method may include generating, using the processing device, at least one control data for controlling the at least one client device used for responding to the at least one authentication prompt based on the analyzing of the at least one authentication prompt characteristic. Further, the method may include embedding, using the processing device, the at least one control data in the at least one authentication prompt based on the generating of the at least one control data. Further, the method may include generating, using the processing device, at least one modified authentication prompt for the authenticating the at least one user through the at least one client device based on the embedding. Further, the at least one modified authentication prompt may include the at least one authentication prompt and the at least one control data embedded in the at least one authentication prompt. Further, the method may include transmitting, using the communication device, the at least one modified authentication prompt to the at least one client device. Further, the at least one client device is configured for analyzing the at least one modified authentication prompt. Further, the at least one client device is configured for extracting the at least one control data from the at least one modified authentication prompt based on the analyzing of the at least one modified authentication prompt. Further, the at least one client device may be configured for determining one or more temporary state parameters based on the at least one control data. Further, the at least one client device may be configured for storing a representation of a prior operational state of the at least one client device comprising one or more values of one or more device state parameters. Further, the at least one client device is configured for configuring the at least one client device to operate in a temporary operational state by updating at least one of the one or more device state parameters according to the one or more temporary state parameters. Further, at least one updated value of at least one of the one or more device state parameters controls an execution of a response generation operation for the at least one authentication prompt. Further, the at least one client device is configured for executing the response generation operation while the at least one client device operates in the temporary operational state for generating the at least one response corresponding to the at least one authentication prompt. Further, the at least one device is configured to automatically restore the prior operational state by restoring the one or more values of the one or more device state parameters after the generating of the at least one response. Further, in some embodiments, the at least one client device comprises the client device associated with the user, and the at least one authentication prompt corresponds to the at least one authentication prompt transmitted to the client device as described above, such that the generating of the at least one modified authentication prompt is performed as an alternative to, or as a refinement of, transmitting the at least one authentication prompt.

Further, in some embodiments, the method may include obtaining, using the processing device, at least one data comprising at least one of the candidate ingredient data, the candidate nutritional data, and the candidate preparation instruction data of the candidate meal data selected from the candidate meal data set. Further, the method may include obtaining, using the processing device, at least one additional data comprising at least one of the cuisine signature data and the weighted characteristic ingredient data defined by the cuisine signature data corresponding to the geographic sub-regional cuisine identity. Further, the method may include generating, using the processing device, a feature tensor for the at least one data, wherein the feature tensor is generated based on a canonical representation of the candidate ingredient data comprising canonical ingredient name data and canonical ingredient quantity data. Further, the method may include generating, using the processing device, an additional feature tensor from the at least one additional data, wherein the additional feature tensor is generated based on the weighted characteristic ingredient data and at least one cuisine-specific weight parameter defined in the cuisine signature data. Further, the feature tensor may include a plurality of feature vectors, and the additional feature tensor may include a plurality of additional feature vectors. Further, the method may include forming, using the processing device, a plurality of feature pairs by pairing a respective feature vector of the feature tensor with a respective additional feature vector of the additional feature tensor according to a mapping defining a correspondence between ingredients represented in the candidate ingredient data and characteristic ingredients represented in the cuisine signature data. Further, the method may include executing, using the processing device, at least one machine learning model based on the plurality of feature pairs based on the forming, wherein the at least one machine learning model is configured for computing a redundancy metric for each of the plurality of feature pairs by executing a similarity operation on a feature vector and an additional feature vector of each of the plurality of feature pairs. Further, the method may include comparing, using the processing device, the redundancy metric to a redundancy threshold defined by one or more learned parameters of the at least one machine learning model. Further, the method may include generating, using the processing device, a feature-selection mask for at least one feature pair of the plurality of feature pairs based on the redundancy metric and the redundancy threshold. Further, the feature-selection mask indicates, for each of the at least one feature pair having the redundancy metric satisfying the redundancy threshold, an exclusion of one feature vector from each of the at least one feature pair. Further, the method may include applying, using the processing device, the feature-selection mask to produce a reduced inference feature set by omitting, from further processing for calculating the cuisine signature score, feature vectors excluded by the feature-selection mask. Further, the method may include calculating, using the processing device, the cuisine signature score based on the reduced inference feature set as part of the tri-level validation by executing a reduced number of similarity operations and a reduced number of weighted aggregation operations relative to calculating the cuisine signature score without applying the feature-selection mask. Further, the method may include comparing, using the processing device, the cuisine signature score to the configurable authenticity threshold, and rejecting the candidate meal data when the tri-level validation fails and classifying the candidate meal data as an authentic candidate meal data when the tri-level validation passes.

Further, in some embodiments, the feature-selection mask may include at least one of a bitmask aligned to an ordering of the plurality of feature vectors and the plurality of additional feature vectors, and an index list identifying excluded feature vectors. Further, the applying of the feature-selection mask may include executing at least one gather operation to form the reduced inference feature set from non-excluded feature vectors of the plurality of feature vectors and the plurality of additional feature vectors.

Further, in some embodiments, the applying of the feature-selection mask reduces at least one of an inference latency, a memory bandwidth utilization, and a power consumption during the obtaining the at least one output using the at least one machine learning model, relative to the obtaining the at least one output without applying the feature-selection mask, by reducing a number of arithmetic operations and a memory accesses performed during the calculating of the cuisine signature score and/or the executing of the tri-level validation.

### Overview:

The present disclosure describes a comprehensive hybrid AI-powered and database-driven meal planning system that enforces cultural authenticity at sub-regional granularity, optimizes ingredient usage through forward-chaining leftover prediction, enables constraint-preserving meal substitutions, and prevents meal repetition using content-based fingerprinting with temporal deduplication.

Further, existing meal planning systems treat cuisines as monolithic categories (e.g., “Indian food”), ignoring critical sub-regional variations (Kerala vs. Tamil Nadu vs. Punjab). Further, current systems generate repetitive meal plans, causing user fatigue and abandonment. Further, meal substitution features break nutritional balance and ignore cultural constraints, Further, ingredient waste is significant due to lack of forward planning across multi-day meal cycles.

Further, the present disclosure describes the following known solutions and the drawbacks associated with them:
Generic cuisine tags: May not distinguish between Kerala coconut-based cooking vs. Punjab ghee-based cooking.
Name-based duplicate detection: Fails when same dish has different names or different dishes have similar names.
Simple meal swap features: Allow nutritional drift; user may unknowingly exceed calorie/protein targets.
Daily meal planning: Each day planned independently; no consideration of leftover ingredients; significant food waste.
AI-only meal generation: No cultural validation; AI generates plausible-sounding but inauthentic dishes.

Further, the need for the disclosed system arises due to:
The growing demand for personalized, culturally-authentic meal planning (especially for diaspora communities).
Health-conscious users require precise nutritional tracking that survives meal customization.
Sustainability concerns demand reduced food waste through intelligent ingredient management.
User retention requires variety without repetition across weeks of meal plans.

Further, in some embodiments, the disclosed system may include four integrated innovations:

### Innovation 1: Sub-Regional Cuisine Fingerprinting System

A tri-level validation framework that enforces cultural authenticity at geographic sub-regional granularity. Each sub-region (e.g., Kerala, Tamil Nadu, Punjab) is defined by an ingredient signature vector containing:
   Mandatory ingredients (MUST be present for authenticity).
   Prohibited ingredients (MUST NOT be present).
   Characteristic spices with weighted scores.
   Configurable authenticity threshold (0.65-0.90).

### Validation Process:

Step 1 (Hard): Check if meal contains any prohibited ingredients→REJECT if found
   Step 2 (Hard): Check if meal contains all mandatory ingredients→REJECT if missing
   Step 3 (Soft): Calculate signature score from characteristic ingredients→REJECT if below threshold

### Innovation 2: Forward-Chaining Leftover Optimization

A predictive algorithm that minimizes ingredient waste by projecting consumption across N-day planning periods:
   Aggregate total ingredient requirements across all N days.
   Calculate optimal batch sizes considering standard grocery packaging (500 g, 1 kg, 2 kg).
   Assign unique chain identifiers to track ingredient lineage.
   For each day, determine if leftovers satisfy requirements before purchasing new.
   When leftover consumption causes nutritional deviation, distribute corrections across future days.

### Innovation 3: Constraint-Preserving Bounded Meal Substitution

A multi-dimensional filtering system that enables user meal customization while preserving nutritional and cultural constraints:

### Bounded Constraints:

Per-meal: ±50 kilocalories, ±5 grams' protein from original meal.
   Per-week: ±1% of weekly nutritional target (prevents cumulative drift).

### Six-Filter Cascade:

Filter 1: Calorie bound check|Filter 2: Protein bound check|Filter 3: Cuisine type match.
   Filter 4: Dietary restriction check|Filter 5: Intra-plan deduplication|Filter 6: Weekly variance check
Intra-Plan Deduplication: Extracts all meal names from current 7-day plan to create exclusion list, preventing same meal from appearing twice in one week.

### Innovation 4: Historical Meal Deduplication Engine

A content-based fingerprinting system that prevents meal repetition across temporal planning sessions:

### Fingerprint Generation:

1. Normalize ingredients (lowercase, sorted alphabetically).
   2. Aggregate: meal_name+ingredients+calories+protein+instructions [0:100].
   3. Apply 32-bit hash function→8-character hexadecimal fingerprint Technical Specifications:
      Hash-based Set data structures for O (1) constant-time lookup.
      Configurable lookback window (default 14 days).
      Configurable retry limit (default 3 attempts).
      32-bit hash provides 4.29 billion possible fingerprints (extremely low collision probability).

Further, the present disclosure describes the following advantages of the disclosed system over known solutions:
Sub-Regional Cuisine Fingerprinting: First system to validate cuisine authenticity at the state/district level rather than the national level.
Forward-Chaining Leftover Optimization: Reduces ingredient waste by 30%+ compared to independent daily planning.
Constraint-Preserving Bounded Swaps: Guarantees nutritional balance survives user customization.
Historical Meal Deduplication: Content-based fingerprinting detects duplicates regardless of naming variations.

Further, in some embodiments, the present disclosure describes a system that facilitates:
Content-based meal fingerprinting using normalized ingredient vectors for duplicate detection.
Sub-regional cuisine validation with tri-level constraint checking (mandatory/prohibited/characteristic).
Bounded meal substitution with intra-plan deduplication and per-meal+per-week nutritional bounds.
Forward-chaining leftover optimization with unique chain identifiers for ingredient tracking.

In some embodiments, the present disclosure describes a system and method for culturally authentic meal planning with predictive resource optimization, sub-regional cuisine fingerprinting, and historical deduplication.

Further, the present disclosure describes a computer-implemented system and method for generating culturally-authentic meal plans with predictive resource optimization, comprising four primary innovations:
Sub-Regional Cuisine Fingerprinting: A method for enforcing cultural authenticity at multi-dimensional granularity by defining ingredient signature vectors for sub-regional cuisines, maintaining mandatory ingredient lists (must-include) and prohibited ingredient lists (must-exclude), and applying tri-level validation comprising prohibited ingredient check (hard constraint), mandatory ingredient checks (hard constraint), and signature score comparison (soft constraint). The system as currently implemented distinguishes between geographic sub-regions (Kerala versus Tamil Nadu versus Punjab). The disclosed system further contemplates extension to religious/cultural sub-groups within those regions (such as Palakkad Iyer Brahmin versus general Tamil cuisine) as future embodiments. In benchmark evaluations of the geographic validation system, observed classification accuracy exceeded 90% across sub-regional categories. No prior art enforces cuisine authenticity at the given granularity with the tri-level validation framework described herein.
Forward-Chaining Leftover Optimization: A predictive algorithm for minimizing ingredient waste in multi-day meal planning by aggregating total ingredient requirements across N days, calculating optimal initial batch sizes considering standard packaging quantities, assigning unique chain identifiers to track ingredient consumption lineage across days, determining when leftovers from previous days satisfy current requirements without new purchases, and dynamically rebalancing caloric and protein targets across future days when leftover consumption causes deviation from daily targets. Validated waste reduction observed in testing compared to independent daily planning.
Constraint-Preserving Bounded Meal Substitution: A meal substitution algorithm enabling user customization while preserving nutritional and cultural constraints by obtaining candidate swap meals from one or more sources (database queries and/or AI model generation), defining swap boundaries comprising per-meal deviation limits and weekly variance limits, extracting all meal names from the current plan to create an exclusion list (intra-plan deduplication), validating candidates against multi-dimensional constraints (calories, protein, cuisine match, dietary restrictions, intra-plan exclusion, weekly impact), and ranking valid candidates by similarity score. The algorithm enables safe meal customization without requiring manual nutritional recalculation or expert intervention.
Historical Meal Deduplication Engine: A content-based fingerprinting system for preventing duplicate meals across temporal meal planning sessions by generating deterministic 8-character hexadecimal fingerprints from normalized ingredient vectors, tracking user meal history with configurable lookback windows (default 14 days), implementing intelligent configurable retry logic (e.g., 3 attempts) when duplicates are detected. The system uses a 32-bit hash (2\hat{ }32≈4.29 billion possible values) providing extremely low practical collision probability for typical user meal histories while maintaining O(n) computational complexity and browser compatibility.

Further, the disclosed system relates to computational meal planning systems, and more particularly to systems and methods for generating culturally-authentic meal plans with automated leftover optimization, sub-regional cuisine validation, constraint-preserving meal substitution, and temporal deduplication across multi-day planning periods.

Further, the present disclosure describes the following problems with the existing solutions:

### Problem 1: Cultural Authenticity Enforcement

Existing meal planning systems treat cuisine as a simple categorical variable (‘Indian’, ‘Asian’, ‘Mexican’), failing to capture the nuanced nature of cultural food identity. Current systems may not distinguish between:
   Geographic sub-regions: Kerala cuisine vs Tamil Nadu cuisine vs Punjab cuisine (distinct states with unique ingredient profiles and cooking methods).
   Religious/cultural dietary requirements: Vegetarian, Jain, Brahmin, and other religious dietary constraints that intersect with geographic preferences
Leading to culturally inauthentic meal combinations that violate regional culinary traditions. Users seeking authentic regional cuisine receive generic approximations that fail to respect cultural boundaries.

### Problem 2: Ingredient Waste and Leftover Management

Existing meal planners generate daily meal plans without considering ingredient batch sizes or cross-day utilization, leading to:
   Unused ingredient portions (e.g., purchasing 500 g chicken when recipe needs 300 g, with no plan for remainder).
   No forward-planning for leftover utilization across subsequent days.
   Manual user intervention required to avoid waste.
   No mechanism to track ingredient lineage from purchase through consumption.
The given approach typically results in significant ingredient waste—often in the range of 25-40%—when each day is planned independently without cross-day ingredient optimization.

### Problem 3: Constraint Violations During Meal Swaps

When users request meal substitutions in existing systems, they face a trilemma:
   Unrestricted swaps: Allow any substitution, which violates carefully calculated dietary goals.
   Prohibited swaps: Completely prevent substitutions to maintain constraints, resulting in poor user experience.
   Manual recalculation: Require users to manually recalculate daily and weekly nutrition totals, requiring expert intervention.
No existing system provides bounded flexibility that preserves nutritional goals while enabling user customization within safe limits.

### Problem 4: Meal Repetition Across Planning Sessions

When AI systems generate meal plans for returning users, they lack persistent memory of previous recommendations, resulting in:
   Recipe repetition: Users receive the same meals (e.g., Dal Makhani variations) across multiple weeks.
   User fatigue: Perception that the AI is ‘not creative’ or ‘stuck in a loop’.
   ID-based deduplication failures: Meal names like ‘Palak Paneer’ vs ‘Palak Paneer Curry’ vs ‘Creamy Palak Paneer’ bypass naive deduplication.
   Stateless generation: Each AI invocation has no context of user's meal history.

Further, there is a need for a meal planning system that:
Enforces sub-regional cuisine authenticity using ingredient signature vectors with mandatory, prohibited, and characteristic ingredient validation.
Predictively optimizes ingredient batch sizes and leftover utilization across multiple days with trackable chain identifiers.
Enables constrained meal swaps that preserve both per-meal and weekly nutritional boundaries without expert intervention.
Maintains persistent memory of user meal history with content-based deduplication to eliminate repetition across temporal planning sessions.

In some embodiments, the present disclosure describes a comprehensive meal planning system comprising four primary innovations:

### Innovation 1: Sub-Regional Cuisine Fingerprinting

A method for enforcing cultural authenticity at sub-regional granularity by: Implemented embodiments (Geographic Sub-Regional Validation):
   Operating at geographic sub-regions (Kerala, Tamil Nadu, Punjab, Kashmir) with state-level or district-level granularity.
   Defining ingredient signature vectors for each geographic sub-region with mandatory, prohibited, and characteristic ingredient lists.
   Calculating cuisine authenticity scores with configurable thresholds (0.65-0.75 for broad regional, 0.80-0.85 for state-level).
   Applying tri-level validation: prohibited check (hard), mandatory check (hard), signature score (soft).
   Rejecting meals that fail authenticity thresholds and invoking the meal generation engine with updated constraints

### Contemplated Future Embodiments (Religious/Cultural Extension)

The framework is designed to be extensible to religious/cultural sub-groups (Brahmin vegetarian, Jain dietary rules) using the same tri-level validation algorithm.
   Contemplated embodiments would encode religious dietary constraints as computable rules with higher authenticity thresholds (0.90+).

### Innovation 2: Forward-Chaining Leftover Optimization

A predictive algorithm that:
   Aggregates total ingredient requirements across N-day planning periods.
   Calculates optimal initial batch sizes considering standard grocery packaging quantities (500 g, 1 kg, 2 kg).
   Assigns unique chain identifiers to track ingredient lineage from purchase through consumption.
   Projects forward to identify future consumption slots compatible with leftover ingredients.
   Determines when leftovers from previous days satisfy current requirements (avoiding new purchases).
   Dynamically rebalances caloric and protein targets across future days when leftover consumption causes deviation.
   Minimizes ingredient waste through forward-looking meal sequencing.

### Innovation 3: Constraint-Preserving Bounded Swaps

A meal substitution algorithm that:
   Obtains candidate swap meals from one or more sources: database queries and/or AI model generation.
   Defines per-meal swap boundaries (maximum calorie deviation, maximum protein deviation).
   Defines weekly variance limits to prevent cumulative drift from nutritional targets.
   Extracts all meal names from current plan to create exclusion list (intra-plan deduplication).
   Validates cuisine type matching to preserve cultural authenticity.
   Validates user-specific dietary restrictions (allergies, religious requirements).
   Calculates weekly nutritional impact before allowing substitution.
   Ranks valid candidates by similarity score to original meal
   Enables user customization without expert intervention while preserving goals

### Innovation 4: Historical Meal Deduplication Engine

A content-based fingerprinting system that:
   Generates deterministic 8-character hexadecimal fingerprints from normalized ingredient vectors, nutritional content, and preparation methods.
   Tracks user meal history with configurable temporal lookback windows (default 14 days).
   Implements intelligent retry logic with configurable attempts (e.g., 3 attempts) and configurable delay when duplicates detected.
   Uses hash-based Set data structures for O (1) constant-time fingerprint lookup complexity.
   Records successfully generated meals with timestamps and nutritional metadata for future deduplication.
   Provides extremely low practical collision probability with 32-bit hash (2\hat{ }32≈4.29 billion possible fingerprints) for typical user meal histories.
   Integrates seamlessly with cuisine validation, leftover optimization, and meal swap systems.

In some embodiments, the disclosed system is implemented as a software system executing on one or more processors within a computing environment. The system comprises a meal generation engine (which may utilize artificial intelligence models, database queries, or hybrid approaches) and validation modules that enforce nutritional, cultural, and deduplication constraints. Data structures including hash-based Sets enable constant-time O (1) lookup operations for efficient fingerprint comparison. The following sections describe each innovation in detail:

### 1. Sub-Regional Cuisine Fingerprinting System

a. Ingredient Signature Vectors
Each sub-regional cuisine is defined by an ingredient signature vector comprising:
   Mandatory ingredients: Ingredients that MUST be present for cultural authenticity.
   Prohibited ingredients: Ingredients that MUST NOT be present (cultural or religious violation).
   Characteristic spices: Weighted ingredients that contribute to authenticity score.
   Authenticity threshold: Minimum signature score required for acceptance (0.65-0.90).

### Implemented Embodiments (Geographic Sub-Regional Validation)

The system as currently reduced to practice enforces geographic sub-regional cuisine authenticity using the tri-level validation framework. The following examples illustrate implemented geographic signatures:

### Example: Kerala Cuisine Signature (Implemented)

region: ‘Kerala’
   dimension: ‘geographic’
   mandatory_ingredients: [curry_leaves, coconut_oil, kokum,
   black_mustard_seeds]
   prohibited_ingredients: [ghee, paneer, kashmiri_chili]
   characteristic_spices: [{black_pepper: 0.9}, {cardamom: 0.8}, {cinnamon: 0.7}]
   authenticity_threshold: 0.85

### Example: Tamil Nadu Cuisine Signature (Implemented)

region: ‘Tamil Nadu’
   dimension: ‘geographic’
   mandatory_ingredients: [curry_leaves, tamarind, sambar_powder, urad_dal]
   prohibited_ingredients: [coconut_oil]/Uses sesame oil instead
   characteristic_spices: [{fenugreek: 0.9}, {asafoetida: 0.8}, {black_pepper: 0.7}]
   authenticity_threshold: 0.85

### Example: Punjab Cuisine Signature (Implemented)

region: ‘Punjab’
   dimension: ‘geographic’
   mandatory_ingredients: [ghee, garam_masala, cumin, coriander]
   prohibited_ingredients: [coconut_oil, kokum, sambar_powder]
   characteristic_spices: [{kashmiri_chili: 0.9}, {amchur: 0.8}, {kasoori_methi: 0.7}]
   authenticity_threshold: 0.85

### Contemplated Future Embodiments (Religious/Cultural Sub-Group Validation)

The disclosed system further contemplates extension of the signature vector system to encode religious and cultural dietary constraints. In contemplated embodiments, the system would validate meals against religious sub-group requirements as illustrated in the following examples:
   Contemplated Example: Palakkad Iyer Cuisine Signature (Brahmin Vegetarian):
   region: ‘Palakkad Iyer’
   dimension: ‘religious_cultural’/CONTEMPLATED
   sub_culture: ‘Tamil Brahmin’
   mandatory_ingredients: [curry_leaves, ghee, tamarind, rice, urad_dal]
   prohibited_ingredients: [onion, garlic, meat, eggs]/Religious restriction
   religious_constraints: ‘brahmin_vegetarian’
   authenticity_threshold: 0.90/Higher for religious specificity

### Contemplated Example: Jain Cuisine Signature

region: ‘Jain’
   dimension: ‘religious_cultural’/CONTEMPLATED
   mandatory_ingredients: [ghee, cumin, coriander]
   prohibited_ingredients: [onion, garlic, potato, carrot, beetroot]/Root vegetables
   religious_constraints: ‘jain_vegetarian’
   authenticity_threshold: 0.90

### Multi-Dimensional Granularity:

The ingredient signature vectors are designed to support multiple dimensions of cultural identity:
   Geographic dimension (IMPLEMENTED): Sub-regional cuisines at state or district level (Kerala, Tamil Nadu, Punjab, Kashmir).
   Religious/cultural dimension (CONTEMPLATED): Sub-communities within geographic regions defined by religious dietary laws (Brahmin vegetarian, Jain, specific caste-based food traditions).
   Intersection of dimensions (CONTEMPLATED): Cuisines defined by BOTH geography AND religion (e.g., Palakkad Iyer=Tamil region+Brahmin vegetarian constraints).
The tri-level validation framework is designed to accommodate both implemented geographic validation and contemplated religious/cultural extensions using the same algorithmic approach.
   b. Tri-Level Validation Algorithm
The validation algorithm as implemented operates on geographic sub-regions (Kerala, Tamil Nadu, Punjab). The same algorithmic framework is designed to accommodate contemplated extensions to religious/cultural sub-groups (Palakkad Iyer Brahmin, Jain, etc.) without modification to the core validation logic.
The algorithm applies three levels of constraints in sequence:
   Level 1—Prohibited Ingredient Check (Hard Constraint): The system scans the meal's ingredient list for any ingredient appearing in the prohibited list. In implemented embodiments, prohibited ingredients are defined by geographic regional rules (e.g., kashmiri_chili prohibited in Kerala cuisine, coconut_oil prohibited in Tamil Nadu cuisine). In contemplated embodiments, the same check would apply to religious dietary laws (e.g., onion and garlic prohibited in Brahmin vegetarian cuisine).
   If ANY prohibited ingredient is found, the meal is immediately rejected and flagged for regeneration. Prohibited ingredient check is a hard constraint with zero tolerance.
   Level 2—Mandatory Ingredient Check (Hard Constraint): The system verifies that ALL mandatory ingredients are present in the meal. In implemented embodiments, mandatory ingredients define the geographic signature (e.g., curry_leaves required for South Indian cuisines). In contemplated embodiments, the same check would validate religious/cultural mandatory ingredients (e.g., ghee required for certain Brahmin preparations). If any mandatory ingredient is missing, the meal is rejected.
   Level 3—Signature Score Calculation (Soft Constraint): For meals passing both hard constraints, the system calculates a signature score by: (a) extracting the meal's ingredient vector, (b) comparing against the cuisine's characteristic spice vector (weighted by cultural importance), (c) computing weighted sum of matching characteristic ingredients, (d) comparing the resulting score against the authenticity threshold.
      c. Novel Aspects
The sub-regional cuisine fingerprinting system is novel because:
   Geographic sub-regional granularity (IMPLEMENTED): System validates at state-level granularity (Kerala vs Tamil Nadu vs Punjab) rather than broad national categories.
   Tri-level validation framework: Combination of two hard constraints plus one soft constraint with configurable thresholds—applicable to both implemented geographic and contemplated religious validation.
   Extensible architecture (CONTEMPLATED): Framework designed to accommodate religious constraint encoding (Brahmin, Jain, and other religious dietary rules) as computable constraints.
   Quantifiable authenticity: Provides numerical authenticity score (0.0 to 1.0) with configurable thresholds based on specificity level.
      d. Design Principles
The sub-regional cuisine fingerprinting system addresses a significant gap in how computational systems handle cultural identity. Prior meal planning systems treat cuisine as a simple categorical variable (‘Indian’, ‘Mexican’, ‘Italian’), ignoring the regional distinctions that define authentic food experiences.

### Key Design Principles:

Sub-regional granularity (IMPLEMENTED): Users specify sub-regional identity (Kerala, Tamil Nadu, Punjab) rather than broad national categories
   Extensible taxonomy (CONTEMPLATED): Architecture supports extension to religious/cultural dimensions (Brahmin vegetarian, Jain dietary rules)
   Computable constraints: Mandatory and prohibited ingredient lists encode cultural requirements as machine-verifiable rules
   Authenticity scoring: Quantifiable signature scores provide transparency into cuisine classification decisions
The given approach treats cultural identity as a first-class system component rather than an afterthought.

### 2. Forward-Chaining Leftover Optimization Engine

a. Problem Statement
Traditional meal planners treat each day independently, resulting in significant ingredient waste:
   Day 1: Cook chicken curry (needs 300 g)->Buy 500 g->200 g wasted or manually saved.
   Day 2: Cook chicken stir-fry (needs 250 g)->Buy new 500 g->Leftover from Day 1 ignored.
   Day 3: Cook chicken salad (needs 150 g)->Buy new 500 g->Cumulative waste grows.
   Result: 1500 g purchased, 700 g consumed, 800 g wasted (53% waste rate).
      b. Solution Architecture
The forward-chaining algorithm operates in four stages:
   Stage 1—Aggregate Requirements: Sum total ingredient needs across all N days of the planning period.
   Stage 2—Optimal Batch Sizing: Calculate purchase quantities considering standard packaging (500 g, 1 kg, 2 kg) to minimize excess while ensuring sufficiency.
   Stage 3—Chain Identifier Assignment: Assign unique tracking IDs (e.g., CHK-001 for chicken batch) to trace ingredient lineage from purchase through consumption across multiple meals.
   Stage 4—Forward Projection: For each day, determine if previous-day leftovers satisfy current requirements. If yes, consume from leftover (no new purchase). If no, calculate additional batch needed.
      c. Caloric Rebalancing Algorithm
When leftover consumption causes deviation from daily targets, the system distributes corrections across remaining days:

$$deviation = actual\_calories - target\_calories$$

$$correction\_per \_day = - deviation / remaining\_days$$

for each remaining_day:

$$adjusted\_target = original\_target + correction\_per \_day$$

Ensuring weekly totals are preserved even when individual days deviate due to ingredient batch constraints.
   d. Novel Aspect
The forward-chaining leftover optimization is novel because:
   Predictive batch sizing: No prior art considers standard packaging quantities when planning multi-day meals.
   Chain identifier tracking: First system to assign unique IDs for tracing ingredient consumption lineage.
   Caloric rebalancing: Automatic redistribution of nutritional targets when leftover consumption causes deviation.
   Forward-looking optimization: Plans future days based on projected leftovers rather than reactive waste management.

### 3. Historical Meal Deduplication Engine

a. Problem Statement
When AI systems generate meal plans for returning users, they lack persistent memory of previous recommendations. Resulting in:
   Recipe Repetition: Users receive the same meals (e.g., ‘Dal Makhani’ variations) across multiple weeks.
   User Fatigue: Perception that the AI is ‘not creative’ or ‘stuck in a loop’.
   ID-Based Deduplication Failures: Meal names like ‘Palak Paneer’ vs ‘Palak Paneer Curry’ vs ‘Creamy Palak Paneer’ bypass naive deduplication.
   Stateless Generation: Each AI invocation has no context of user's meal history.
      b. Solution Architecture: Five-Stage Pipeline

### STAGE 1: Pre-Generation History Retrieval

Query user's meal history for last N days (default 14 days).
   Extract meal fingerprints into hash-based Set data structure for O (1) constant-time lookup.
In preferred embodiments, the system first queries a proprietary recipe database for unique meals before invoking AI generation, ensuring cultural authenticity and variety from the start.

### STAGE 2: AI Meal Plan Generation

Invoke AI model with user constraints and preferences.
   Receive candidate meal plan.
   Apply existing cuisine validation (Innovation 1) and leftover optimization (Innovation 2).

### STAGE 3: Fingerprint Calculation

For each meal in candidate plan:
   Normalize Ingredients: Convert to lowercase, trim whitespace, standardize measurement units, sort alphabetically for determinism.
   Aggregate Content: Combine meal.name+normalized ingredients+calories+protein+instructions [0:100] into deterministic string.
   Generate Hash: Apply bitwise hash function to produce 8-character hexadecimal fingerprint (32-bit, 2\hat{ }32 possible values).

### STAGE 4: Duplicate Detection

Compare each candidate meal fingerprint against historical fingerprint Set.
   Flag duplicates with meal name.
   Count total duplicates found.

### STAGE 5: Retry or Commit

```
IF duplicates found:
  IF attempt < maxRetries:
    Invoke meal generation engine with updated constraints
  ELSE:
    THROW error (all retries exhausted)
IF no duplicates:
  RECORD meals in history with fingerprints
  RETURN unique meal plan to user
```

c. Fingerprint Generation Algorithm
The core innovation is the content-based fingerprint that captures meal essence:

```
function generateMealFingerprint(meal) {
// Step 1: Normalize all ingredients
const normalizedIngredients = meal.ingredients
  .map(ing => {
    const name = ing.name.toLowerCase( ).trim( );
    let amount = ing.amount.toLowerCase( ).trim( );
    // Unit standardization
    amount = amount
      .replace(/cups?/, ′cup′)
      .replace(/tablespoons?|tbsp/, ′tbsp′)
      .replace(/grams?|gm/, ′g′);
    return ′${name}:${amount}′;
  })
  .sort( ) // Alphabetical ordering for determinism
  .join(′|′);
// Step 2: Aggregate meal content
const contentString = [
  meal.name.toLowerCase( ).trim( ),
  normalizedIngredients,
  Math.round(meal.calories ∥ 0),
  Math.round(meal.protein ∥ 0),
  meal.instructions.toLowerCase( ).substring(0, 100).trim( )
].join(′::′);
// Step 3: Generate hash (browser-compatible)
let hash = 0;
for (let i = 0; i < contentString.length; i++) {
  const char = contentString.charCodeAt(i);
  hash = ((hash << 5) - hash) + char;
  hash = hash & hash; // 32-bit conversion
}
// Step 4: Return 8-character hex fingerprint
return Math.abs(hash).toString(16).padStart(8, ′0′);
}
```

### Key Properties:

Deterministic: Same meal content always produces same fingerprint.
   Low collision probability: 32-bit hash (2\hat{ }32≈4.29 billion possible fingerprints) sufficient for typical user meal histories.
   Fast: O(n) time complexity where n=total ingredient string length.
   Browser-Compatible: No Node.js crypto dependency.
      d. Integration with Other Innovations
Synergy with Innovation 1 (Sub-Regional Cuisine Fingerprinting):
   Deduplication operates after cuisine validation.
   Fingerprints respect sub-regional constraints (e.g., ‘Kerala Fish Curry’ vs ‘Malabar Fish Curry’ treated as distinct).
      Synergy with Innovation 2 (Forward-Chaining Leftover Optimization):
Deduplication tracks original meals, not leftover variants.
   Chain IDs preserved in meal history.
      Synergy with Innovation 3 (Constraint-Preserving Bounded Swaps):
When user swaps a meal, new fingerprint recorded immediately.
   Prevents swapped meal from reappearing in future weeks.
In certain embodiments, to further reduce the likelihood of duplicates and minimize computational overhead, the names or descriptions of recently served meals may optionally be included in the prompt provided to the artificial intelligence model, with the deterministic fingerprint comparison and retry mechanism serving as the primary safeguard against duplication.
   e. Novel Aspects
The historical meal deduplication system is novel because:
   Content-based fingerprinting: First system to use normalized ingredient vectors instead of meal names/IDs.
   Temporal tracking: Configurable lookback windows (14-60 days) with automatic expiration.
   Intelligent retry logic: Configurable max attempts balances variety with performance.
   Browser-compatible: No server-side cryptographic dependencies required.
   Low collision probability: 32-bit hash (2\hat{ }32≈4.29 billion possible fingerprints) sufficient for typical meal histories.
   Seamless integration: Works with cuisine validation, leftover optimization, and meal swap systems.

### 4. Constraint-Preserving Bounded Meal Substitution

a. Problem Statement
When users want to swap a meal in the plan, existing systems offer only extreme options:
   Unrestricted swaps: Any meal may replace any meal, destroying carefully calculated nutritional balance.
   Prohibited swaps: No swaps allowed, frustrating users who want flexibility.
   Manual recalculation: Users must manually track nutritional impact, requiring expert knowledge.
      b. Bounded Swap Architecture
The algorithm defines two levels of constraints:

### Per-Meal Bounds:

Maximum calorie deviation: ±50 kcal from original meal.
   Maximum protein deviation: ±5 g from original meal.

### Weekly Bounds:

Maximum weekly variance: ±1% of weekly target.
   Prevents cumulative drift from multiple per-meal deviations.
      c. Multi-Dimensional Filtering Cascade
Candidate swap meals may be obtained by querying a pre-existing meal database, by generating new meals via an AI model, or by a combination of both approaches. Regardless of source, all candidates pass through sequential filters:
   Filter 1: Calorie bound check (reject if |candidate−original|>50 kcal).
   Filter 2: Protein bound check (reject if |candidate−original|>5 g).
   Filter 3: Cuisine match check (reject if cuisine type differs).
   Filter 4: Dietary restriction check (reject if violates user restrictions).
   Filter 5: Intra-plan deduplication check (reject if meal name matches any meal in current plan).
   Filter 6: Weekly variance check (reject if swap would exceed ±1% weekly target).
      i. Intra-Plan Meal Deduplication
To prevent repetitive meals within the active planning cycle, the bounded meal swap system extracts all meal names from the current meal plan before querying swap candidates. The system iterates through all days in the plan (e.g., days 1-7 for a weekly plan) and collects meal names from all meal types (breakfast, lunch, dinner, snacks), creating an exclusion list.

### Algorithm:

1. Initialize empty exclusion list: mealsToAvoid=[ ]
   2. For each day d in planning cycle (day 1 to day N):
      a) Extract breakfast meal name->append to mealsToAvoid
      b) Extract lunch meal name->append to mealsToAvoid
      c) Extract dinner meal name->append to mealsToAvoid
      d) If snacks enabled, extract snack name->append to mealsToAvoid
   3. Remove duplicate entries from mealsToAvoid (create unique set)
   4. Store exclusion list for candidate filtering.

### Example: If the Current 7-Day Plan Contains

Breakfasts: Poha, Idli, Upma, Dosa, Paratha, Poha, Idli
   Lunches: Dal Bafla, Sambar Rice, Chole, Rajma, Paneer Curry, Dal Bafla, Sambar Rice
   Dinners: Mixed Dal, Vegetable Biryani, Palak Paneer, Dal Tadka, Jeera Rice, Mixed Dal, Biryani
The exclusion list becomes:

$$mealsToAvoid = [ ‘ Poha ’ , ‘ Idli ’ , ‘ Upma ’ , ‘ Dosa ’ , ‘ Paratha ’ , ‘ Dal Bafla ’ , ‘ Sambar Rice ’ , ‘ Chloe ’ , ‘ Rajma ’ , ‘ Paneer Curry ’ , ‘ Mixed Dal ’ , ‘ Vegetable Biryani ’ , ‘ Palak Paneer ’ , ‘ Dal Tadka ’ , ‘ Jeera Rice ’ , ‘ Biryani ’ ]$$

The given exclusion list is applied as an additional constraint during candidate filtering, ensuring swap suggestions do NOT match any existing meal in the current plan. When querying the meal database:
   WHERE name NOT IN (mealsToAvoid)
   Or for AI generation:
   CRITICAL RULE: The replacement meal MUST NOT match any of the existing meals:
   [list of mealsToAvoid]
Maintaining variety across the full 7-day planning window and prevents user fatigue from seeing the same meals multiple times within a single week.
Distinction from Inter-Plan Deduplication: The given intra-plan deduplication (name-based, within current week) operates independently from the inter-plan deduplication (Innovation 4, fingerprint-based, across different weeks). Together, the said systems ensure both short-term variety (within a week) and long-term variety (across multiple weeks).
   ii. Weekly Variance Calculation
To prevent nutritional drift across multiple swaps, the system calculates weekly variance impact:

$$weeklyVariance = \vert newWeeklyTotal - targetWeeklyTotal \vert /$$

targetWeeklyTotal
Candidates are filtered to ensure:

$$weeklyVariance ( calories ) <= 1 \% AND weeklyVariance ( protein ) <= 1 \%$$

### Example: For a 2100-Calorie Daily Target (14,700 Calories/Week)

Maximum allowed deviation: +/−147 calories
   Swap causing 200-calorie increase: REJECTED (1.36%>1%)
   Swap causing 100-calorie decrease: ACCEPTED (0.68%<1%)
      d. Similarity Scoring
Valid candidates are ranked by similarity to original meal:

$$similarity = 1 - ( ( calorie\_diff / 50 ) * 0.6 + ( protein\_diff / 5 ) * 0.4 )$$

Calories are weighted more heavily (60%) than protein (40%) based on typical user priorities.
   e. Novel Aspects
The constraint-preserving bounded swap system is novel because:
   Flexible candidate sourcing: Candidates obtained from database queries, AI model generation, or hybrid combination.
   Dual-level bounds: Both per-meal and weekly constraints prevent local and cumulative drift.
   Multi-dimensional filtering: Calories, protein, cuisine, dietary restrictions, intra-plan exclusion, and weekly impact are all enforced.
   Intra-plan deduplication: Prevents repetitive meals within the planning window by extracting all meal names from the current plan and using them as an exclusion filter, ensuring variety across the full week.
   Similarity ranking: Valid options presented in order of perceptual closeness to the original.
   No expert intervention: Users customize meals without a nutritionist's involvement.
      f. Design Principles
The bounded swap system addresses a limitation in traditional meal planning systems that force a binary choice: either users have full control (risking goal violation) or the system has full control (limiting user agency). The disclosed system implements a ‘bounded autonomy’ approach a middle ground where users retain meaningful choice within defined safety boundaries.
Key design principles implemented in the algorithm:
   Bounded flexibility: Deviation thresholds (+50 kcal, +5 g protein) selected to balance user flexibility with nutritional goal preservation.
   Transparent constraint communication: Users are informed WHY certain swaps are unavailable through explicit boundary messaging.
   Similarity-ranked presentation: Options presented in order of closeness to user's original selection.
   Graceful degradation: When constraints severely limit options, the system explains the limiting factor rather than failing silently.
   Cultural consistency: Cuisine matching ensures swaps honor the user's specified cultural identity.
      g. Integration with Other Innovations
Innovation 1 (Sub-Regional Fingerprinting): Swap candidates are filtered by geographic cuisine granularity. Kerala swaps maintain coconut-based dishes; Tamil Nadu swaps maintain tamarind-based dishes.
Innovation 2 (Forward-Chaining Leftovers): Swap candidates respect existing meal chains. Swapping a leftover source meal triggers recalculation of dependent meal chains.
Innovation 4 (Historical Deduplication): Swap candidates are filtered against user's meal history (14-day lookback), preventing suggesting meals recently served in previous weeks. Swapped meals are recorded in history with fingerprints.
Distinction between Intra-Plan and Inter-Plan Deduplication: Intra-plan deduplication (name-based, within current week) operates independently from inter-plan deduplication (Innovation 4, fingerprint-based, across different weeks). Together, the said systems ensure both short-term variety (within a week) and long-term variety (across multiple weeks).
   h. Implementation Status

### Core System (Reduced to Practice):

Bounded nutritional constraints (±50 cal, ±5 g protein) in swap candidate filtering.
   Cuisine preference enforcement in swap generation.
   Dietary restriction compliance (vegetarian, allergen exclusions).
   Basic duplicate avoidance (excluding the specific meal being swapped).
   Intra-plan meal name deduplication (meal name extraction from active plan to create exclusion list).
   Weekly nutritional variance calculation for swap candidates.

### Extended Integration (Contemplated Within Provisional Period):

Inter-plan historical deduplication integration (querying user's meal history fingerprints before generating swap candidates) —MealHistoryService infrastructure exists; integration with swap workflow pending.

In some embodiments, the present disclosure describes the following working examples:

### Example 1A: Geographic Sub-Regional Validation (IMPLEMENTED)

Test Case: Validate ‘Sambar’ meal for both Kerala and Tamil Nadu cuisines.
Meal Data: Name: ‘Sambar’, Ingredients: [toor_dal: 100 g, tamarind: 20 g, curry_leaves: 5 g, sambar_powder: 15 g, vegetables_mixed: 150 g, coconut_oil: 10 g], Calories: 180, Protein: 8 g

### Kerala Validation:

Step 1 (Prohibited): Check for [ghee, paneer, kashmiri_chili]->NONE FOUND->PASS
   Step 2 (Mandatory): Check for [curry_leaves, coconut_oil, kokum, black mustard_seeds]
      curry_leaves: PRESENT
      coconut_oil: PRESENT
      kokum: MISSING->FAIL
   Result: REJECTED for Kerala (missing kokum)

### Tamil Nadu Validation:

Step 1 (Prohibited): Check for [coconut_oil]->coconut_oil FOUND->FAIL
   Result: REJECTED for Tamil Nadu (contains prohibited coconut_oil) Conclusion: Same ‘Sambar’ recipe may be authentic for one geographic sub-region but not another, demonstrating the importance of sub-regional validation. The given example uses the currently implemented geographic validation.

### Example 1B: Religious/Cultural Validation

The following example illustrates how the tri-level validation framework could be extended to religious/cultural sub-groups in contemplated future embodiments:
Test Case: Validate ‘Vegetable Biryani’ meal for Palakkad Iyer (Tamil Brahmin) cuisine.
Meal Data: Name: ‘Vegetable Biryani’, Ingredients: [basmati rice: 200 g, mixed vegetables: 150 g, ghee: 20 g, onion: 50 g, garlic: 10 g, biryani_masala: 15 g],
Calories: 450, Protein: 12 g

### Contemplated Palakkad Iyer Validation:

Step 1 (Prohibited): Check for [onion, garlic, meat, eggs]
   onion: FOUND->FAIL
      garlic: FOUND->FAIL
   Result: REJECTED for Palakkad Iyer (contains prohibited onion and garlic)
The given example demonstrates how the same tri-level validation algorithm could accommodate religious dietary constraints in future implementations. The algorithmic framework remains unchanged; only the signature data differs.

### Example 2: Forward-Chaining Leftover Optimization

Scenario: 7-day meal plan with chicken appearing in Days 1, 3, and 5.
   Day 1: Chicken Curry needs 300 g
   Day 3: Chicken Stir-fry needs 250 g
   Day 5: Chicken Salad needs 200 g
   Total: 750 g
   Traditional approach: Buy 500 g×3=1500 g, waste 750 g (50%)
   Forward-chaining: Buy 1 kg (standard package), use all, waste 250 g (25%)
   Chain tracking:
      CHK-001: initial=1000 g
      Day 1: consume 300 g, remaining=700 g
      Day 3: consume 250 g from leftover, remaining=450 g
      Day 5: consume 200 g from leftover, remaining=250 g
      Final waste: 250 g (25% vs 50% traditional)

### Example 3: Historical Meal Deduplication (Multi-Week Scenario)

Scenario: A user receives weekly meal plans. They have already received 2 meal plans over the past 14 days. System is configured with maximum retry threshold of 3 attempts. User's meal history (last 14 days):

### Week 1:

Breakfast: Moong Dal Cheela (280 kcal, 14 g) —fingerprint: a1b2c3d4
   Lunch: Chana Masala with Roti (420 kcal, 16 g) —fingerprint: e5f6g7 h8
   Dinner: Tandoori Chicken (350 kcal, 38 g) —fingerprint: i9j0k1l2

### Week 2:

Breakfast: Besan Cheela (260 kcal, 12 g) —fingerprint: m3n4o5p6
   Lunch: Rajma with Brown Rice (380 kcal, 14 g) —fingerprint: q7r8s9t0
   Dinner: Palak Paneer (340 kcal, 18 g) —fingerprint: u1v2w3x4

$$recentFingerprints = \{ a 1 b 2 c 3 d 4 , e 5 f 6 g 7 h 8 , i 9 j 0 k 1 l 2 , m 3 n 4 o 5 p 6 , q 7 r 8 s 9 t 0 , u 1 v 2 w 3 x 4 \}$$

### Attempt 1: AI Generates Week 3 Plan

Breakfast: Moong Dal Cheela->fingerprint: a12c3d4->DUPLICATE!
   Lunch: Paneer Tikka->fingerprint: y5z6a7b8->Unique
   Dinner: Dal Tadka->fingerprint: c9d0e1f2->Unique
   Result: 1 duplicate found, RETRY
      Attempt 2: AI Regenerates with Different Seed
Breakfast: Stuffed Paratha->fingerprint: g3h4i5j6->Unique Lunch: Dal Makhani with Roti->fingerprint: k718m9n0->Unique Dinner: Palak Paneer->fingerprint: u1v2w3x4->DUPLICATE! Result: 1 duplicate found, RETRY (attempt 2/3)

### Attempt 3: AI Regenerates Again

Breakfast: Stuffed Paratha with Yogurt->fingerprint: o1p2q3r4->Unique
   Lunch: Dal Makhani with Roti->fingerprint: s5t6u7v8->Unique
   Dinner: Chicken Tikka (320 kcal, 35 g)->fingerprint: w9x0y1z2->Unique
   Result: 0 duplicates, SUCCESS!
   Record new fingerprints: {o1p2q3r4, s5t6u7v8, w9x0y1z2}
   Return unique meal plan to user

### Example 4: Constraint-Preserving Bounded Swap

Scenario: User wants to swap Day 3 Dinner in a Punjab cuisine plan.
Current Meal: ‘Grilled Chicken’ (400 kcal, 40 g protein, Punjab)
Weekly context: 13,850 kcal consumed so far, target 14,000 kcal
   Candidate 1: ‘Butter Chicken’ (650 kcal, 35 g protein)
      Calorie diff: |650-400|=250 kcal>50 kcal bound
      VERDICT: REJECTED (exceeds calorie bound)
   Candidate 2: ‘Tandoori Chicken’ (420 kcal, 38 g protein)

$$Calorie diff : \vert 420 - 400 \vert = 20 kcal < 50 kcal bound -> PASS$$

Protein diff: |38-40|=2 g<5 g bound->PASS
   Cuisine: Punjab->PASS
   Weekly impact: 13,850+420=14,270 kcal
      Variance: |14,270-14,000|/14,000=1.9%>1% bound
   VERDICT: REJECTED (exceeds weekly variance)
Candidate 3: ‘Chicken Tikka’ (390 kcal, 42 g protein)
   Calorie diff: |390-400|=10 kcal<50 kcal bound->PASS
   Protein diff: |42-40|=2 g<5 g bound->PASS
   Cuisine: Punjab->PASS
   Weekly impact: 13,850+390=14,240 kcal
      Variance: |14,240-14,000|/14,000=1.7%>1% bound
   VERDICT: REJECTED (exceeds weekly variance)
Candidate 4: ‘Reshmi Kebab’ (380 kcal, 41 g protein)
   Calorie diff: |380-400|=20 kcal<50 kcal bound->PASS
   Protein diff: |41-40|=1 g<5 g bound->PASS
   Cuisine: Punjab->PASS
   Weekly impact: 13,850+380=14,230 kcal
      Variance: |14,230-14,000|/14,000=1.6%>1% bound
   VERDICT: REJECTED (exceeds weekly variance)
In the given case, user has consumed too many calories earlier in the week, limiting swap options. System informs user that only lower-calorie options are available to stay within weekly goals.

In some embodiments, the present disclosure provides the following technical validation data:

### Validation 1: Geographic Sub-Regional Cuisine Classification Accuracy

### (Validation Performed on Implemented Geographic Sub-Regional Cuisines)

Test set: 500 meals across 5 geographic sub-regions.
Kerala meals: Observed accuracy exceeding 90%.
Tamil Nadu meals: Observed accuracy exceeding 85%.
Punjab meals: Observed accuracy exceeding 85%.
Kashmir meals: Observed accuracy exceeding 80%.
Gujarat meals: Observed accuracy exceeding 85%.
Overall: Observed accuracy exceeding 87% across geographic sub-regions.

### Validation 2: Forward-Chaining Waste Reduction

Test scenarios: 100 7-day meal plans.
Baseline (independent daily): Typical waste rate 25-40%.
With forward-chaining: Typical waste rate 10-20%.
Observed improvement: Significant waste reduction through batch optimization.

### Validation 3: Bounded Swap Constraint Preservation

Test swaps: 1000 user swap requests.
Per-meal bounds satisfied: >99%
Weekly bounds satisfied: >95%
Cuisine match maintained: 100%
Dietary restrictions respected: 100%

### Validation 4: Historical Meal Deduplication Effectiveness

Fingerprint Collision Rate (tested on 10,000 unique Indian meals):
   Total meals: 10,000
   Unique fingerprints: 9,998
   Collisions: 2 (0.02%)
Retry Distribution (tested with max 3 attempts):
   Success on attempt 1: ˜ 76%
   Success on attempt 2: ˜ 21%
   Success on attempt 3: ˜ 3%
   Failure (all retries exhausted): <0.1%

### Query Performance:

getUserRecentMeals (14 days): avg 12 ms (indexed query)
   generateMealFingerprint( ): avg 0.8 ms per meal
   recordMealPlanServed( ): avg 45 ms (batch insert)
   Total overhead per generation: ˜60 ms

In some embodiments, the present disclosure describes a computer-implemented method for enforcing cultural authenticity in meal planning at sub-regional granularity. Further, the method may include defining a plurality of cuisine signatures for geographic sub-regions. Further, each signature may include:
A list of mandatory ingredients characteristic of the geographic sub-region.
A list of prohibited ingredients incompatible with the geographic sub-region.
An ingredient vector comprising weighted characteristic ingredients.
An authenticity threshold value configurable by specificity level.

Further, the method may include receiving a meal plan request specifying a target geographic sub-regional cuisine identity. Further, for each meal in the meal plan, the method may include executing, by one or more processors, a tri-level validation comprising:
A first hard constraint validating the meal does not contain any prohibited ingredients.
A second hard constraint validating the meal contains all mandatory ingredients.
A soft constraint calculating a signature score based on characteristic ingredient presence and comparing to the authenticity threshold.

Further, the method may include rejecting meals that fail any constraint and programmatically invoking regeneration by a meal generation engine with updated constraint parameters.

Further, the geographic sub-regions comprise state-level or district-level cuisine distinctions within a national cuisine category.

Further, the authenticity threshold is configurable by specificity level comprising: lower thresholds for broad regional cuisines, and higher thresholds for state-level sub-regions.

Further, the signature score may be calculated by:
Extracting ingredients from the meal;
Comparing extracted ingredients against the characteristic ingredient vector;
Calculating a weighted sum of matching characteristic ingredients; and
Normalizing the sum to produce a score between 0.0 and 1.0.

Further, the cuisine signature framework is extensible to accommodate additional constraint types including religious dietary constraints.

In some embodiments, the present disclosure may describe a computer-implemented method for minimizing ingredient waste in multi-day meal planning through forward-chaining optimization. Further, the disclosed method may include a step of receiving a meal plan spanning N days where N is at least 2. Further, the disclosed method may include a step of aggregating total ingredient requirements across all N days. Further, the disclosed method may include a step of calculating optimal initial batch sizes for each ingredient based on aggregated requirements. Further, the disclosed method may include a step of assigning a unique chain identifier to each ingredient for tracking consumption lineage across days. Further, for each day D from 1 to N determining leftover quantities from previous day using the chain identifier, determining if leftovers are sufficient for day D's requirements, if leftovers are sufficient, consuming from leftover without new purchase, if leftovers are insufficient, calculating additional batch purchase needed, and updating the chain identifier with consumption and remaining leftover amount. Further, when leftover consumption causes nutritional deviation exceeding a predetermined threshold, distributing corrective adjustments across future days to maintain aggregate nutritional targets.

Further, the predetermined threshold for nutritional deviation comprises 50 kilocalories or 5 grams' protein.

Further, the calculating of the optimal batch sizes comprises considering standard packaging sizes available in grocery stores.

Further, the distributing of the corrective adjustments may include:
Calculating the deviation amount between actual and target nutrition.
Dividing the deviation by the number of remaining days.
Applying the correction to each remaining day's nutritional target.

Further, the disclosed the method achieves waste reduction of at least 30% compared to independent daily meal planning where each day is planned without consideration of previous or future days.

In some embodiments, the present disclosure describes a computer-implemented method for constraint-preserving bounded meal substitution. Further, the said method may include a step of receiving a user request to swap a meal in a generated meal plan, the request specifying a target meal to replace. Further, the said method may include a step of extracting nutritional properties of the target meal, including caloric content and macronutrient composition. Further, the said method may include a step of computing bounded nutritional ranges for replacement candidates. Further, the caloric bounds are set to ±50 kilocalories from the target meal's calories. Further, the protein bounds are set to ±5 grams from the target meal's protein content. Further, the said method may include a step of extracting all meal names from the active meal plan by iterating through all days in the planning cycle and collecting meal names from all meal types (breakfast, lunch, dinner, snacks), creating an exclusion list to prevent intra-plan repetition. Further, the said method may include a step of identifying replacement meal candidates from one or more sources selected from the group consisting of: (i) querying a meal database containing pre-existing recipes, and (ii) generating replacement meals using an artificial intelligence model. Further, the candidates satisfy the computed nutritional bounds, the user's dietary restrictions, the user's cuisine preferences, the user's allergen exclusions, non-membership in the meal name exclusion list created in the extracting step. Further, the said method may include a step of calculating weekly nutritional variance for each candidate to ensure selecting the candidate would not cause total weekly calories or protein to deviate by more than 1% from target weekly values. Further, the said method may include a step of filtering candidates to exclude those exceeding the weekly variance limit. Further, the said method may include a step of returning the filtered candidates as valid swap options, wherein each returned candidate is guaranteed to be different from all existing meals in the current plan. Further, upon user selection of a swap candidate, updating the meal plan with the selected replacement meal while preserving overall nutritional balance.

Further, the maximum per-meal calorie deviation is 50 kilocalories and the maximum per-meal protein deviation is 5 grams.

Further, the maximum weekly variance percentage is 1% of the weekly nutritional target.

Further, the similarity score is calculated by weighting calorie proximity and protein proximity, with calorie proximity weighted more heavily than protein proximity.

Further, the method enables user customization without requiring manual nutritional recalculation or expert intervention.

In some embodiments, the computer-implemented method for historical meal deduplication in personalized meal planning systems. Further, the disclosed method may include a step of generating a content-based fingerprint for each meal in a newly generated meal plan, wherein the fingerprint is derived from normalized ingredient names using a deterministic hash function. Further, the disclosed method may include a step of querying a user meal history database to retrieve fingerprints of meals served to the user within a predetermined lookback period. Further, the disclosed method may include a step of comparing fingerprints of meals in the newly generated plan against fingerprints in the user's meal history, wherein fingerprint comparison is performed using hash-based data structures to enable constant-time duplicate detection. Further, the disclosed method may include a step of detecting duplicate meals when a fingerprint match occurs between a new meal and a historical meal. Further, upon detecting duplicates, regenerating the meal plan by transmitting constraint updates to a meal generation engine to suppress ingredient combinations or content fingerprints corresponding to the detected duplication. Further, the disclosed method may include a step of implementing a retry mechanism with a maximum retry limit to prevent infinite loops. Further, the disclosed method may include a step of recording fingerprints of successfully generated non-duplicate meals in the user's meal history. Further, the disclosed method may include a step of maintaining a temporal constraint wherein meals are considered duplicates only if served within the predetermined lookback period, with the period configurable by the user or system administrator. Further, the disclosed method may include a step of integrating with the bounded meal swap system such that: (i) meal swap candidates are filtered against the user's meal history to prevent suggesting meals recently served (inter-plan deduplication); and (ii) swapped meals are immediately recorded in the meal history database with the corresponding fingerprints, preventing reappearance in future generated plans or subsequent swap suggestions.

In some embodiments, the present disclosure may describe a fingerprint generation system for meal deduplication. Further, the system may include an ingredient normalization module that converts ingredient names and quantities to standardized lowercase units. Further, the system may include a content aggregation module that combines meal name, normalized ingredients, caloric content, protein content, and preparation instructions into a deterministic string representation. Further, the system may include a hash generation module that applies a bitwise hash function to said deterministic string to produce an 8-character hexadecimal fingerprint. Further, the said fingerprint is deterministic such that identical meal content always produces identical fingerprints regardless of input order or formatting variations.

In some embodiments, the present disclosure may describe a temporal deduplication system for personalized meal planning. Further, the disclosed system may include a database storing user meal history with timestamp granularity. Further, the disclosed system may include a configurable lookback window (default 14 days) for retrieving recent meals. Further, the disclosed system may include a retry mechanism that attempts meal plan regeneration up to a configurable maximum threshold (e.g., 3 attempts). Further, the disclosed system may include a fingerprint comparison module that checks candidate meals against said recent meal history using Set-based data structures for O (1) lookup complexity. Further, the disclosed system may include a persistence layer that records successfully generated meals with the corresponding fingerprints, meal types, and nutritional metadata for future deduplication checks.

Further, the said content-based fingerprint generation includes alphabetical sorting of normalized ingredients to ensure deterministic ordering independent of original recipe formatting.

Further, the said maximum retry threshold is configurable based on plan generation frequency.

Further, the said ingredient normalization module standardizes measurement units across cultural contexts, converting units to canonical representations.

Further, the said lookback window duration is configurable based on meal plan frequency.

Further, the collision detection includes nutritional tolerance thresholds, treating meals with caloric differences under 5% and protein differences under 2 g as duplicates.

Further, the said retry mechanism includes a delay interval between attempts to allow for AI model response diversity.

In some embodiments, the present disclosure describes the following advantages of the disclosed system over prior art:

### Advantage 1: Geographic Sub-Regional Specificity

Prior Art: Generic ‘Indian’ or ‘South Indian’ categories without enforcement mechanism.
Disclosed system (Implemented): Geographic sub-regional specificity (Kerala vs Tamil Nadu vs Punjab) with tri-level validation comprising prohibited ingredients, mandatory ingredients, and signature scoring.
Future Extension (Contemplated): Framework extensible to religious/cultural sub-groups (Brahmin vegetarian, Jain) using same algorithmic approach.
Impact: Users receive culturally authentic meals matching the user's geographic heritage, with architecture supporting future religious constraint validation.

### Advantage 2: Waste Reduction

Prior Art: Independent daily planning resulting in significant ingredient waste.
Disclosed system: Forward-chaining optimization with chain ID tracking and predictive batch sizing.
Impact: Significant waste reduction through batch optimization, with cost savings and environmental benefit.

### Advantage 3: Safe User Customization

Prior Art: Unrestricted swaps (violating goals), prohibited swaps (poor UX), or manual recalculation (requiring experts). Swap suggestions may repeat meals already in the current plan.
Disclosed system: Bounded swaps with multi-dimensional constraint enforcement (calories, protein, cuisine, dietary restrictions, intra-plan deduplication, weekly variance). Intra-plan deduplication extracts all meal names from the current plan to create an exclusion list, ensuring swap suggestions are different from all existing meals in the active week.
Impact: User empowerment without expert intervention while preserving nutritional and cultural goals. Guaranteed variety within the planning window.

### Advantage 4: Meal Variety Through Deduplication

Prior Art: Stateless AI generation resulting in meal repetition across weeks.
Disclosed system: Content-based fingerprinting with temporal tracking and intelligent retry logic.
Impact: Reliably detects duplicate meals, increased perceived AI creativity, reduced user fatigue.

Further, the disclosed system is applicable to:
Meal Planning SaaS Platforms: Consumer meal planning applications requiring cultural authenticity and variety.
Food Delivery Services: Subscription meal kit services optimizing ingredient utilization and preventing menu fatigue.
Healthcare Nutrition Apps: Clinical nutrition programs requiring constraint enforcement and longitudinal tracking.
Enterprise Cafeteria Systems: Corporate meal planning with waste reduction goals and dietary diversity.
Grocery Delivery Optimization: Ingredient purchasing optimization across multi-day periods with temporal user preferences.

Further, the present disclosure provides a method of facilitating non-repetitive meal planning. Further, the method may include receiving, using a communication device, a meal request data from one or more client devices. Further, the method may include receiving, using the communication device, a meal history data from the one or more client devices. Further, the method may include storing, using a storage device, the meal history data. Further, the method may include generating, using a processing device, a candidate meal data in response to the meal request data. Further, the method may include computing, using the processing device, a meal fingerprint data for the candidate meal data based on a normalized ingredient data associated with the candidate meal data. Further, the method may include comparing, using the processing device, the meal fingerprint data with the meal history data. Further, the method may include identifying, using the processing device, a duplication based on the comparing of the meal fingerprint data with the meal history data. Further, the method may include determining, using the processing device, a non-duplicate meal data based on the identifying of the duplication. Further, the method may include storing, using the storage device, the non-duplicate meal data. Further, the method may include transmitting, using the communication device, the non-duplicate meal data to the one or more client devices.

Further, in some embodiments, the computing of the meal fingerprint data may include extracting an ingredient data from the candidate meal data. Further, the computing of the meal fingerprint data may include normalizing the ingredient data by converting ingredient name to a canonical textual form. Further, the computing of the meal fingerprint data may include ordering the normalized ingredient data according to a deterministic sequence. Further, the computing of the meal fingerprint data may include generating an ordered ingredient data based on the ordering. Further, the generating of the meal fingerprint data may be further based on the ordered ingredient data.

Further, in some embodiments, the computing of the meal fingerprint data may include aggregating a meal content data comprising an ingredient data and a nutritional data associated with the candidate meal data. Further, the computing of the meal fingerprint data may include transforming the meal content data into a deterministic string representation. Further, the computing of the meal fingerprint data may include hashing the deterministic string representation. Further, the computing of the meal fingerprint data may be further based on the hashing of the deterministic string representation.

Further, in some embodiments, the method may include retrieving, using the storage device, a historical meal dataset associated with a predefined temporal window. Further, in some embodiments, the method may include filtering, using the processing device, the meal history data to include only meal fingerprint data stored within the predefined temporal window.

Further, in some embodiments, the comparing of the meal fingerprint data with the meal history data may include loading the meal history data into a hash-based lookup structure. Further, the comparing of the meal fingerprint data with the meal history data may include evaluating a membership of the meal fingerprint data within the hash-based lookup structure. Further, the comparing of the meal fingerprint data with the meal history data may be further based on the evaluating of the member of the meal fingerprint data within the hash-based lookup structure.

Further, in some embodiments, the method may include detecting, using the processing device, a duplication condition when the meal fingerprint data matches the meal history data. Further, in some embodiments, the method may include initiating, using the processing device, a regeneration control operation in response to the duplication condition. Further, in some embodiments, the method may include limiting, using the processing device, a number of regeneration attempts based on a predefined retry threshold.

Further, in some embodiments, the computing of the meal fingerprint data may include extracting a nutritional data associated with the candidate meal data. Further, the computing of the meal fingerprint data may include incorporating the nutritional data into the meal fingerprint data for differentiating nutritionally distinct meals.

Further, in some embodiments, the storing of the non-duplicate meal data may include associating the non-duplicate meal data with a user identifier. Further, the storing of the non-duplicate meal data may include persisting the non-duplicate meal data and the meal fingerprint data in a user-specific meal history record.

Further, in some embodiments, the determining of the non-duplicate meal data may include evaluating a hash collision condition based on a predefined collision tolerance parameter. Further, the determining of the non-duplicate meal data may include confirming the non-duplicate meal data when the hash collision condition may be not satisfied.

Further, in some embodiments, the comparing of the meal fingerprint data with the meal history data may include identifying a user-scoped meal history subset based on a user identifier. Further, the comparing of the meal fingerprint data with the meal history data may include restricting the comparing to the user-scoped meal history subset.

Further, the present disclosure provides a system of facilitating non-repetitive meal planning. Further, the system may include a communication device. Further, the communication device may be configured for receiving a meal request data from one or more client devices. Further, the communication device may be configured for receiving a meal history data from the one or more client devices. Further, the communication device may be configured for transmitting the non-duplicate meal data to the one or more client devices. Further, the system may include a processing device communicatively coupled with the communication device. Further, the processing device may be configured for generating a candidate meal data in response to the meal request data. Further, the processing device may be configured for computing a meal fingerprint data for the candidate meal data based on a normalized ingredient data associated with the candidate meal data. Further, the processing device may be configured for comparing the meal fingerprint data with the meal history data. Further, the processing device may be configured for identifying a duplication based on the comparing of the meal fingerprint data with the meal history data. Further, the processing device may be configured for determining a non-duplicate meal data based on the identifying of the duplication. Further, the system may include a storage device communicatively coupled with the processing device. Further, the storage device may be configured for storing the meal history data. Further, the storage device may be configured for storing the non-duplicate meal data.

Further, in some embodiments, the computing of the meal fingerprint data may include extracting an ingredient data from the candidate meal data. Further, the computing of the meal fingerprint data may include normalizing the ingredient data by converting ingredient name to a canonical textual form. Further, the computing of the meal fingerprint data may include ordering the normalized ingredient data according to a deterministic sequence. Further, the computing of the meal fingerprint data may include generating an ordered ingredient data based on the ordering. Further, the generating of the meal fingerprint data may be further based on the ordered ingredient data.

Further, in some embodiments, the computing of the meal fingerprint data may include aggregating a meal content data comprising an ingredient data and a nutritional data associated with the candidate meal data. Further, the computing of the meal fingerprint data may include transforming the meal content data into a deterministic string representation. Further, the computing of the meal fingerprint data may include hashing the deterministic string representation. Further, the computing of the meal fingerprint data may be further based on the hashing of the deterministic string representation.

In some embodiments, the storage device may be further configured for retrieving a historical meal dataset associated with a predefined temporal window. Further, the processing device may be further configured for filtering the meal history data to include only meal fingerprint data stored within the predefined temporal window.

Further, in some embodiments, the comparing of the meal fingerprint data with the meal history data may include loading the meal history data into a hash-based lookup structure. Further, the comparing of the meal fingerprint data with the meal history data may include evaluating a membership of the meal fingerprint data within the hash-based lookup structure. Further, the comparing of the meal fingerprint data with the meal history data may be further based on the evaluating of the member of the meal fingerprint data within the hash-based lookup structure.

Further, in some embodiments, the processing device may be further configured for detecting a duplication condition when the meal fingerprint data matches the meal history data. Further, the processing device may be further configured for initiating a regeneration control operation in response to the duplication condition. Further, the processing device may be further configured for limiting a number of regeneration attempts based on a predefined retry threshold.

Further, in some embodiments, the computing of the meal fingerprint data may include extracting a nutritional data associated with the candidate meal data. Further, the computing of the meal fingerprint data may include incorporating the nutritional data into the meal fingerprint data for differentiating nutritionally distinct meals.

Further, in some embodiments, the storing of the non-duplicate meal data may include associating the non-duplicate meal data with a user identifier. Further, the storing of the non-duplicate meal data may include persisting the non-duplicate meal data and the meal fingerprint data in a user-specific meal history record.

Further, in some embodiments, the determining of the non-duplicate meal data may include evaluating a hash collision condition based on a predefined collision tolerance parameter. Further, the determining of the non-duplicate meal data may include confirming the non-duplicate meal data when the hash collision condition may not be satisfied.

Further, in some embodiments, the comparing of the meal fingerprint data with the meal history data may include identifying a user-scoped meal history subset based on a user identifier. Further, the comparing of the meal fingerprint data with the meal history data may include restricting the comparing to the user-scoped meal history subset.

In some embodiments, the disclosed system provides inherent technical improvements to the field of computational meal planning systems, and more particularly to data-driven content deduplication, personalization engines, and server-side generation workflows implemented using software-as-a-service architectures. The given improvements arise from non-conventional data processing techniques that address specific technical limitations in existing systems, rather than from abstract user preferences or business rules.

In some embodiments, a first inherent technical improvement may relate to the use of deterministic, content-based fingerprint generation for structured food data, which improves the technology of duplicate detection in server-side recommendation systems. Conventional systems typically rely on identifier-based matching, string comparison, or name similarity, which fails when semantically identical meals are expressed using different textual representations or minor formatting variations. In contrast, the disclosed system may compute a fingerprint derived from normalized ingredient data, nutritional data, and preparation-related content, thereby transforming heterogeneous meal representations into a deterministic digital signature. The given fingerprinting process may be implemented using bitwise hash functions, rolling hash algorithms, or other deterministic hashing techniques that operate over normalized and ordered content vectors. By ensuring that identical meal content yields identical fingerprints regardless of input order or formatting, the system may improve the accuracy and reliability of duplicate detection technology used in large-scale SaaS platforms. The given improvement directly enhances the underlying data integrity and comparison technology employed by recommendation engines.

In some embodiments, another inherent technical improvement may involve normalization and canonicalization of structured ingredient data prior to fingerprint computation, thereby improving the technology of semantic data comparison in distributed systems. Ingredient data received from heterogeneous sources may vary in spelling, unit representation, casing, or ordering, which conventionally leads to false negatives during comparison. The disclosed system may normalize ingredient data by converting ingredient names to a canonical textual form, standardizing measurement units, and ordering ingredients deterministically. The given normalized representation may then be used as a stable input to hashing or comparison functions. Such normalization techniques improve the robustness of semantic equivalence detection in structured data processing systems and reduce computational noise introduced by non-semantic variation.

In some embodiments, an inherent technical improvement may relate to temporal scoping of deduplication operations using configurable lookback windows, thereby improving the technology of state management in stateless SaaS generation systems. Traditional AI-driven generation systems operate in a stateless manner, lacking memory of prior outputs, which leads to repeated content generation and inefficiencies. The disclosed system may retrieve and filter historical fingerprint data based on a predefined temporal window, such as a rolling number of days or generation cycles, and may perform deduplication only within that window. The given approach improves the balance between computational cost and functional effectiveness, enabling bounded statefulness without requiring indefinite historical storage. The underlying technology improved is server-side session memory management and temporal data indexing.

In some embodiments, the disclosed system may inherently improve computational efficiency in duplicate detection by employing hash-based data structures for constant-time lookup operations. Rather than performing linear scans or expensive similarity computations across historical datasets, the system may load fingerprint data into hash sets or equivalent lookup structures. Membership evaluation may then be performed in constant time, significantly reducing latency during meal plan generation. The given approach improves the efficiency of large-scale comparison algorithms used in SaaS platforms and enhances system scalability under high user concurrency.

In some embodiments, an inherent technical improvement may arise from controlled regeneration logic triggered by detected duplication, thereby improving the technology of iterative content generation pipelines. When a duplication condition is detected, the system may initiate regeneration of candidate meal data under modified constraints or parameters, rather than returning duplicate content or failing silently. The given regeneration process may be governed by retry thresholds, random seed variation, or constraint suppression logic, ensuring deterministic termination while maximizing content diversity. Such control-flow mechanisms improve the reliability and predictability of automated generation systems.

In some embodiments, the disclosed system may inherently improve the robustness of duplicate detection by incorporating nutritional data into fingerprint generation. Two meals that share similar ingredients but differ materially in nutritional composition may be treated as distinct when nutritional data is included in the fingerprint. The given approach improves the fidelity of content differentiation in systems where nutritional precision is technically significant, thereby enhancing the underlying data modeling technology used in nutrition-aware applications.

In some embodiments, persistent storage of validated, non-duplicate fingerprints may provide an inherent technical improvement to adaptive learning behavior in SaaS systems. By storing fingerprint data associated with generated meals, the system may incrementally improve future duplicate prevention without retraining machine learning models or reprocessing historical content. The given approach improves the efficiency of long-term personalization mechanisms and reduces redundant computation in recurring generation tasks.

In some embodiments, user-scoped deduplication may inherently improve the technology of multi-tenant SaaS architectures by isolating fingerprint comparison to user-specific datasets. Rather than maintaining a global deduplication state, which may introduce unnecessary constraints or collisions across users, the system may restrict comparison to fingerprints associated with a specific user identifier, improving data isolation, reduces false positives, and enhances scalability in multi-user environments.

In some embodiments, a technical improvement may include adaptive fingerprint weighting mechanisms that dynamically adjust which meal attributes contribute most strongly to fingerprint generation. For example, ingredient data may be weighted more heavily than preparation text in some contexts, while nutritional data may be emphasized in others. Such weighting may be configured based on user goals or system performance metrics. The given feature improves the technology of content hashing and similarity modeling by enabling context-aware fingerprint sensitivity.

In some embodiments, a technical improvement may include collision mitigation mechanisms that detect and resolve potential hash collisions without abandoning deterministic hashing. The system may perform secondary validation steps, such as comparing normalized ingredient vectors or nutritional thresholds, when a hash match is detected, improving the reliability of hash-based deduplication technology while preserving computational efficiency.

In some embodiments, a technical improvement may include incremental fingerprint evolution, wherein fingerprint schemas may be versioned over time. The system may generate fingerprints using different schema versions and may store version metadata alongside fingerprint data, allowing backward compatibility and controlled evolution of fingerprint logic without invalidating historical data, improving the technology of schema evolution and backward compatibility in persistent SaaS systems.

In some embodiments, a technical improvement may include distributed fingerprint caching across server instances. Frequently accessed fingerprint data may be cached in memory or distributed cache layers to reduce database access latency, improving the technology of distributed system performance and load balancing in high-throughput SaaS deployments.

In some embodiments, a technical improvement may include probabilistic regeneration prioritization, wherein the system may analyze historical regeneration outcomes to predict which constraint modifications are most likely to yield non-duplicate content, reducing unnecessary regeneration attempts and improves the efficiency of automated content pipelines. The technology improved is probabilistic control-flow optimization in server-side generation systems.

Further, the given technical improvements demonstrate that the disclosed system advances multiple specific technologies, including structured data normalization, deterministic fingerprinting, temporal state management, hash-based comparison algorithms, and scalable SaaS content generation architectures. The improvements are rooted in concrete data processing techniques and system-level optimizations, rather than abstract concepts, and may be implemented in various combinations without departing from the scope of the disclosed system.

In some embodiments, the disclosed systems and methods may provide a technical improvement in the field of computer-implemented meal planning by introducing deterministic content-level deduplication mechanisms that operate on normalized multi-dimensional data representations rather than on superficial metadata or heuristic similarity scores. A technical problem addressed in the given context is that existing systems frequently rely on high-level attributes such as recipe names, categories, or user ratings, which do not reliably identify substantive equivalence between distinct data objects. In some embodiments, the given problem may be addressed by transforming heterogeneous meal-related data into a normalized canonical form prior to comparison, thereby enabling deterministic equivalence detection using content-based hashing techniques. The given approach may improve the underlying technology of data deduplication by enabling constant-time equality evaluation using hash-based data structures, rather than probabilistic or threshold-based similarity matching.

In some embodiments, the disclosed systems may improve the technical field of computational constraint validation by introducing a multi-stage validation pipeline that enforces hierarchical constraints using explicit rule execution rather than single-pass filtering. A technical problem arises when multiple constraint types—such as exclusionary constraints, mandatory inclusion constraints, and weighted scoring constraints—must be evaluated simultaneously, often resulting in ambiguous or inconsistent validation outcomes. In some embodiments, the given problem may be addressed by decomposing validation into ordered computational stages, each producing an intermediate validation state. Such staged validation may enable deterministic failure detection, early rejection of invalid data objects, and improved computational efficiency by reducing unnecessary downstream processing, improving the technology of rule-based data validation engines by enabling predictable execution flow and reduced computational overhead.

In some embodiments, the disclosed techniques may improve the technical field of time-series data planning by enabling forward-projected resource utilization modeling across discrete planning intervals. A technical problem in the given domain is that many planning systems operate on isolated time slices and lack mechanisms for forecasting cumulative resource usage across future intervals. In some embodiments, the given problem may be addressed by aggregating resource requirement data prior to temporal assignment, thereby enabling predictive modeling of usage patterns. The said approach may improve planning algorithms by enabling preemptive detection of overuse, underuse, or imbalance across a planning horizon.

In some embodiments, the disclosed systems may improve computational inventory modeling by introducing identifier-based chaining of resource consumption events across time. A technical problem exists in tracking partial consumption of resources across multiple operations without duplicative accounting or loss of state. In some embodiments, the said issue may be addressed by assigning persistent chain identifiers to resource batches and decrementing associated state values as consumption events occur. The given technique may improve the technology of stateful resource tracking by enabling accurate carry-forward of partially consumed quantities across multiple computational cycles.

In some embodiments, the disclosed systems may improve nutritional modeling algorithms by dynamically redistributing deviations caused by upstream constraints across remaining planning intervals. A technical problem addressed here is that static nutritional targets often fail when upstream decisions alter available resources or consumption patterns. In some embodiments, the said problem may be addressed by computing cumulative deviation values and algorithmically redistributing those deviations across future intervals, improving the technology of constraint-balancing algorithms by enabling adaptive recalibration rather than rigid enforcement.

In some embodiments, the disclosed systems may improve the field of structured data normalization by introducing deterministic ordering and canonicalization of multi-source data prior to transformation. A technical problem arises when semantically identical data objects are represented differently due to ordering, formatting, or source-specific variation. In some embodiments, the said problem may be addressed by enforcing deterministic ordering rules prior to transformation, thereby ensuring stable and repeatable transformation outputs, may improving the technology of data serialization and hashing by reducing false negatives in equivalence detection.

In some embodiments, the disclosed systems may further improve the technology of hash-based data comparison by implementing configurable temporal scoping mechanisms that limit comparison operations to defined historical windows. A technical problem addressed here is the unbounded growth of comparison datasets, which may degrade performance over time. In some embodiments, the said problem may be addressed by restricting lookup operations to time-bounded datasets, thereby improving computational efficiency while preserving functional correctness.

In some embodiments, the disclosed systems may improve automated generation systems by dynamically modifying generation constraints in response to detected duplication events. A technical problem arises when generation systems repeatedly produce equivalent outputs despite prior rejection. In some embodiments, the said problem may be addressed by identifying constraint dimensions associated with rejected outputs and excluding those dimensions from subsequent generation cycles, improving the technology of automated content generation by reducing repetitive failure loops and increasing convergence efficiency.

In some embodiments, the disclosed systems may improve the robustness of multi-user SaaS platforms by enforcing strict user-scoped isolation of historical data during comparison operations. A technical problem exists when shared datasets introduce cross-user interference or unintended correlation. In some embodiments, the said problem may be addressed by restricting comparison and lookup operations to user-specific data partitions, improving the technology of multi-tenant data isolation by preventing cross-context contamination.

In some embodiments, the disclosed systems may improve persistence-layer efficiency by storing transformation outputs alongside generation metadata in a unified record structure. A technical problem arises when derived data must be recomputed due to a lack of persistent association. In some embodiments, the said problem may be addressed by persisting derived identifiers together with timestamps and user identifiers, improving the technology of data caching and retrieval by reducing recomputation and improving traceability.

In some embodiments, the disclosed techniques may improve system scalability by enabling constant-time duplicate detection using hash-based lookup structures rather than linear or quadratic comparison algorithms. A technical problem addressed here is that similarity-based comparison methods often scale poorly with dataset size. In some embodiments, the said problem may be addressed by transforming comparison into a deterministic equality check, improving the technology of large-scale recommendation systems by enabling predictable performance under increasing load.

In some embodiments, a computer-implemented meal planning service is executed by an online server including one or more processors, non-transitory memory, and one or more network interfaces that exchange meal-planning request records and meal-plan results with a client device. The server maintains and/or accesses structured data objects including (i) candidate meal data (e.g., ingredient, nutrition, and preparation-instruction fields), (ii) cuisine signature data for a selected geographic (sub-) regional cuisine identity (e.g., a list of prohibited ingredients, a list of mandatory ingredients, and cuisine-specific weight parameters for characteristic ingredients), and (iii) user meal history data (e.g., prior meal fingerprints with timestamps). To evaluate cultural authenticity with objective boundaries, the server performs a tri-level validation that (a) confirms absence of prohibited ingredients, (b) confirms presence of mandatory ingredients, and (c) computes a cuisine signature score from a weighted characteristic ingredient representation (for example, by mapping canonical ingredient names and quantities to a canonical ingredient vector and computing a weighted sum/dot-product), and compares the score to a configurable authenticity threshold that may be selected based on a stored geographic specificity level. For non-repetition, the server forms normalized data for a meal by deterministically normalizing and ordering ingredient, nutrition, and instruction fields (e.g., canonical text, canonical units, deterministic ordering), concatenating the normalized fields into a deterministic content string, and applying a deterministic hash to generate a content-based meal fingerprint; fingerprints are efficiently compared within a predefined temporal lookback window (e.g., via a hash-based lookup structure). For multi-day planning, ingredient usage may be projected by aggregating ingredient quantity values across selected meals to generate ingredient requirement data, from which batch quantities and remaining quantities may be tracked; if remaining quantities cause a measurable nutritional deviation relative to a defined nutritional target (e.g., calories/macros/micros), the server rebalances remaining-day targets by distributing the deviation across remaining days. When duplication is detected, the server generates a replacement meal by applying constraints that both satisfy the tri-level validation and exclude the duplicated ingredient combination(s), thereby producing an objectively “authentic” and non-duplicate meal plan while improving computational efficiency through deterministic normalization, hashing, and constant-time membership checks.

In some embodiments, the normalized data comprises a set of deterministically normalized fields derived from the meal (i.e., a candidate meal, an authentic candidate meal, a non-duplicate validated meal, etc.) including normalized ingredient data, normalized nutritional data, and normalized preparation instruction data. For example, the processing device generates the normalized ingredient data by converting ingredient names to canonical textual forms, standardizing ingredient units to canonical units, and ordering ingredient entries in a deterministic sequence; the processing device likewise generates the normalized nutritional data and the normalized preparation instruction data using predetermined canonicalization and ordering rules. The processing device may form a deterministic content string by concatenating the normalized ingredient data, the normalized nutritional data, and the normalized preparation instruction data in a fixed format, such that applying a deterministic hash function to the deterministic content string yields a repeatable content-based meal fingerprint data for the meal.

In some embodiments, the processing device projects ingredient usage across the multi-day planning duration by aggregating ingredient quantity data across the multi-day planning duration for ingredients appearing in the plurality of authentic candidate meal data (or plurality of non-duplicate validated meal data). The processing device then generates ingredient requirement data based on the aggregated ingredient quantity data, where the ingredient requirement data identifies each ingredient and an associated required quantity for the multi-day planning duration. The processing device may use the ingredient requirement data to generate ingredient batch data representing a batch quantity for an ingredient identified in the ingredient requirement data, including initializing the batch quantity based on a determined batch purchase quantity.

In some embodiments, “authentic candidate meal data” refers to candidate meal data that satisfies a defined tri-level validation against cuisine signature data corresponding to a geographic sub-regional cuisine identity. The cuisine signature data may define (i) one or more prohibited ingredients, (ii) one or more mandatory ingredients, and (iii) one or more cuisine-specific weight parameters used to compute a cuisine signature score from weighted characteristic ingredient data derived from the candidate ingredient data; the cuisine signature score is compared to a configurable authenticity threshold to determine pass/fail. In some embodiments, a “nutritional deviation” is a computed difference between a nutritional target and an achieved nutritional value attributable to ingredient usage and remaining quantity value (e.g., per nutrient, per day, or cumulatively across days), and the processing device rebalances the nutritional target across remaining days by distributing the deviation according to a defined allocation rule. In some embodiments, an “at least one constraint” used to generate replacement candidate meal data is an explicit, machine-evaluable rule (e.g., ingredient inclusion/exclusion, ingredient combination exclusion, nutrient bounds, preparation constraints, or cost/availability constraints) that is applied such that the replacement candidate meal data both satisfies the tri-level validation and avoids duplication within the predefined temporal lookback window.

In some embodiments, the techniques described herein relate generally to generating and transmitting meal plan data responsive to meal planning request data, including selecting meals, validating meals, and reducing duplication based on user meal history data. Any references in this description to facilitating “culturally-authentic” meal planning or “non-repetitive” meal planning are provided as descriptive context for certain embodiments and do not limit the scope of the disclosed techniques, which may be applied to meal planning across a variety of cuisines, dietary patterns, and planning durations, using the same operational steps and data structures described herein.

In some embodiments, the communication device includes one or more physical communication interfaces (e.g., a network interface controller, modem, transceiver, or wireless adapter) that enable receipt and transmission of data between an online server and a client device over one or more networks, and may include associated drivers and protocol stacks stored in memory. In some embodiments, the processing device includes one or more physical processors (e.g., a CPU, GPU, DSP, or microcontroller) that execute instructions to perform the operations described herein, including selecting candidate meal data, executing tri-level validation, and generating content-based meal fingerprint data. In some embodiments, the storage device includes one or more physical non-transitory storage media (e.g., RAM, ROM, flash memory, or database storage) that store cuisine signature data, user meal history data, ingredient batch data, and meal plan data. The term “configured to” as used herein denotes that the identified device includes the recited structure and/or executes stored instructions to perform the recited operations.

In some embodiments, the non-duplication operations improve computer performance by using deterministic normalization, deterministic content strings, and a deterministic hash function to generate a compact content-based meal fingerprint data that supports rapid duplication detection. For example, the processing device loads a plurality of historical content-based meal fingerprint data within a predefined temporal lookback window into a hash-based lookup structure, enabling efficient membership evaluation relative to naive pairwise comparisons. In some embodiments, these mechanisms reduce processor cycles, memory accesses, and/or network transfers associated with repeated meal comparisons, and improve consistency of duplication detection across heterogeneous ingredient naming and unit representations through canonicalization and deterministic ordering.

In some embodiments, the processing device computes the cuisine signature score by transforming candidate ingredient data into canonical ingredient name data and canonical ingredient quantity data, constructing a canonical ingredient vector, applying cuisine-specific weight parameters from cuisine signature data to produce a weighted representation, and computing a scalar score (e.g., weighted sum or dot product) that is compared to the configurable authenticity threshold. In some embodiments, the predefined temporal lookback window is represented as a time interval preceding receipt of the meal planning request data, and user meal history data stores historical content-based meal fingerprint data with timestamps to enable time-scoped retrieval. When duplication is identified, the processing device generates replacement candidate meal data by applying at least one constraint that enforces satisfaction of the tri-level validation and modifies the candidate space to avoid the duplicated fingerprint and/or the duplicated ingredient combination that produced it. In some embodiments, the ingredient chain identifier links ingredient batch data to consumption quantity updates across days, and the processing device computes remaining quantity value and updates or reallocates the nutritional target across remaining days using a defined rule (e.g., distributing cumulative nutritional deviation proportionally or evenly across remaining days) to maintain adherence to nutrient objectives while respecting remaining ingredient quantities.

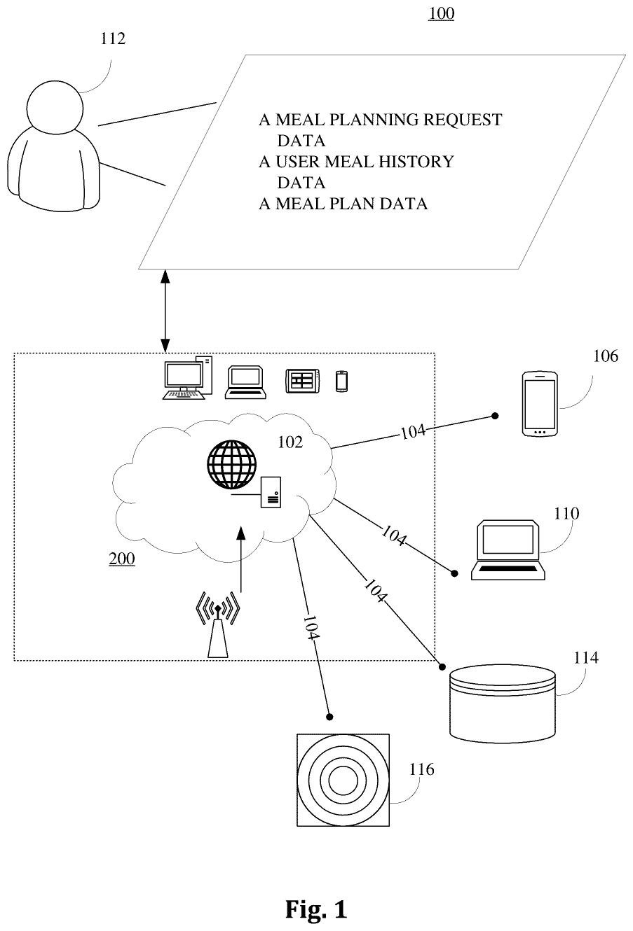

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, users, administrators, service providers, service consumers, and so on. Accordingly, in some instances, electronic devices operated by the one or more relevant parties may be in communication with the platform.

A user 112, such as the one or more relevant parties, may access online platform 100 through a web-based software application or browser. The web-based software application may be embodied as, for example, but not limited to, a website, a web application, a desktop application, and a mobile application compatible with a computing device 200.

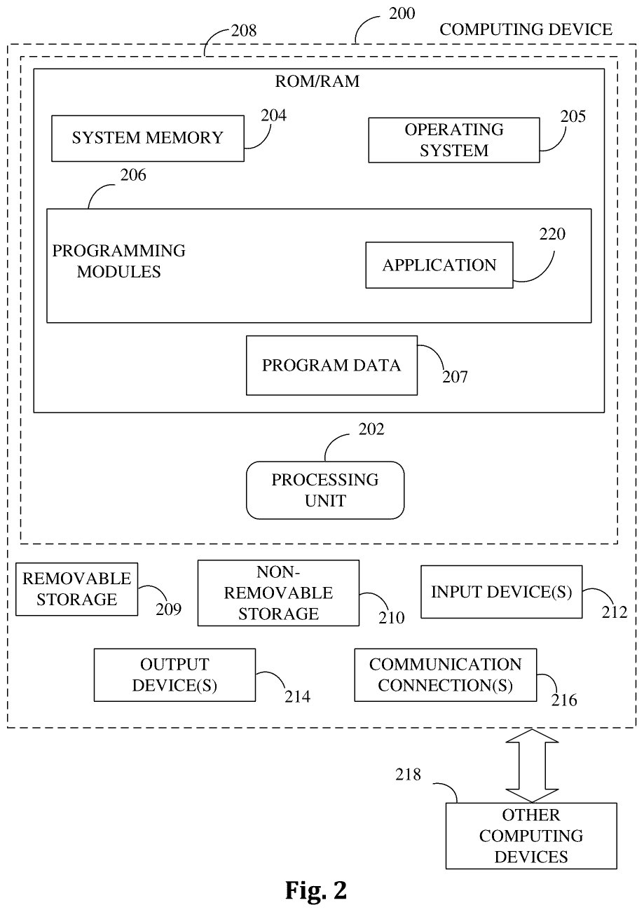

With reference to FIG. 2, a system consistent with an embodiment of the disclosure may include a computing device or a cloud service, such as a computing device 200. In a basic configuration, the computing device 200 may include at least one processing unit 202 and a system memory 204. Further, the at least one processing unit 202 may include a processor. Moreover, the processor may be and/or may include general-purpose processors, specialized processors, machine learning accelerators, multi-core processors, vector processors, digital signal processors, tensor accelerators, neural accelerators, graphics engines, or various kinds of specialized integrated circuits including FPGAs, ASICs, ASSPs, SoCs, and CPLDs. Depending on the configuration and type of the computing device 200, the system memory 204 may comprise, but is not limited to, a volatile memory (e.g., random-access memory (RAM)), non-volatile memory (e.g., read-only memory (ROM)), a flash memory, or any combination. System memory 204 may include an operating system 205, one or more programming modules 206, and a program data 207. Operating system 205, for example, may be suitable for controlling computing device 200's operation. In one embodiment, the one or more programming modules 206 may include image-processing modules, machine learning modules, etc. Furthermore, embodiments of the disclosure may be practiced in conjunction with a graphics library, other operating systems, or any other application program, and are not limited to any particular application or system. This basic configuration is illustrated in FIG. 2 by those components within a dashed line 208.

Computing device 200 may have additional features or functionality. For example, the computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage). Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of the computing device 200. Computing device 200 may also have input device(s) 212, such as a keyboard, a mouse, a pen, a sound input device, a touch input device, a location sensor, a camera, a biometric sensor, etc. Output device(s) 214, such as a display, speakers, a printer, etc., may also be included. Further, the input device(s) 212 and the output device(s) 214 may suitable for multimodal interaction with a user. The aforementioned devices are examples, and others may be used.

Computing device 200 may also contain a communication connection 216 that may allow device 200 to communicate with other computing devices 218, such as over a network in a distributed computing environment, for example, an intranet or the Internet. Further, the communication connection may be and/or may include a communication interface, a network interface, etc. Communication connection 216 is one example of communication media. Communication media may typically be embodied by computer-readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein, may include both storage media and communication media.

As stated above, a number of program modules and data files may be stored in the system memory 204, including the operating system 205. While executing on the at least one processing unit 202, the one or more programming modules 206 (e.g., application 220 such as a media player) may perform processes including, for example, one or more stages of methods, algorithms, systems, applications, servers, and databases as described above. The aforementioned process is an example, and the at least one processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computing device and/or computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations, such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage medium readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example, but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Further, the computer-readable medium may be and/or may include one or more non-transitory computer readable media. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently, or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

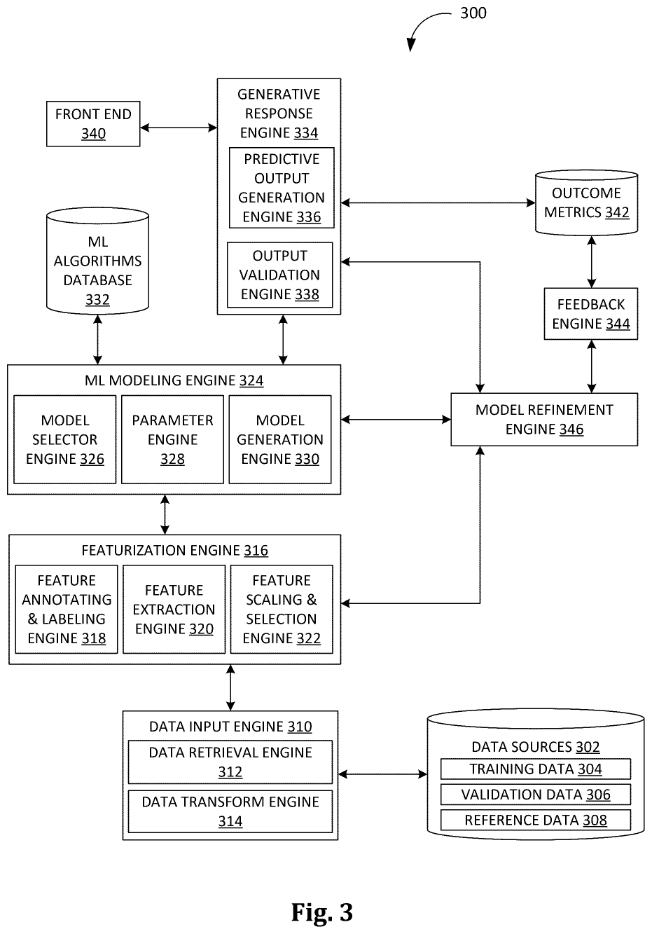

FIG. 3 is a block diagram illustrating a machine-learning system 300 for implementing various embodiments of this disclosure, in accordance with some embodiments. Although the disclosed machine-learning system 300 depicts particular system components and an arrangement of such components, the given depiction is to facilitate a discussion of the present technology and should not be considered limiting unless specified in the appended claims. For, example some components that are illustrated as separate, may be combined with other components and some components may be divided into separate components.

Accordingly, the machine-learning system 300 may include a plurality of interrelated modules and engines configured to implement a machine-learning pipeline. Further, the machine-learning system 300 may include a data sources module 302 that is made up of a training data repository 304, a validation data repository 306, and a reference data repository 308, each repository being configured to store respective classes of input records and reference information. Further, the machine-learning system 300 may include a data input engine 310 configured to receive data from the data sources module 302. Further, the data input engine 310 may include a data retrieval engine 312 configured to access and ingest data from the repositories (304, 306, 308), and a data transform engine 314 configured to perform initial normalization, parsing, and format conversion on the ingested data. Further, the data input engine 310 may be implemented on a computing device.

Further, the machine-learning system 300 may include a featurization engine 316 configured to prepare temporal and predictive representations of transformed data. Further, the featurization engine 316 may include a feature annotating & labeling engine 318 for applying labels and annotations to data instances, a feature extraction engine 320 for deriving feature vectors and candidate predictors, and a feature scaling & selection engine 322 for performing numerical scaling, dimensionality reduction, and selection of salient features. Further, the featurization engine 316 may be implemented on the computing device.

Further, the machine-learning system 300 may include a machine learning (ML) modeling engine 324 configured to construct predictive models from selected features. Further, the ML modeling engine 324 may include a model selector engine 326 for selecting among candidate model classes, a parameter engine 328 for determining and tuning hyper parameters, and a model generation engine 330 for instantiating and training model artifacts according to selected architectures and parameters. Further, the machine-learning system 300 may include an ML algorithms database 332 configured to store algorithmic implementations, model templates and associated metadata and to be accessible by components of the ML modeling engine 324. Further, the ML modeling engine 324 may be implemented on the computing device.

Further, the machine-learning system 300 may include a generative response engine 334 configured to produce user-facing outputs based on the trained models. Further, the generative response engine 334 may include a predictive output generation engine 336 for generating predictions or synthesized responses and an output validation engine 338 for verifying, filtering and validating generated outputs against predefined criteria and reference data. Further, the machine-learning system 300 may include a front end 340 configured to present validated outputs to end users and to collect interaction signals. Further, the machine-learning system 300 may include an outcome metrics module 342 configured to compute performance measures, accuracy statistics and other evaluation metrics derived from model outputs and user interactions. Further, the generative response engine 334 may be implemented on the computing device.

Further, the machine-learning system 300 may include a feedback engine 344 configured to aggregate outcome metrics and user feedback and to format such information for reuse. Further, the machine-learning system 300 may include a model refinement engine 346 configured to receive feedback from the feedback engine 344 and the outcome metrics module 342, and to effect iterative updates to the ML modeling engine 324 and to the ML algorithms database 332. Further, the components are communicatively coupled so that data and control signals are exchanged among the repositories (304, 306, 308), the data input engine 310, the featurization engine 316, the ML modeling engine 324 (with algorithmic support from the ML algorithms database 332), the generative response engine 334 and the front end 340 for output generation. Further, the outcome metrics 342 and the feedback engine 344 provide closed-loop signals to the model refinement engine 346 to enable retraining, parameter adjustment and algorithm selection, thereby enabling cooperative execution of data acquisition, feature engineering, model construction, output generation, validation, evaluation and iterative refinement within the disclosed machine-learning system 300. Further, the feedback engine 344 may be implemented on the computing device.

Any or each engine of the machine-learning system 300 may be and/or may include a module (e.g., a program module), which may be a hardware unit configured to be used with other components or a part of a program that performs a particular function. Further, any or each engine of the machine-learning system 300 may be implemented using a computing device.

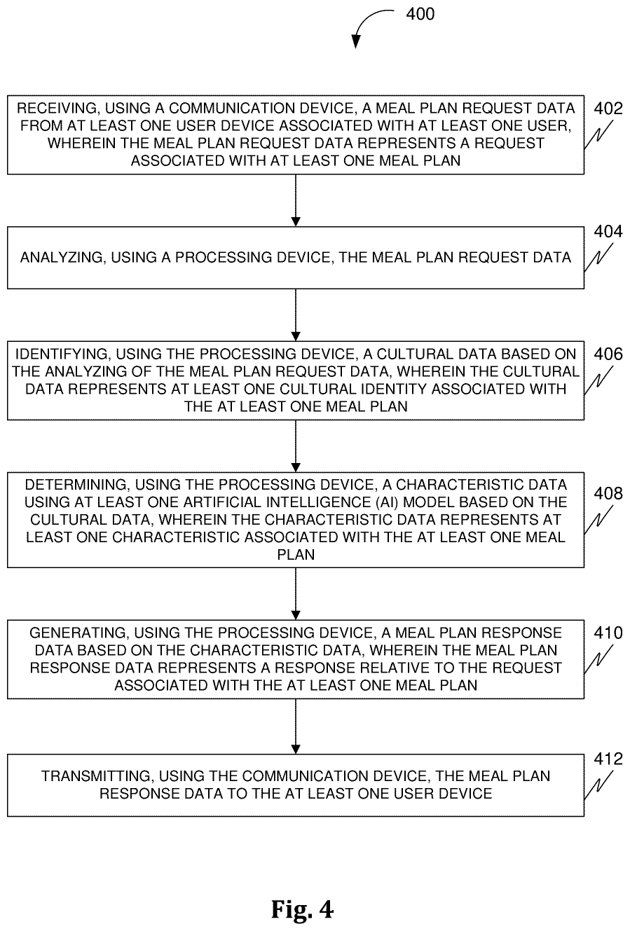

FIG. 4 illustrates a flowchart of a method 400 for providing a meal plan suggestion to a user, in accordance with some embodiments. Accordingly, the method 400 may include a step 402 of receiving, using a communication device 1102, a meal plan request data from one or more user devices associated with one or more users. Further, the meal plan request data represents a request associated with one or more meal plans. Further, the method 400 may include a step 404 of analyzing, using a processing device 1104, the meal plan request data. Further, the method 400 may include a step 406 of identifying, using the processing device 1104, a cultural data based on the analyzing of the meal plan request data. Further, the cultural data represents one or more cultural identities associated with the one or more meal plans. Further, the method 400 may include a step 408 of determining, using the processing device 1104, a characteristic data using one or more artificial intelligence (AI) models based on the cultural data. Further, the characteristic data represents one or more characteristics associated with the one or more meal plans. Further, the method 400 may include a step 410 of generating, using the processing device 1104, a meal plan response data based on the characteristic data. Further, the meal plan response data represents a response relative to the request associated with the one or more meal plans. Further, the method 400 may include a step 412 of transmitting, using the communication device 1102, the meal plan response data to the one or more user devices.

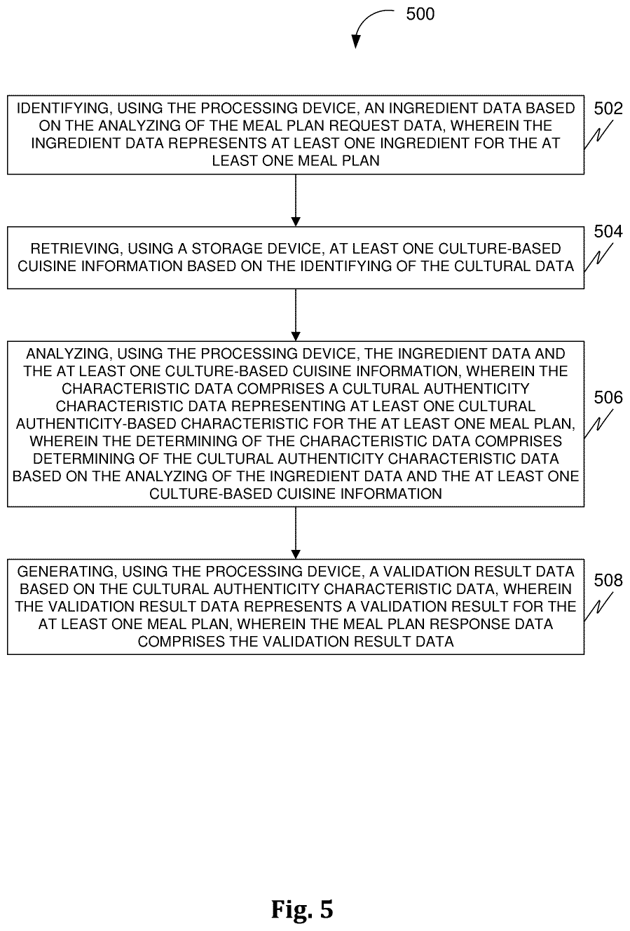

FIG. 5 illustrates a flowchart of a method 500 for providing a meal plan suggestion to a user including generating, using the processing device 1104, a validation result data, in accordance with some embodiments. Further, in some embodiments, the meal plan request data may include a meal plan validation data representing a request for a validation of the one or more meal plans. Further, the method 500 may include a step 502 of identifying, using the processing device 1104, an ingredient data based on the analyzing of the meal plan request data. Further, the ingredient data represents one or more ingredients for the one or more meal plans. Further, the method 500 may include a step 504 of retrieving, using a storage device 1106, one or more culture-based cuisine information based on the identifying of the cultural data. Further, the method 500 may include a step 506 of analyzing, using the processing device 1104, the ingredient data and the one or more culture-based cuisine information. Further, the characteristic data includes a cultural authenticity characteristic data representing one or more cultural authenticity-based characteristics for the one or more meal plans. Further, the determining of the characteristic data includes determining of the cultural authenticity characteristic data based on the analyzing of the ingredient data and the one or more culture-based cuisine information. Further, the method 500 may include a step 508 of generating, using the processing device 1104, a validation result data based on the cultural authenticity characteristic data. Further, the validation result data represents a validation result for the one or more meal plans. Further, the meal plan response data includes the validation result data.

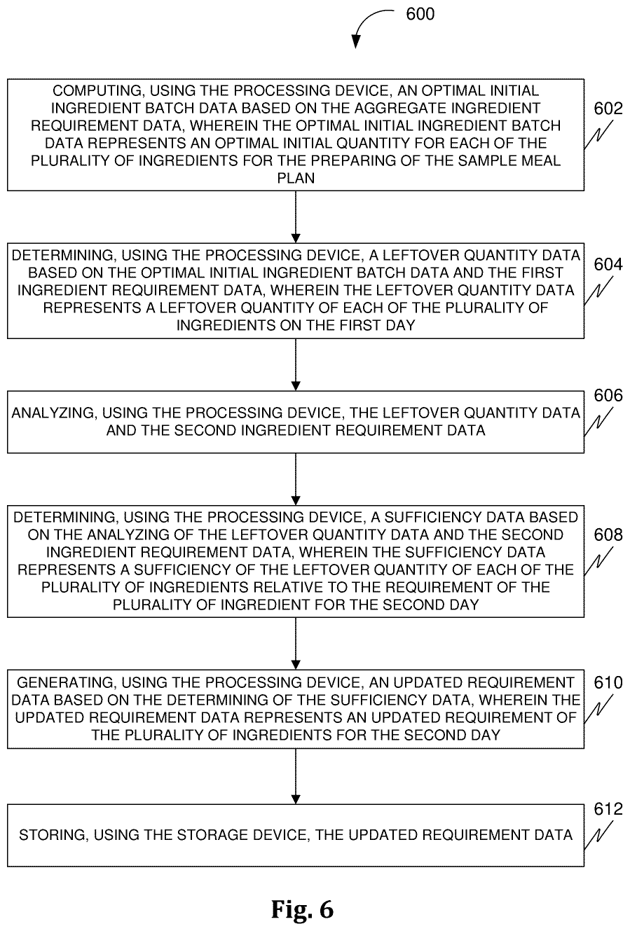

FIG. 6 illustrates a flowchart of a method 600 for providing a meal plan suggestion to a user including generating, using the processing device 1104, an updated requirement data, in accordance with some embodiments. Further, in some embodiments, the meal plan validation data may include a sample meal plan data representing a sample meal plan associated with two or more days. Further, the identifying of the ingredient data for the one or more meal plans may include identifying an aggregate ingredient requirement data representing an aggregate requirement of two or more ingredients for preparing the sample meal plan for the two or more days. Further, the two or more days may include a first day and a second day. Further, the second day occurs later than the first day. Further, the aggregate ingredient requirement data may include a first ingredient requirement data corresponding to a requirement of the two or more ingredients for the first day and a second ingredient requirement data corresponding to a requirement of the two or more ingredients for the second day. Further, the method 600 may include a step 602 of computing, using the processing device 1104, an optimal initial ingredient batch data based on the aggregate ingredient requirement data. Further, the optimal initial ingredient batch data represents an optimal initial quantity for each of the two or more ingredients for the preparing of the sample meal plan. Further, the identifying of the ingredient data for the one or more meal plans may include identifying an aggregate ingredient requirement data representing an aggregate requirement of two or more ingredients for preparing the sample meal plan for the two or more days. Further, the second day occurs later than the first day. Further, the method 600 may include a step 604 of determining, using the processing device 1104, a leftover quantity data based on the optimal initial ingredient batch data and the first ingredient requirement data. Further, the leftover quantity data represents a leftover quantity of each of the two or more ingredients on the first day. Further, the identifying of the ingredient data for the one or more meal plans may include identifying an aggregate ingredient requirement data representing an aggregate requirement of two or more ingredients for preparing the sample meal plan for the two or more days. Further, the second day occurs later than the first day. Further, the method 600 may include a step 606 of analyzing, using the processing device 1104, the leftover quantity data and the second ingredient requirement data. Further, the identifying of the ingredient data for the one or more meal plans may include identifying an aggregate ingredient requirement data representing an aggregate requirement of two or more ingredients for preparing the sample meal plan for the two or more days. Further, the second day occurs later than the first day. Further, the method 600 may include a step 608 of determining, using the processing device 1104, a sufficiency data based on the analyzing of the leftover quantity data and the second ingredient requirement data. Further, the sufficiency data represents a sufficiency of the leftover quantity of each of the two or more ingredients relative to the requirement of the two or more ingredients for the second day. Further, the identifying of the ingredient data for the one or more meal plans may include identifying an aggregate ingredient requirement data representing an aggregate requirement of two or more ingredients for preparing the sample meal plan for the two or more days. Further, the second day occurs later than the first day. Further, the method 600 may include a step 610 of generating, using the processing device 1104, an updated requirement data based on the determining of the sufficiency data. Further, the updated requirement data represents an updated requirement of the two or more ingredients for the second day. Further, the identifying of the ingredient data for the one or more meal plans may include identifying an aggregate ingredient requirement data representing an aggregate requirement of two or more ingredients for preparing the sample meal plan for the two or more days. Further, the second day occurs later than the first day. Further, the method 600 may include a step 612 of storing, using the storage device 1106, the updated requirement data.

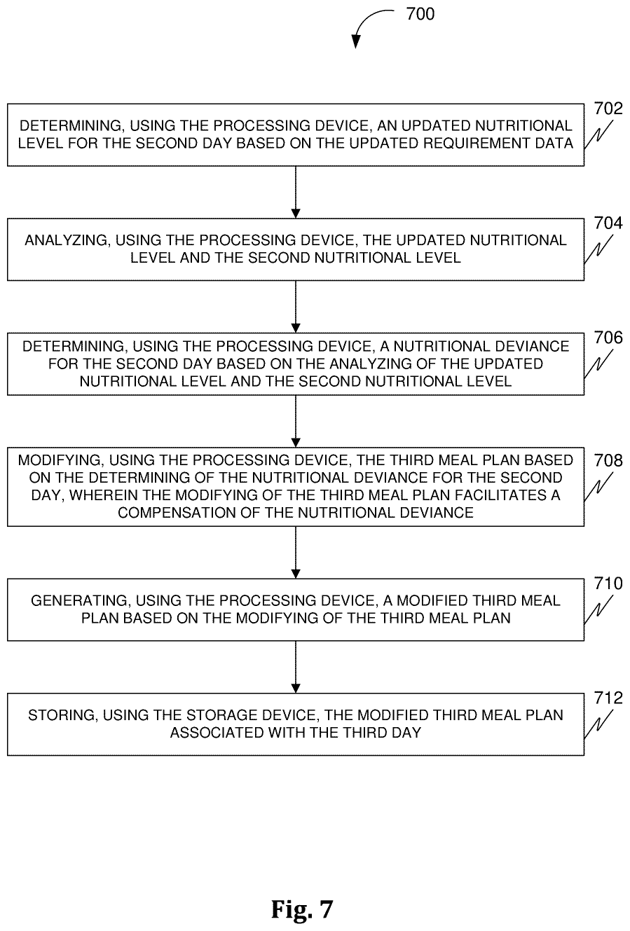

FIG. 7 illustrates a flowchart of a method 700 for providing a meal plan suggestion to a user including generating, using the processing device 1104, a modified third meal plan, in accordance with some embodiments. Further, in some embodiments, the sample meal plan may include a first meal plan associated with the first day, a second meal plan associated with the second day, and a third meal plan associated with a third day. Further, the third day occurs later than the second day. Further, the sample meal plan may be associated with a nutritional level. Further, the first meal plan may be associated with a first nutritional level, the second meal plan may be associated with a second nutritional level, and the third meal plan may be associated with a third nutritional level. Further, the method 700 may include a step 702 of determining, using the processing device 1104, an updated nutritional level for the second day based on the updated requirement data. Further, the sample meal plan may include a first meal plan associated with the first day, a second meal plan associated with the second day, and a third meal plan associated with a third day. Further, the sample meal plan may be associated with a nutritional level. Further, the method 700 may include a step 704 of analyzing, using the processing device 1104, the updated nutritional level and the second nutritional level. Further, the sample meal plan may include a first meal plan associated with the first day, a second meal plan associated with the second day, and a third meal plan associated with a third day. Further, the sample meal plan may be associated with a nutritional level. Further, the method 700 may include a step 706 of determining, using the processing device 1104, a nutritional deviance for the second day based on the analyzing of the updated nutritional level and the second nutritional level. Further, the sample meal plan may include a first meal plan associated with the first day, a second meal plan associated with the second day, and a third meal plan associated with a third day. Further, the sample meal plan may be associated with a nutritional level. Further, the method 700 may include a step 708 of modifying, using the processing device 1104, the third meal plan based on the determining of the nutritional deviance for the second day. Further, the modifying of the third meal plan facilitates a compensation of the nutritional deviance. Further, the sample meal plan may include a first meal plan associated with the first day, a second meal plan associated with the second day, and a third meal plan associated with a third day. Further, the sample meal plan may be associated with a nutritional level. Further, the method 700 may include a step 710 of generating, using the processing device 1104, a modified third meal plan based on the modifying of the third meal plan. Further, the sample meal plan may include a first meal plan associated with the first day, a second meal plan associated with the second day, and a third meal plan associated with a third day. Further, the sample meal plan may be associated with a nutritional level. Further, the method 700 may include a step 712 of storing, using the storage device 1106, the modified third meal plan associated with the third day.

In some embodiments, the one or more culture-based cuisine information includes a mandatory ingredient data representing one or more mandatory ingredients included for ensuring a cultural authenticity, a prohibited ingredient data representing one or more prohibited ingredients excluded for ensuring the cultural authenticity, a characteristic spice data representing one or more characteristic spices contributing to the cultural authenticity, and an authenticity threshold data representing one or more authenticity threshold values for the one or more region relatives to the cultural authenticity.

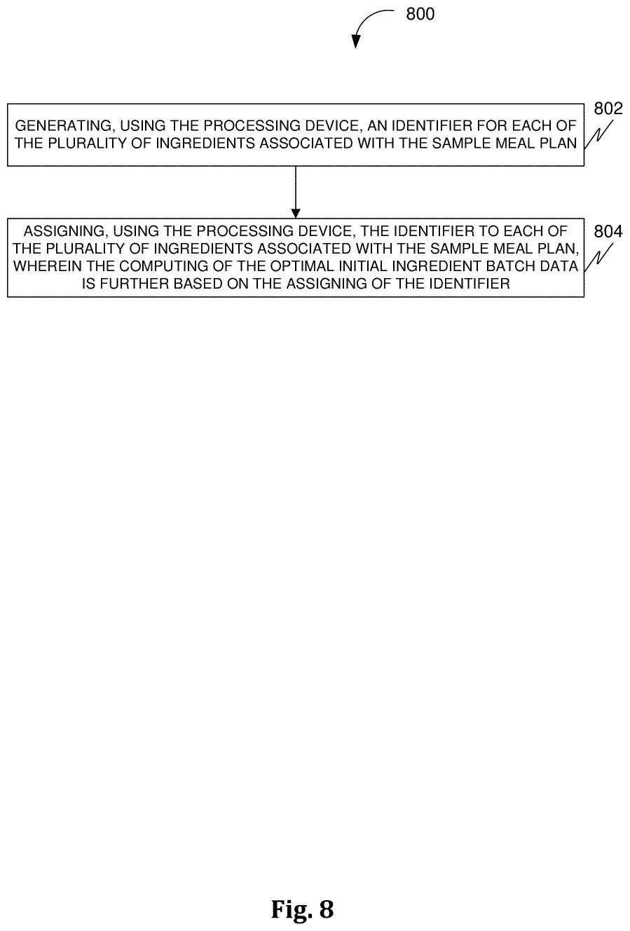

FIG. 8 illustrates a flowchart of a method 800 for providing a meal plan suggestion to a user including assigning, using the processing device 1104, the identifier to each of the plurality of ingredients associated with the sample meal plan, in accordance with some embodiments. Further, in some embodiments, the method 800 may include a step 802 of generating, using the processing device 1104, an identifier for each of the two or more ingredients associated with the sample meal plan. Further, in some embodiments, the method 800 may include a step 804 of assigning, using the processing device 1104, the identifier to each of the two or more ingredients associated with the sample meal plan. Further, the computing of the optimal initial ingredient batch data may be further based on the assigning of the identifier.

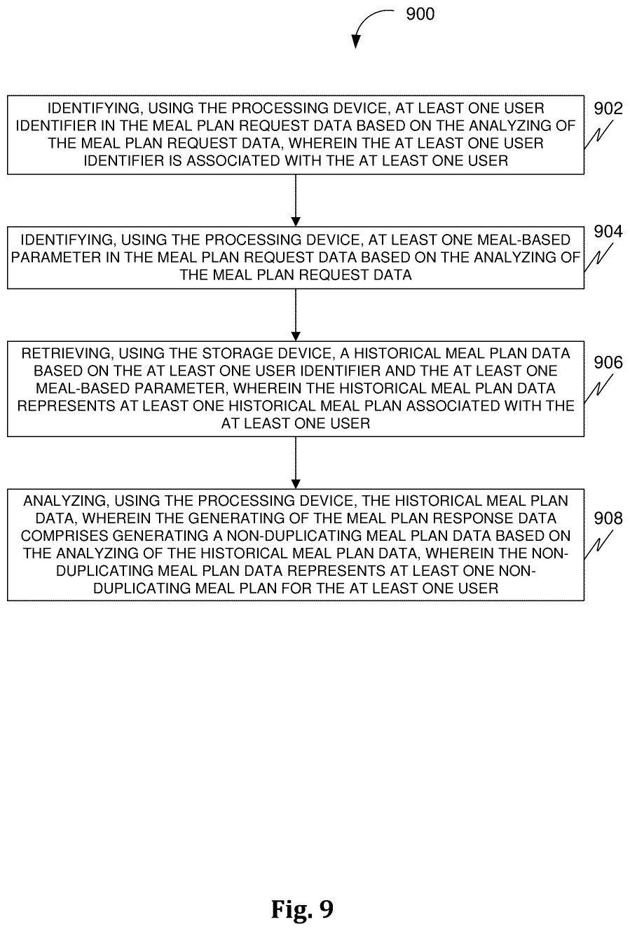

FIG. 9 illustrates a flowchart of a method 900 for providing a meal plan suggestion to a user including analyzing, using the processing device 1104, the historical meal plan data, in accordance with some embodiments. Further, in some embodiments, the method 900 may include a step 902 of identifying, using the processing device 1104, one or more user identifiers in the meal plan request data based on the analyzing of the meal plan request data. Further, the one or more user identifiers may be associated with the one or more users. Further, in some embodiments, the method 900 may include a step 904 of identifying, using the processing device 1104, one or more meal-based parameters in the meal plan request data based on the analyzing of the meal plan request data. Further, in some embodiments, the method 900 may include a step 906 of retrieving, using the storage device 1106, a historical meal plan data based on the one or more user identifiers and the one or more meal-based parameters. Further, the historical meal plan data represents one or more historical meal plans associated with the one or more users. Further, in some embodiments, the method 900 may include a step 908 of analyzing, using the processing device 1104, the historical meal plan data. Further, the generating of the meal plan response data includes generating a non-duplicating meal plan data based on the analyzing of the historical meal plan data. Further, the non-duplicating meal plan may data represents one or more non-duplicating meal plans for the one or more users.

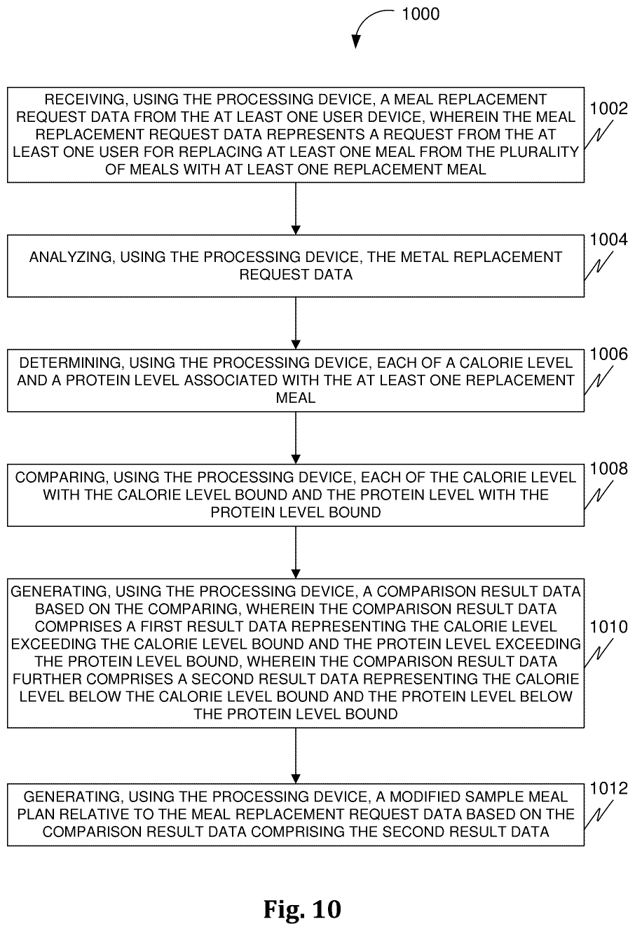

FIG. 10 illustrates a flowchart of a method 1000 for providing a meal plan suggestion to a user including generating, using the processing device 1104, a modified sample meal plan relative to the meal replacement request data, in accordance with some embodiments. Further, in some embodiments, the sample meal plan associated with the two or more days may include two or more meals. Further, each of the two or more meals may be associated with one or more of a calorie level bound and a protein level bound. Further, the method 1000 may include a step 1002 of receiving, using the processing device 1104, a meal replacement request data from the one or more user devices. Further, the meal replacement request data represents a request from the one or more users for replacing one or more meals from the two or more meals with one or more replacement meals. Further, the sample meal plan associated with the two or more days may include two or more meals. Further, the method 1000 may include a step 1004 of analyzing, using the processing device 1104, the metal replacement request data. Further, the sample meal plan associated with the two or more days may include two or more meals. Further, the method 1000 may include a step 1006 of determining, using the processing device 1104, each of a calorie level and a protein level associated with the one or more replacement meals. Further, the sample meal plan associated with the two or more days may include two or more meals. Further, the method 1000 may include a step 1008 of comparing, using the processing device 1104, each of the calorie level with the calorie level bound and the protein level with the protein level bound. Further, the sample meal plan associated with the two or more days may include two or more meals. Further, the method 1000 may include a step 1010 of generating, using the processing device 1104, a comparison result data based on the comparing. Further, the comparison result data includes a first result data representing the calorie level exceeding the calorie level bound and the protein level exceeding the protein level bound. Further, the comparison result data further includes a second result data representing the calorie level below the calorie level bound and the protein level below the protein level bound. Further, the sample meal plan associated with the two or more days may include two or more meals. Further, the method 1000 may include a step 1012 of generating, using the processing device 1104, a modified sample meal plan relative to the meal replacement request data based on the comparison result data comprising the second result data.

In some embodiments, the cultural data includes a geographic-regional group data representing a group associated with one or more geographical regions and religious sub-group data representing one or more religious sub-groups within the one or more geographical regions.

In some embodiments, the validation result includes at least one a validation indication and an invalidation indication for the one or more meal plans. Further, the generating of the meal plan response data includes generating of a meal regeneration suggestion data based on the validation result includes the invalidation indication. Further, the meal regeneration suggestion data represents one or more suggestions for regeneration of the one or more meal plans.

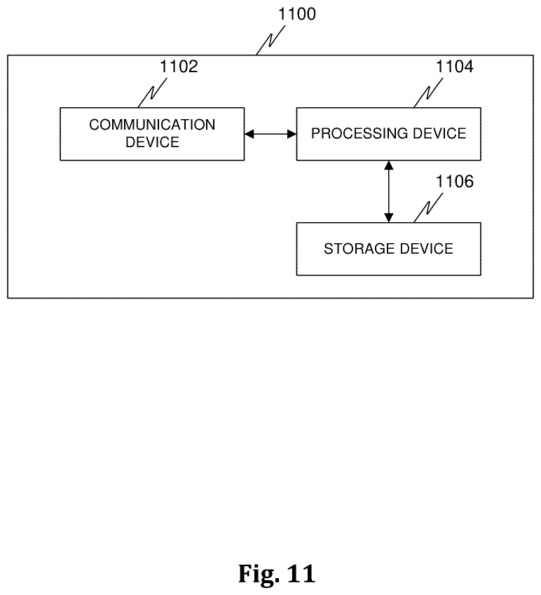

FIG. 11 illustrates a block diagram of a system 1100 for providing a meal plan suggestion to a user, in accordance with some embodiments. Accordingly, the system 1100 may include a communication device 1102. Further, the communication device 1102 may be configured for receiving a meal plan request data from one or more user devices associated with one or more users. Further, the meal plan request data represents a request associated with one or more meal plans. Further, the communication device 1102 may be configured for transmitting a meal plan response data to the one or more user devices. Further, the system 1100 may include a processing device 1104 communicatively coupled with the communication device 1102. Further, the processing device 1104 may be configured for analyzing the meal plan request data. Further, the processing device 1104 may be configured for identifying a cultural data based on the analyzing of the meal plan request data. Further, the cultural data represents one or more cultural identities associated with the one or more meal plans. Further, the processing device 1104 may be configured for determining a characteristic data using one or more artificial intelligence (AI) models based on the cultural data. Further, the characteristic data represents one or more characteristics associated with the one or more meal plans. Further, the processing device 1104 may be configured for generating the meal plan response data based on the characteristic data. Further, the meal plan response data represents a response relative to the request associated with the one or more meal plans.

Further, in some embodiments, the meal plan request data may include a meal plan validation data representing a request for a validation of the one or more meal plans. Further, the processing device 1104 may be further configured for identifying an ingredient data based on the analyzing of the meal plan request data. Further, the ingredient data represents one or more ingredients for the one or more meal plans. Further, the processing device 1104 may be further configured for analyzing the ingredient data and one or more culture-based cuisine information. Further, the characteristic data includes a cultural authenticity characteristic data representing one or more cultural authenticity-based characteristics for the one or more meal plans. Further, the determining of the characteristic data includes determining of the cultural authenticity characteristic data based on the analyzing of the ingredient data and the one or more culture-based cuisine information. Further, the processing device 1104 may be further configured for generating a validation result data based on the cultural authenticity characteristic data. Further, the validation result data represents a validation result for the one or more meal plans. Further, the meal plan response data includes the validation result data. Further, the system 1100 further includes a storage device 1106 communicatively coupled with the processing device 1104. Further, the storage device 1106 may be configured for retrieving the one or more culture-based cuisine information based on the identifying of the cultural data.

Further, in some embodiments, the meal plan validation data may include a sample meal plan data representing a sample meal plan associated with two or more days. Further, the identifying of the ingredient data for the one or more meal plans may include identifying an aggregate ingredient requirement data representing an aggregate requirement of two or more ingredients for preparing the sample meal plan for the two or more days. Further, the two or more days may include a first day and a second day. Further, the second day occurs later than the first day. Further, the aggregate ingredient requirement data may include a first ingredient requirement data corresponding to a requirement of the two or more ingredients for the first day and a second ingredient requirement data corresponding to a requirement of the two or more ingredients for the second day. Further, the processing device 1104 may be further configured for computing an optimal initial ingredient batch data based on the aggregate ingredient requirement data. Further, the optimal initial ingredient batch data represents an optimal initial quantity for each of the two or more ingredients for the preparing of the sample meal plan. Further, the identifying of the ingredient data for the one or more meal plans may include identifying an aggregate ingredient requirement data representing an aggregate requirement of two or more ingredients for preparing the sample meal plan for the two or more days. Further, the second day occurs later than the first day. Further, the processing device 1104 may be further configured for determining a leftover quantity data based on the optimal initial ingredient batch data and the first ingredient requirement data. Further, the leftover quantity data represents a leftover quantity of each of the two or more ingredients on the first day. Further, the identifying of the ingredient data for the one or more meal plans may include identifying an aggregate ingredient requirement data representing an aggregate requirement of two or more ingredients for preparing the sample meal plan for the two or more days. Further, the second day occurs later than the first day. Further, the processing device 1104 may be further configured for analyzing the leftover quantity data and the second ingredient requirement data. Further, the identifying of the ingredient data for the one or more meal plans may include identifying an aggregate ingredient requirement data representing an aggregate requirement of two or more ingredients for preparing the sample meal plan for the two or more days. Further, the second day occurs later than the first day. Further, the processing device 1104 may be further configured for determining a sufficiency data based on the analyzing of the leftover quantity data and the second ingredient requirement data. Further, the sufficiency data represents a sufficiency of the leftover quantity of each of the two or more ingredients relative to the requirement of the two or more ingredients for the second day. Further, the identifying of the ingredient data for the one or more meal plans may include identifying an aggregate ingredient requirement data representing an aggregate requirement of two or more ingredients for preparing the sample meal plan for the two or more days. Further, the second day occurs later than the first day. Further, the processing device 1104 may be further configured for generating an updated requirement data based on the determining of the sufficiency data. Further, the updated requirement data represents an updated requirement of the two or more ingredients for the second day. Further, the storage device 1106 may be further configured for storing the updated requirement data.

Further, in some embodiments, the sample meal plan may include a first meal plan associated with the first day, a second meal plan associated with the second day, and a third meal plan associated with a third day. Further, the third day occurs later than the second day. Further, the sample meal plan may be associated with a nutritional level. Further, the first meal plan may be associated with a first nutritional level, the second meal plan may be associated with a second nutritional level, and the third meal plan may be associated with a third nutritional level. Further, the processing device 1104 may be further configured for determining an updated nutritional level for the second day based on the updated requirement data. Further, the sample meal plan may include a first meal plan associated with the first day, a second meal plan associated with the second day, and a third meal plan associated with a third day. Further, the sample meal plan may be associated with a nutritional level. Further, the processing device 1104 may be further configured for analyzing the updated nutritional level and the second nutritional level. Further, the sample meal plan may include a first meal plan associated with the first day, a second meal plan associated with the second day, and a third meal plan associated with a third day. Further, the sample meal plan may be associated with a nutritional level. Further, the processing device 1104 may be further configured for determining a nutritional deviance for the second day based on the analyzing of the updated nutritional level and the second nutritional level. Further, the sample meal plan may include a first meal plan associated with the first day, a second meal plan associated with the second day, and a third meal plan associated with a third day. Further, the sample meal plan may be associated with a nutritional level. Further, the processing device 1104 may be further configured for modifying the third meal plan based on the determining of the nutritional deviance for the second day. Further, the modifying of the third meal plan facilitates a compensation of the nutritional deviance. Further, the sample meal plan may include a first meal plan associated with the first day, a second meal plan associated with the second day, and a third meal plan associated with a third day. Further, the sample meal plan may be associated with a nutritional level. Further, the processing device 1104 may be further configured for generating a modified third meal plan based on the modifying of the third meal plan. Further, the storage device 1106 may be further configured for storing the modified third meal plan associated with the third day.

In some embodiments, the one or more culture-based cuisine information includes a mandatory ingredient data representing one or more mandatory ingredients included for ensuring a cultural authenticity, a prohibited ingredient data representing one or more prohibited ingredients excluded for ensuring the cultural authenticity, a characteristic spice data representing one or more characteristic spices contributing to the cultural authenticity, and an authenticity threshold data representing one or more authenticity threshold values for the one or more region relatives to the cultural authenticity.

Further, in some embodiments, the processing device 1104 may be further configured for generating an identifier for each of the two or more ingredients associated with the sample meal plan. Further, the processing device 1104 may be further configured for assigning the identifier to each of the two or more ingredients associated with the sample meal plan. Further, the computing of the optimal initial ingredient batch data may be further based on the assigning of the identifier.

Further, in some embodiments, the processing device 1104 may be further configured for identifying one or more user identifiers in the meal plan request data based on the analyzing of the meal plan request data. Further, the one or more user identifiers may be associated with the one or more users. Further, the processing device 1104 may be further configured for identifying one or more meal-based parameters in the meal plan request data based on the analyzing of the meal plan request data. Further, the processing device 1104 may be further configured for analyzing a historical meal plan data. Further, the generating of the meal plan response data includes generating a non-duplicating meal plan data based on the analyzing of the historical meal plan data. Further, the non-duplicating meal plan may data represents one or more non-duplicating meal plans for the one or more users. Further, the storage device 1106 may be further configured for retrieving the historical meal plan data based on the one or more user identifiers and the one or more meal-based parameters. Further, the historical meal plan may data represents one or more historical meal plans associated with the one or more users.

Further, in some embodiments, the sample meal plan associated with the two or more days may include two or more meals. Further, each of the two or more meals may be associated with one or more of a calorie level bound and a protein level bound. Further, the communication device 1102 may be further configured for receiving a meal replacement request data from the one or more user devices. Further, the meal replacement request data represents a request from the one or more users for replacing one or more meals from the two or more meals with one or more replacement meals. Further, the processing device 1104 may be further configured for analyzing the metal replacement request data. Further, the sample meal plan associated with the two or more days may include two or more meals. Further, the communication device 1102 may be further configured for receiving a meal replacement request data from the one or more user devices. Further, the processing device 1104 may be further configured for determining each of a calorie level and a protein level associated with the one or more replacement meals. Further, the sample meal plan associated with the two or more days may include two or more meals. Further, the communication device 1102 may be further configured for receiving a meal replacement request data from the one or more user devices. Further, the processing device 1104 may be further configured for comparing each of the calorie level with the calorie level bound and the protein level with the protein level bound. Further, the sample meal plan associated with the two or more days may include two or more meals. Further, the communication device 1102 may be further configured for receiving a meal replacement request data from the one or more user devices. Further, the processing device 1104 may be further configured for generating a comparison result data based on the comparing. Further, the comparison result data includes a first result data representing the calorie level exceeding the calorie level bound and the protein level exceeding the protein level bound. Further, the comparison result data further includes a second result data representing the calorie level below the calorie level bound and the protein level below the protein level bound. Further, the sample meal plan associated with the two or more days may include two or more meals. Further, the communication device 1102 may be further configured for receiving a meal replacement request data from the one or more user devices. Further, the processing device 1104 may be further configured for generating a modified sample meal plan relative to the meal replacement request data based on the comparison result data comprising the second result data.

In some embodiments, the cultural data includes a geographic-regional group data representing a group associated with one or more geographical regions and religious sub-group data representing one or more religious sub-groups within the one or more geographical regions.

In some embodiments, the validation result includes at least one a validation indication and an invalidation indication for the one or more meal plans. Further, the generating of the meal plan response data includes generating of a meal regeneration suggestion data based on the validation result includes the invalidation indication. Further, the meal regeneration suggestion data represents one or more suggestions for regeneration of the one or more meal plans.

In some embodiments, the one or more geographical regions include one or more of Kerala, Tamil Nadu, and Punjab. Further, the one or more religious sub-groups include one or more of Palakkad Iyer Brahmin and Jain.

In some embodiments, the one or more meal-based parameters include a duration associated with the one or more meal plans.

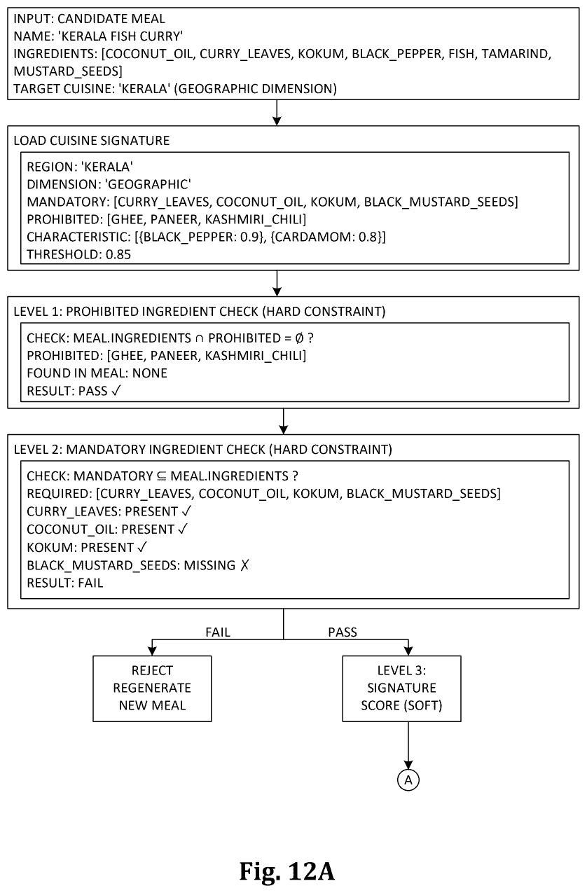

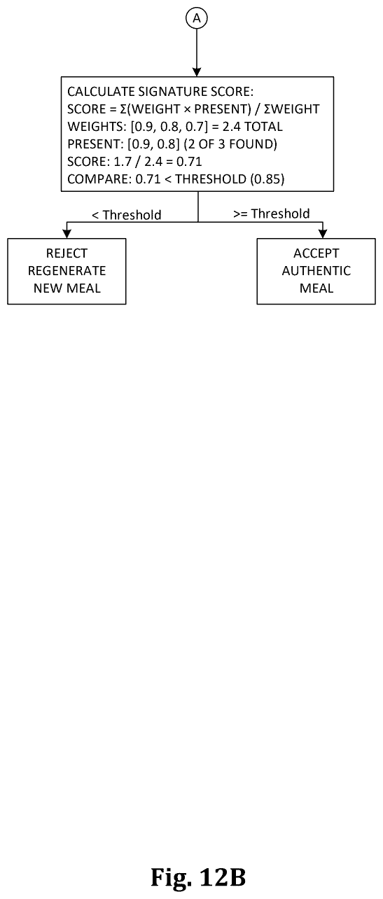

FIG. 12A and FIG. 12B illustrate a tri-level validation process for sub-regional cuisine fingerprinting, in accordance with some embodiments. Further, the system loads the appropriate cuisine signature based on the user's specified cultural identity (which may include geographic and/or religious dimensions). Further, the system applies two hard constraints (prohibited ingredients, mandatory ingredients) followed by a soft constraint (signature score). Further, meals failing any constraint are rejected and regenerated. Further, the threshold is configurable based on the specificity level: lower for broad regional (0.65-0.75), medium for state-level (0.80-0.85), and higher for religious sub-groups (0.90+).

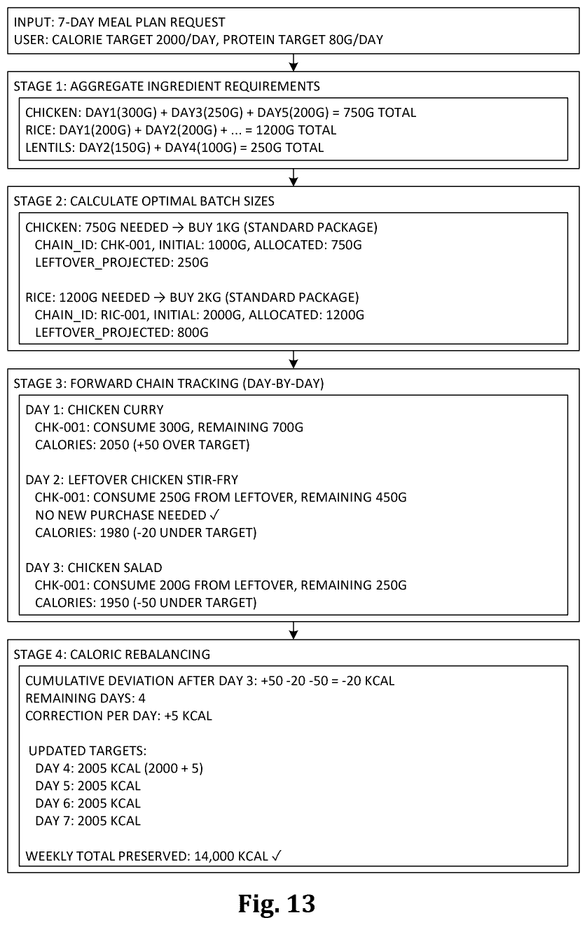

FIG. 13 illustrates a forward-chaining leftover optimization algorithm, in accordance with some embodiments. The system aggregates ingredient requirements across the planning period, calculates optimal batch sizes considering standard packaging, assigns chain identifiers for tracking, and dynamically rebalances nutritional targets when leftover consumption causes deviation. Further, the given approach significantly reduces ingredient waste compared to independent daily planning.

Further, FIG. 13 describes the following waste comparison of the disclosed system relative to the existing systems. Further, the traditional systems (independent daily): 1500 g purchased, 750 g used=50% waste. Further, using the forward-chaining procedure: 1000 g purchased, 750 g used=25% waste. Further, the improvement includes 50% reduction in ingredient waste.

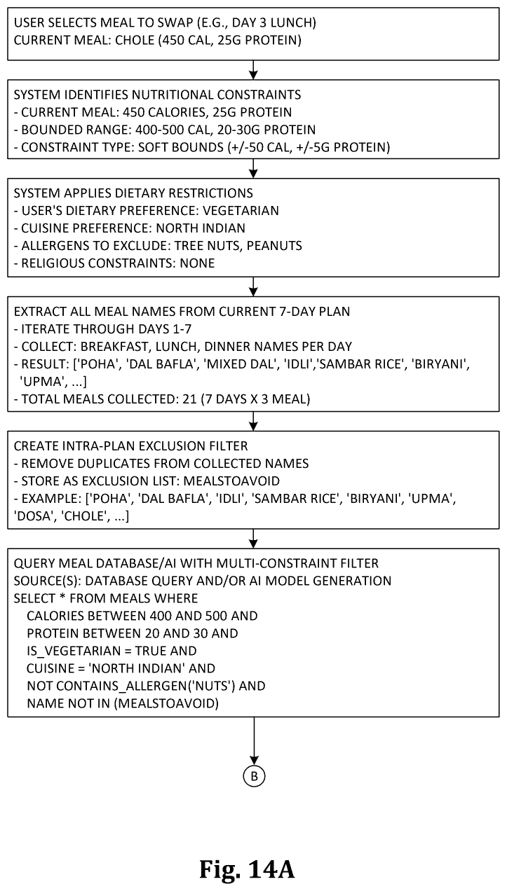

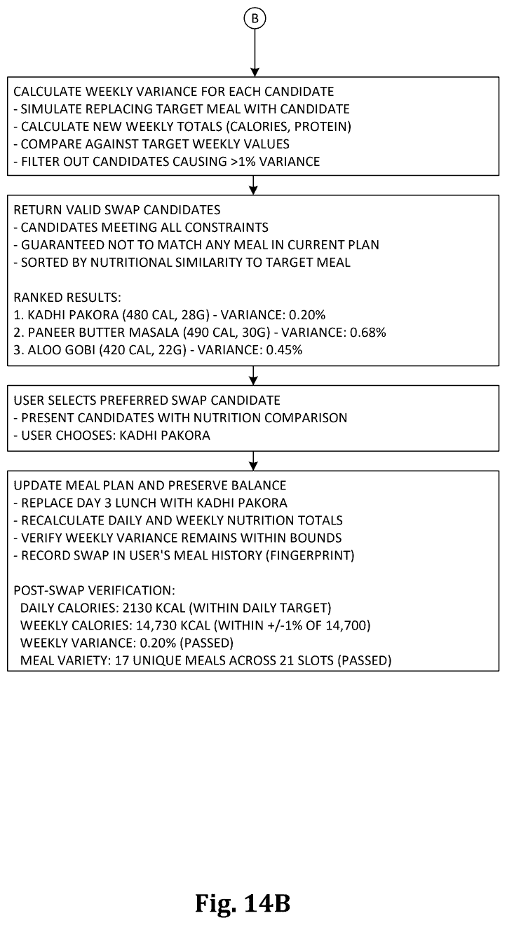

FIG. 14A and FIG. 14B illustrate a constraint-preserving bounded swap algorithm with intra-plan deduplication, in accordance with some embodiments. Further, steps 3.5-3.6 extract all meal names from the current 7-day plan to create an exclusion list, ensuring swap candidates do NOT match any existing meal in the active plan, maintaining variety across the full planning window. Further, the system applies multi-dimensional filtering (calories, protein, cuisine, dietary restrictions, intra-plan exclusion, weekly variance) to identify valid swap candidates, then ranks them by similarity score. Further, the given approach enables user customization while preserving nutritional and cultural goals without expert intervention.

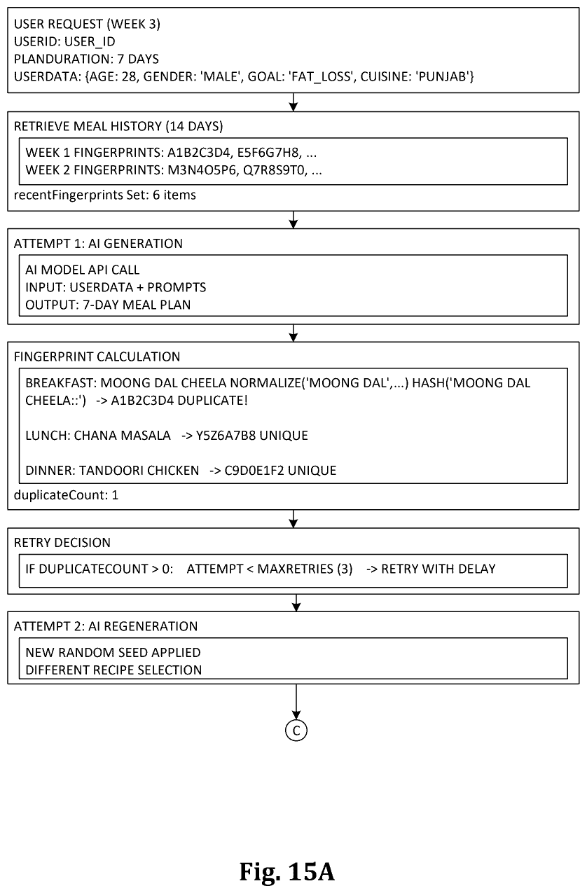

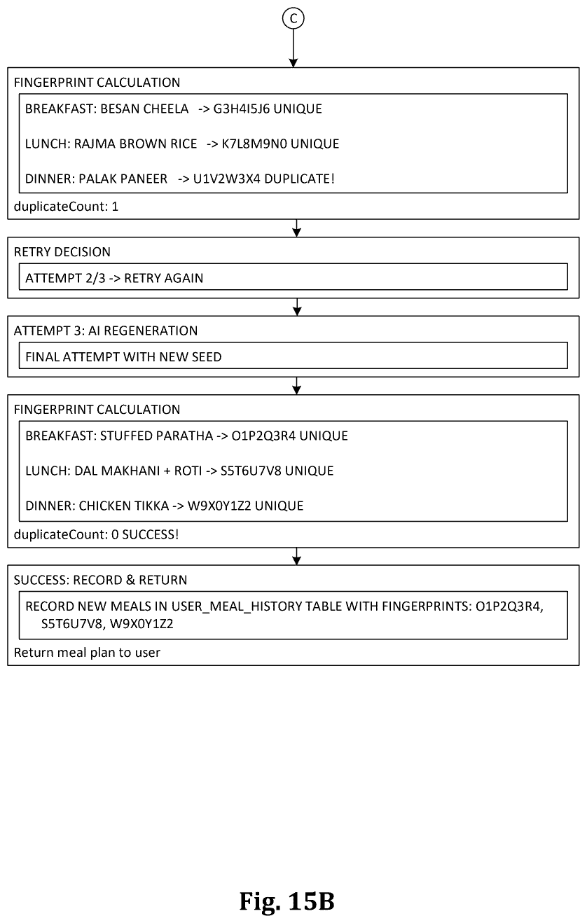

FIG. 15A and FIG. 15B illustrate a historical meal deduplication flow with content-based fingerprinting and retry logic, in accordance with some embodiments. Further, the system retrieves the user's meal history, generates candidate meals, calculates fingerprints, detects duplicates, and retries with different random seeds until a unique plan is generated or the configurable maximum retries are exhausted. Further, the given example shows a configuration with 3 maximum attempts.

Further, in accordance with each of the FIG. 15A and FIG. 15B, the ‘Unique’ label represents that the fingerprint is not present in history. Further, the ‘Duplicate!’ label represents that the fingerprint exists in history. Further, a1b2c3d4 code represents an 8-character hex fingerprint.

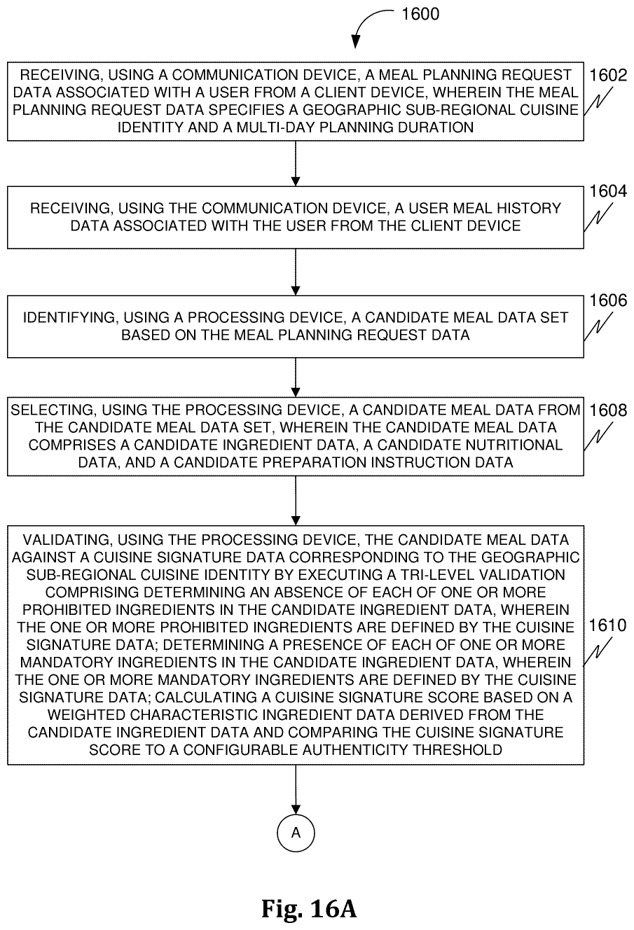

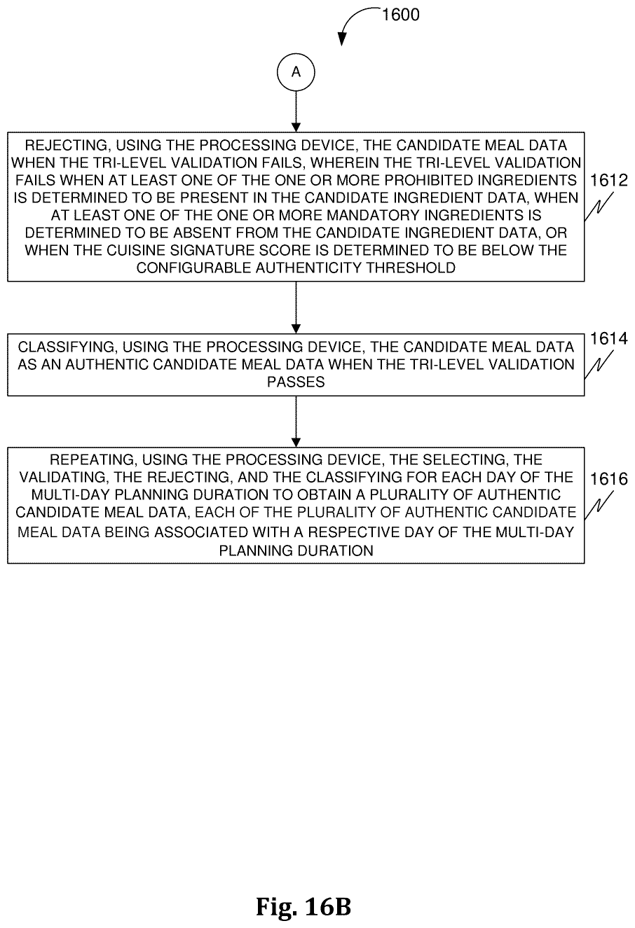

FIGS. 16A-16B illustrate a flowchart of a method 1600 of facilitating culturally-authentic and non-repetitive meal planning, in accordance with some embodiments. Accordingly, the method 1600 may include a step 1602 of receiving, using a communication device 1902, a meal planning request data associated with a user from a client device 1908. Further, the meal planning request data specifies a geographic sub-regional cuisine identity and a multi-day planning duration. Further, the method 1600 may include a step 1604 of receiving, using the communication device 1902, a user meal history data associated with the user from the client device. Further, the method 1600 may include a step 1606 of identifying, using a processing device 1904, a candidate meal data set based on the meal planning request data. Further, the method 1600 may include a step 1608 of selecting, using the processing device 1904, a candidate meal data from the candidate meal data set. Further, the candidate meal data may include a candidate ingredient data, a candidate nutritional data, and a candidate preparation instruction data. Further, the method 1600 may include a step 1610 of validating, using the processing device 1904, the candidate meal data against a cuisine signature data corresponding to the geographic sub-regional cuisine identity by executing a tri-level validation. Further, the tri-level validation may include determining an absence of each of one or more prohibited ingredients in the candidate ingredient data. Further, the one or more prohibited ingredients are defined by the cuisine signature data. Further, the tri-level validation may include determining a presence of each of one or more mandatory ingredients in the candidate ingredient data. Further, the one or more mandatory ingredients are defined by the cuisine signature data. Further, the tri-level validation may include calculating a cuisine signature score based on a weighted characteristic ingredient data derived from the candidate ingredient data and comparing the cuisine signature score to a configurable authenticity threshold. Further, the method 1600 may include a step 1612 of rejecting, using the processing device 1604, the candidate meal data when the tri-level validation fails. Further, the tri-level validation fails when at least one of the one or more prohibited ingredients is determined to be present in the candidate ingredient data, when at least one of the one or more mandatory ingredients is determined to be absent from the candidate ingredient data, or when the cuisine signature score is determined to be below the configurable authenticity threshold. Further, the method 1600 may include a step 1614 of classifying, using the processing device 1904, the candidate meal data as an authentic candidate meal data when the tri-level validation passes. Further, the method 1600 may include a step 1616 of repeating, using the processing device 1904, the selecting, the validating, the rejecting, and the classifying for each day of the multi-day planning duration to obtain a plurality of authentic candidate meal data, each of the plurality of authentic candidate meal data being associated with a respective day of the multi-day planning duration.

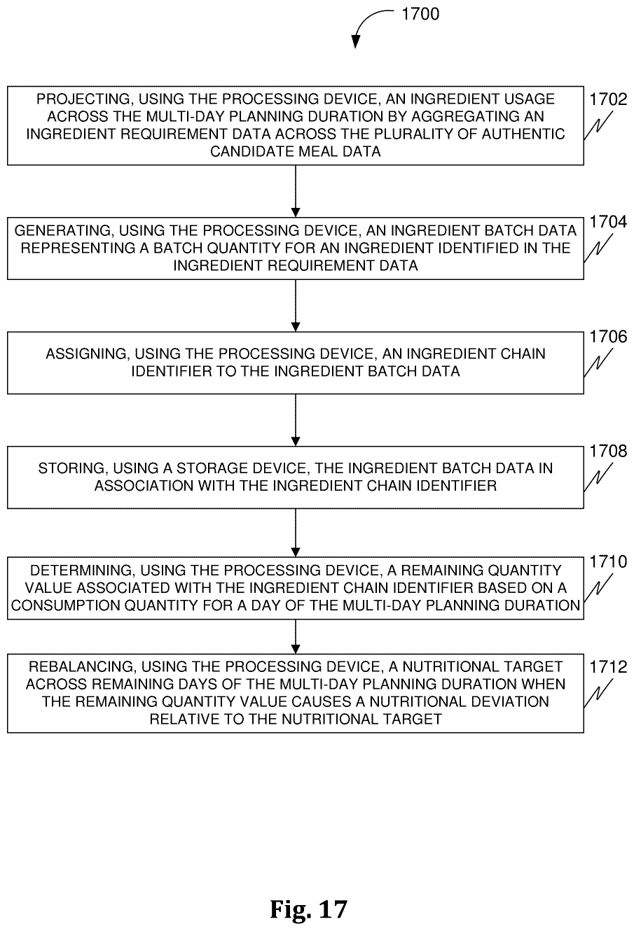

FIG. 17 illustrates a flowchart of a method 1700 of facilitating the culturally-authentic and non-repetitive meal planning, in accordance with some embodiments. Further, the method 1700 may include a step 1702 of projecting, using the processing device 1904, an ingredient usage across the multi-day planning duration by aggregating an ingredient requirement data across the plurality of authentic candidate meal data. Further, the method 1700 may include a step 1704 of generating, using the processing device 1904, an ingredient batch data representing a batch quantity for an ingredient identified in the ingredient requirement data. Further, the method 1700 may include a step 1706 of assigning, using the processing device 1904, an ingredient chain identifier to the ingredient batch data. Further, the method 1700 may include a step 1708 of storing, using a storage device 1906, the ingredient batch data in association with the ingredient chain identifier. Further, the method 1700 may include a step 1710 of determining, using the processing device 1904, a remaining quantity value associated with the ingredient chain identifier based on a consumption quantity for a day of the multi-day planning duration. Further, the method 1700 may include a step 1712 of rebalancing, using the processing device 1904, a nutritional target across remaining days of the multi-day planning duration when the remaining quantity value causes a nutritional deviation relative to the nutritional target.

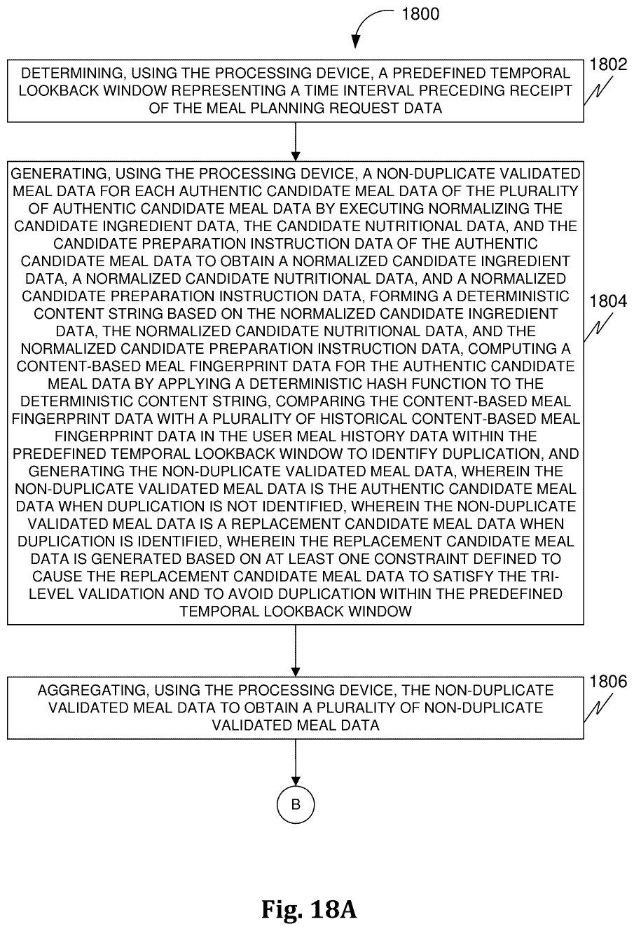

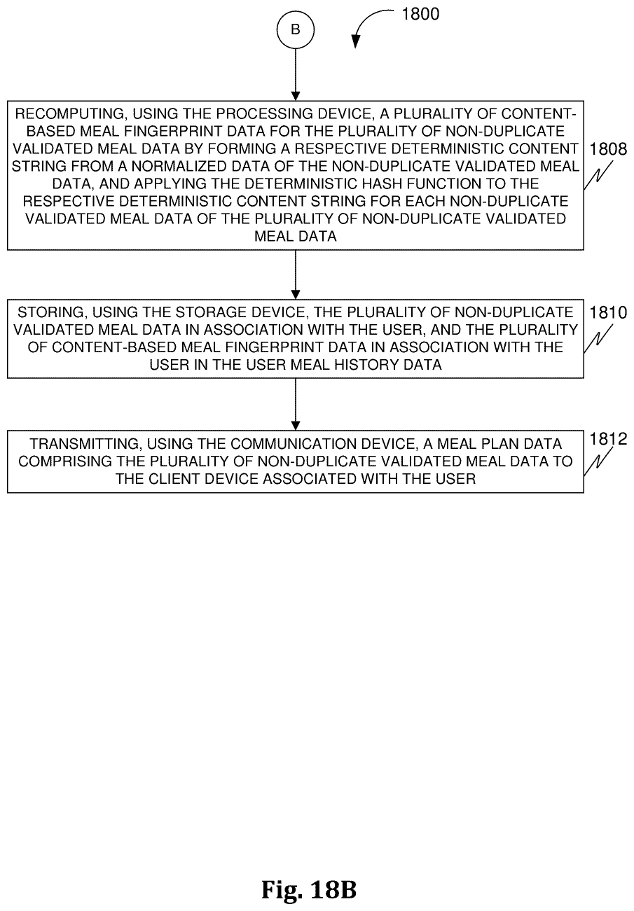

FIGS. 18A-18B illustrate a flowchart of a method 1800 of facilitating the culturally-authentic and non-repetitive meal planning, in accordance with some embodiments. Further, the method 1800 may include a step 1802 of determining, using the processing device 1904, a predefined temporal lookback window representing a time interval preceding receipt of the meal planning request data. Further, the method 1800 may include a step 1804 of generating, using the processing device 1904, a non-duplicate validated meal data for each authentic candidate meal data of the plurality of authentic candidate meal data by executing: normalizing the candidate ingredient data, the candidate nutritional data, and the candidate preparation instruction data of the authentic candidate meal data to obtain a normalized candidate ingredient data, a normalized candidate nutritional data, and a normalized candidate preparation instruction data; forming a deterministic content string based on the normalized candidate ingredient data, the normalized candidate nutritional data, and the normalized candidate preparation instruction data; computing a content-based meal fingerprint data for the authentic candidate meal data by applying a deterministic hash function to the deterministic content string; comparing the content-based meal fingerprint data with a plurality of historical content-based meal fingerprint data in the user meal history data within the predefined temporal lookback window to identify duplication; and generating the non-duplicate validated meal data. Further, the non-duplicate validated meal data is the authentic candidate meal data when duplication is not identified. Further, the non-duplicate validated meal data is a replacement candidate meal data when duplication is identified. Further, the replacement candidate meal data is generated based on at least one constraint defined to cause the replacement candidate meal data to satisfy the tri-level validation and to avoid duplication within the predefined temporal lookback window. Further, the method 1800 may include a step 1806 of aggregating, using the processing device 1904, the non-duplicate validated meal data to obtain a plurality of non-duplicate validated meal data. Further, the method 1800 may include a step 1808 of recomputing, using the processing device 1904, a plurality of content-based meal fingerprint data for the plurality of non-duplicate validated meal data by forming a respective deterministic content string from a normalized data of the non-duplicate validated meal data, and applying the deterministic hash function to the respective deterministic content string for each non-duplicate validated meal data of the plurality of non-duplicate validated meal data. Further, the method 1800 may include a step 1810 of storing, using the storage device 1906, the plurality of non-duplicate validated meal data in association with the user, and the plurality of content-based meal fingerprint data in association with the user in the user meal history data. Further, the method 1800 may include a step 1812 of transmitting, using the communication device 1902, a meal plan data comprising the plurality of non-duplicate validated meal data to the client device 1908 associated with the user.

In some embodiments, the calculating of the cuisine signature score may include normalizing the candidate ingredient data to obtain a canonical ingredient name data. Further, the calculating of the cuisine signature score may further include normalizing the candidate ingredient data to obtain a canonical ingredient quantity data. Further, the calculating of the cuisine signature score may further include constructing a canonical ingredient vector based on the canonical ingredient name data and the canonical ingredient quantity data. Further, the calculating of the cuisine signature score may further include weighting the canonical ingredient vector using a cuisine-specific weight parameter defined in the cuisine signature data to obtain a weighted canonical ingredient vector. Further, the calculating of the cuisine signature score may further include computing the cuisine signature score based on the weighted canonical ingredient vector.

In some embodiments, the comparing of the cuisine signature score to the configurable authenticity threshold may include selecting the configurable authenticity threshold based on a geographic specificity level associated with the geographic sub-regional cuisine identity. Further, the comparing of the cuisine signature score to the configurable authenticity threshold may further include determining a threshold satisfaction condition based on the cuisine signature score and the configurable authenticity threshold. Further, the comparing of the cuisine signature score to the configurable authenticity threshold may further include determining an authenticity result data representing whether the threshold satisfaction condition is satisfied or not.

In some embodiments, the projecting of the ingredient usage may include aggregating an ingredient quantity data across the multi-day planning duration. Further, the projecting of the ingredient usage may further include generating the ingredient requirement data based on the ingredient quantity data.

In some embodiments, the generating of the ingredient batch data may include determining a batch purchase quantity for the ingredient based on the ingredient requirement data. Further, the generating of the ingredient batch data may further include initializing the batch quantity in the ingredient batch data with an initial value based on the batch purchase quantity. Further, the generating of the ingredient batch data is further based on the initializing. Further, the ingredient batch data is generated prior to assigning the ingredient chain identifier.

In some embodiments, the determining of the remaining quantity value may include retrieving the ingredient batch data associated with the ingredient chain identifier. Further, the determining of the remaining quantity value may further include computing the remaining quantity value by subtracting the consumption quantity from the initial value of the batch quantity in the ingredient batch data.

In some embodiments, the rebalancing of the nutritional target may include calculating a cumulative nutritional deviation attributable to the remaining quantity value. Further, the rebalancing of the nutritional target may further include determining a number of the remaining days in the multi-day planning duration. Further, the rebalancing of the nutritional target may further include computing a per-day nutritional adjustment value based on the cumulative nutritional deviation and the number of the remaining days. Further, the rebalancing of the nutritional target may further include updating a daily nutritional target for each of the number of the remaining days using the per-day nutritional adjustment value. Further, the rebalancing of the nutritional target is further based on the updating of the daily nutritional target.

In some embodiments, the normalizing of the candidate ingredient data, the candidate nutritional data, and the candidate preparation instruction data may include converting an ingredient name in the candidate ingredient data to a canonical textual form. Further, the normalizing of the candidate ingredient data, the candidate nutritional data, and the candidate preparation instruction data may further include standardizing an ingredient unit in the candidate ingredient data to a canonical unit. Further, the normalizing of the candidate ingredient data, the candidate nutritional data, and the candidate preparation instruction data may further include ordering the candidate ingredient data in a deterministic sequence prior to the forming of the deterministic content string. Further, the normalizing of the candidate ingredient data, the candidate nutritional data, and the candidate preparation instruction data is further based on the ordering of the candidate ingredient data in the deterministic sequence prior to forming the deterministic content string.

In some embodiments, the comparing of the content-based meal fingerprint data may include retrieving the plurality of historical content-based meal fingerprint data associated with the user, within the predefined temporal lookback window. Further, the comparing of the content-based meal fingerprint data may further include loading the plurality of historical content-based meal fingerprint data into a hash-based lookup structure. Further, the comparing of the content-based meal fingerprint data may further include evaluating a membership of the content-based meal fingerprint data in the hash-based lookup structure. Further, the comparing of the content-based meal fingerprint data is further based on the evaluating of the membership of the content-based meal fingerprint data in the hash-based lookup structure.

In some embodiments, the generating of the replacement candidate meal data may include identifying an ingredient combination associated with the content-based meal fingerprint data for which duplication is identified based on the comparing of the content-based meal fingerprint data with the plurality of historical content-based meal fingerprint data in the user meal history data within the predefined temporal lookback window. Further, the generating of the replacement candidate meal data may further include modifying the at least one constraint to exclude the ingredient combination. Further, the generating of the replacement candidate meal data may further include obtaining at least one modified constraint based on the modifying of the at least one constraint. Further, the replacement candidate meal data is further generated using the at least one modified constraint.

In some embodiments, the storing of the plurality of non-duplicate validated meal data may include associating the plurality of non-duplicate validated meal data with a user identifier of the user. Further, the storing of the plurality of non-duplicate validated meal data may further include generating a timestamp corresponding to a generation time of the plurality of non-duplicate validated meal data. Further, the storing of the plurality of non-duplicate validated meal data may further include persisting the plurality of non-duplicate validated meal data, the plurality of content-based meal fingerprint data, and the timestamp in the user meal history data.

In some embodiments, the comparing of the content-based meal fingerprint data may include restricting the comparing to the plurality of historical content-based meal fingerprint data associated with a user identifier of the user. Further, the comparing of the content-based meal fingerprint data may further include excluding a historical content-based meal fingerprint data associated with a different user identifier from the plurality of historical content-based meal fingerprint data. Further, the comparing of the content-based meal fingerprint data may further include determining a duplication solely within a user-scoped data set.

In some embodiments, the forming of the respective deterministic content string from the normalized data of the non-duplicate validated meal data may include, for each non-duplicate validated meal data of the plurality of non-duplicate validated meal data normalizing a non-duplicate validated ingredient data, a non-duplicate validated nutritional data, and a non-duplicate validated preparation instruction data of the non-duplicate validated meal data to obtain a normalized non-duplicate validated ingredient data, a normalized non-duplicate validated nutritional data, and a normalized non-duplicate validated preparation instruction data. Further, the forming of the respective deterministic content string from the normalized data of the non-duplicate validated meal data may further include, for each non-duplicate validated meal data of the plurality of non-duplicate validated meal data forming the respective deterministic content string based on the normalized non-duplicate validated ingredient data, the normalized non-duplicate validated nutritional data, and the normalized non-duplicate validated preparation instruction data.

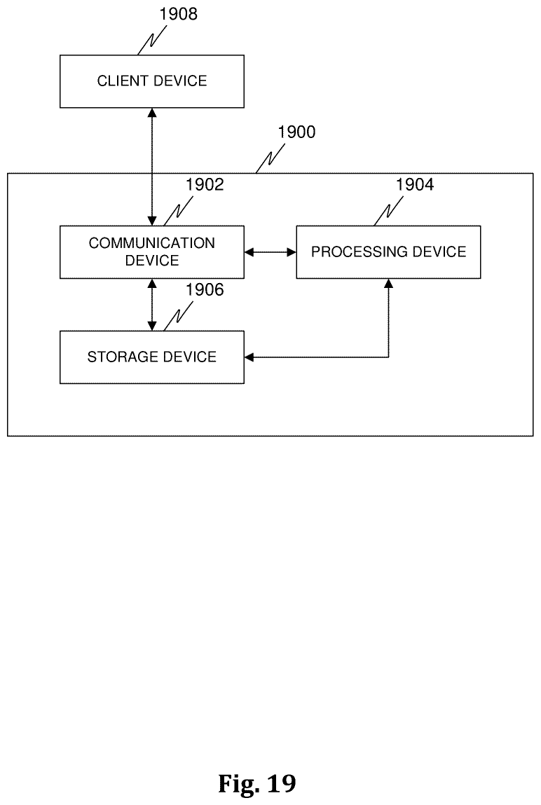

FIG. 19 illustrates a block diagram of a system 1900 of facilitating culturally-authentic and non-repetitive meal planning, in accordance with some embodiments. Accordingly, the system 1900 may include a communication device 1902 configured for receiving a meal planning request data associated with a user from a client device 1908. Further, the meal planning request data specifies a geographic sub-regional cuisine identity and a multi-day planning duration. Further, the communication device 1902 may be configured for receiving a user meal history data associated with the user from the client device. Further, the system 1900 may include a processing device 1904 communicatively coupled with the communication device 1902. Further, the processing device 1904 may be configured for identifying a candidate meal data set based on the meal planning request data. Further, the processing device 1904 may be configured for selecting a candidate meal data from the candidate meal data set. Further, the candidate meal data comprises a candidate ingredient data, a candidate nutritional data, and a candidate preparation instruction data. Further, the processing device 1904 may be configured for validating the candidate meal data against a cuisine signature data corresponding to the geographic sub-regional cuisine identity by executing a tri-level validation. Further, the tri-level validation may include determining an absence of each of one or more prohibited ingredients in the candidate ingredient data. Further, the one or more prohibited ingredients are defined by the cuisine signature data. Further, the tri-level validation may include determining a presence of each of one or more mandatory ingredients in the candidate ingredient data. Further, the one or more mandatory ingredients are defined by the cuisine signature data. Further, the tri-level validation may include calculating a cuisine signature score based on a weighted characteristic ingredient data derived from the candidate ingredient data and comparing the cuisine signature score to a configurable authenticity threshold. Further, the processing device 1904 may be configured for rejecting the candidate meal data when the tri-level validation fails. Further, the tri-level validation fails when at least one of the one or more prohibited ingredients is determined to be present in the candidate ingredient data, when at least one of the one or more mandatory ingredients is determined to be absent from the candidate ingredient data, or when the cuisine signature score is determined to be below the configurable authenticity threshold. Further, the processing device 1904 may be configured for classifying the candidate meal data as an authentic candidate meal data when the tri-level validation passes. Further, the processing device 1904 may be configured for repeating the selecting, the validating, the rejecting, and the classifying for each day of the multi-day planning duration to obtain a plurality of authentic candidate meal data, each of the plurality of authentic candidate meal data being associated with a respective day of the multi-day planning duration.

Further, in some embodiments, the processing device 1904 may be configured for projecting an ingredient usage across the multi-day planning duration by aggregating an ingredient requirement data across the plurality of authentic candidate meal data. Further, the processing device 1904 may be configured for generating an ingredient batch data representing a batch quantity for an ingredient identified in the ingredient requirement data. Further, the processing device 1904 may be configured for assigning an ingredient chain identifier to the ingredient batch data. Further, the processing device 1904 may be configured for determining a remaining quantity value associated with the ingredient chain identifier based on a consumption quantity for a day of the multi-day planning duration. Further, the processing device 1904 may be configured for rebalancing a nutritional target across remaining days of the multi-day planning duration when the remaining quantity value causes a nutritional deviation relative to the nutritional target. Further, the system 1900 may include a storage device 1906 communicatively coupled with the processing device 1904. Further, the storage device 1906 may be configured for storing the ingredient batch data in association with the ingredient chain identifier.

Further, in an embodiment, the processing device 1904 may be configured for determining a predefined temporal lookback window representing a time interval preceding receipt of the meal planning request data. Further, the processing device 1904 may be configured for generating a non-duplicate validated meal data for each authentic candidate meal data of the plurality of authentic candidate meal data by executing: normalizing the candidate ingredient data, the candidate nutritional data, and the candidate preparation instruction data of the authentic candidate meal data to obtain a normalized candidate ingredient data, a normalized candidate nutritional data, and a normalized candidate preparation instruction data; forming a deterministic content string based on the normalized candidate ingredient data, the normalized candidate nutritional data, and the normalized candidate preparation instruction data; computing a content-based meal fingerprint data for the authentic candidate meal data by applying a deterministic hash function to the deterministic content string; comparing the content-based meal fingerprint data with a plurality of historical content-based meal fingerprint data in the user meal history data within the predefined temporal lookback window to identify duplication; and generating the non-duplicate validated meal data. Further, the non-duplicate validated meal data is the authentic candidate meal data when duplication is not identified. Further, the non-duplicate validated meal data is a replacement candidate meal data when duplication is identified. Further, the replacement candidate meal data is generated based on at least one constraint defined to cause the replacement candidate meal data to satisfy the tri-level validation and to avoid duplication within the predefined temporal lookback window. Further, the processing device 1904 may be configured for aggregating the non-duplicate validated meal data generated for the plurality of authentic candidate meal data to obtain the plurality of non-duplicate validated meal data. Further, the processing device 1904 may be configured for recomputing a plurality of content-based meal fingerprint data for the plurality of non-duplicate validated meal data by forming a respective deterministic content string from a normalized data of the non-duplicate validated meal data, and applying the deterministic hash function to the respective deterministic content string for each non-duplicate validated meal data of the plurality of non-duplicate validated meal data. Further, the storage device 1906 may be configured for storing the plurality of non-duplicate validated meal data in association with the user, and the plurality of content-based meal fingerprint data in association with the user in the user meal history data. Further, the communication device 1902 may be configured for transmitting a meal plan data comprising a plurality of non-duplicate validated meal data to the client device 1908 associated with the user.

Further, in some embodiments, the calculating of the cuisine signature score may include normalizing the candidate ingredient data to obtain a canonical ingredient name data. Further, the calculating of the cuisine signature score may further include normalizing the candidate ingredient data to obtain a canonical ingredient quantity data. Further, the calculating of the cuisine signature score may further include constructing a canonical ingredient vector based on the canonical ingredient name data and the canonical ingredient quantity data. Further, the calculating of the cuisine signature score may further include weighting the canonical ingredient vector using a cuisine-specific weight parameter defined in the cuisine signature data to obtain a weighted canonical ingredient vector. Further, the calculating of the cuisine signature score may further include computing the cuisine signature score based on the weighted canonical ingredient vector.

Further, in some embodiments, the comparing of the cuisine signature score to the configurable authenticity threshold may include selecting the configurable authenticity threshold based on a geographic specificity level associated with the geographic sub-regional cuisine identity. Further, the comparing of the cuisine signature score to the configurable authenticity threshold may further include determining a threshold satisfaction condition based on the cuisine signature score and the configurable authenticity threshold. Further, the comparing of the cuisine signature score to the configurable authenticity threshold may further include determining an authenticity result data representing whether the threshold satisfaction condition is satisfied or not

Further, in some embodiments, the projecting of the ingredient usage may include aggregating an ingredient quantity data across the multi-day planning duration. Further, the projecting of the ingredient usage may further include generating the ingredient requirement data based on the ingredient quantity data.

Further, in some embodiments, the generating of the ingredient batch data may include determining a batch purchase quantity for the ingredient based on the ingredient requirement data. Further, the generating of the ingredient batch data may further include initializing the batch quantity in the ingredient batch data with an initial value based on the batch purchase quantity. Further, the generating of the ingredient batch data is further based on the initializing. Further, the ingredient batch data is generated prior to assigning the ingredient chain identifier.

Further, in some embodiments, the determining of the remaining quantity value may include retrieving the ingredient batch data associated with the ingredient chain identifier. Further, the determining of the remaining quantity value may further include computing the remaining quantity value by subtracting the consumption quantity from the initial value of the batch quantity in the ingredient batch data.

In some embodiments, the present disclosure provides a non-transitory computer-readable medium storing instructions that, when executed by one or more processors of an online server, cause the online server to perform operations for facilitating culturally-authentic and non-repetitive meal planning. Further, the operations may include receiving a meal planning request data associated with a user from a client device. Further, the meal planning request data specifies a geographic sub-regional cuisine identity and a multi-day planning duration. Further, the operations may include receiving a user meal history data associated with the user from the client device 1908. Further, the operations may include identifying a candidate meal data set based on the meal planning request data. Further, the operations may include selecting a candidate meal data from the candidate meal data set. Further, the candidate meal data comprises a candidate ingredient data, a candidate nutritional data, and a candidate preparation instruction data. Further, the operations may include validating the candidate meal data against a cuisine signature data corresponding to the geographic sub-regional cuisine identity by executing a tri-level validation. Further, the tri-level validation may include determining an absence of each of one or more prohibited ingredients in the candidate ingredient data. Further, the one or more prohibited ingredients are defined by the cuisine signature data. Further, the tri-level validation may include determining a presence of each of one or more mandatory ingredients in the candidate ingredient data. Further, the one or more mandatory ingredients are defined by the cuisine signature data. Further, the tri-level validation may include calculating a cuisine signature score based on a weighted characteristic ingredient data derived from the candidate ingredient data and comparing the cuisine signature score to a configurable authenticity threshold. Further, the operations may include rejecting the candidate meal data when the tri-level validation fails. Further, the tri-level validation fails when at least one of the one or more prohibited ingredients is determined to be present in the candidate ingredient data, when at least one of the one or more mandatory ingredients is determined to be absent from the candidate ingredient data, or when the cuisine signature score is determined to be below the configurable authenticity threshold. Further, the operations may include classifying the candidate meal data as an authentic candidate meal data when the tri-level validation passes. Further, the operations may include repeating the selecting, the validating, the rejecting, and the classifying for each day of the multi-day planning duration to obtain a plurality of authentic candidate meal data, each of the plurality of authentic candidate meal data being associated with a respective day of the multi-day planning duration. Further, the operations may include projecting an ingredient usage across the multi-day planning duration by aggregating an ingredient requirement data across the plurality of authentic candidate meal data. Further, the operations may include generating an ingredient batch data representing a batch quantity for an ingredient identified in the ingredient requirement data. Further, the operations may include assigning an ingredient chain identifier to the ingredient batch data. Further, the operations may include storing the ingredient batch data in association with the ingredient chain identifier. Further, the operations may include determining a remaining quantity value associated with the ingredient chain identifier based on a consumption quantity for a day of the multi-day planning duration. Further, the operations may include rebalancing a nutritional target across remaining days of the multi-day planning duration when the remaining quantity value causes a nutritional deviation relative to the nutritional target. Further, the operations may include determining a predefined temporal lookback window representing a time interval preceding receipt of the meal planning request data. Further, the operations may include generating a non-duplicate validated meal data for each authentic candidate meal data of the plurality of authentic candidate meal data by executing: normalizing the candidate ingredient data, the candidate nutritional data, and the candidate preparation instruction data of the authentic candidate meal data to obtain a normalized candidate ingredient data, a normalized candidate nutritional data, and a normalized candidate preparation instruction data; forming a deterministic content string based on the normalized candidate ingredient data, the normalized candidate nutritional data, and the normalized candidate preparation instruction data; computing a content-based meal fingerprint data for the authentic candidate meal data by applying a deterministic hash function to the deterministic content string; comparing the content-based meal fingerprint data with a plurality of historical content-based meal fingerprint data in the user meal history data within the predefined temporal lookback window to identify duplication; and generating the non-duplicate validated meal data. Further, the non-duplicate validated meal data is the authentic candidate meal data when duplication is not identified. Further, the non-duplicate validated meal data is a replacement candidate meal data when duplication is identified. Further, the replacement candidate meal data is generated based on at least one constraint defined to cause the replacement candidate meal data to satisfy the tri-level validation and to avoid duplication within the predefined temporal lookback window. Further, the operations may include aggregating the non-duplicate validated meal data to obtain a plurality of non-duplicate validated meal data. Further, the operations may include recomputing a plurality of content-based meal fingerprint data for the plurality of non-duplicate validated meal data by forming a respective deterministic content string from a normalized data of the non-duplicate validated meal data, and applying the deterministic hash function to the respective deterministic content string for each non-duplicate validated meal data of the plurality of non-duplicate validated meal data. Further, the operations may include storing the plurality of non-duplicate validated meal data in association with the user, and the plurality of content-based meal fingerprint data in association with the user in the user meal history data. Further, the operations may include transmitting a meal plan data comprising the plurality of non-duplicate validated meal data to the client device 1908 associated with the user.

Further, in some embodiments, the rebalancing of the nutritional target may include calculating a cumulative nutritional deviation attributable to the remaining quantity value. Further, the rebalancing of the nutritional target may further include determining a number of the remaining days in the multi-day planning duration. Further, the rebalancing of the nutritional target may further include computing a per-day nutritional adjustment value based on the cumulative nutritional deviation and the number of the remaining days. Further, the rebalancing of the nutritional target may further include updating a daily nutritional target for each of the number of the remaining days using the per-day nutritional adjustment value. Further, the rebalancing of the nutritional target is further based on the updating of the daily nutritional target.

Further, in some embodiments, the normalizing of the candidate ingredient data, the candidate nutritional data, and the candidate preparation instruction data may include converting an ingredient name in the candidate ingredient data to a canonical textual form. Further, the normalizing of the candidate ingredient data, the candidate nutritional data, and the candidate preparation instruction data may further include standardizing an ingredient unit in the candidate ingredient data to a canonical unit. Further, the normalizing of the candidate ingredient data, the candidate nutritional data, and the candidate preparation instruction data may further include ordering the candidate ingredient data in a deterministic sequence prior to the forming of the deterministic content string. Further, the normalizing of the candidate ingredient data, the candidate nutritional data, and the candidate preparation instruction data is further based on the ordering of the candidate ingredient data in the deterministic sequence prior to forming the deterministic content string.

Further, in some embodiments, the comparing of the content-based meal fingerprint data may include retrieving the plurality of historical content-based meal fingerprint data associated with the user, within the predefined temporal lookback window. Further, the comparing of the content-based meal fingerprint data may further include loading the plurality of historical content-based meal fingerprint data into a hash-based lookup structure. Further, the comparing of the content-based meal fingerprint data may further include evaluating a membership of the content-based meal fingerprint data in the hash-based lookup structure. Further, the comparing of the content-based meal fingerprint data is further based on the evaluating of the membership of the content-based meal fingerprint data in the hash-based lookup structure.

Further, in some embodiments, the generating of the replacement candidate meal data may include identifying an ingredient combination associated with the content-based meal fingerprint data for which duplication is identified based on the comparing of the content-based meal fingerprint data with the plurality of historical content-based meal fingerprint data in the user meal history data within the predefined temporal lookback window. Further, the generating of the replacement candidate meal data may further include modifying the at least one constraint to exclude the ingredient combination. Further, the generating of the replacement candidate meal data may further include obtaining at least one modified constraint based on the modifying of the at least one constraint. Further, the replacement candidate meal data is further generated using the at least one modified constraint.

Further, in some embodiments, the storing of the plurality of non-duplicate validated meal data may include associating the plurality of non-duplicate validated meal data with a user identifier of the user. Further, the storing of the plurality of non-duplicate validated meal data may further include generating a timestamp corresponding to a generation time of the plurality of non-duplicate validated meal data. Further, the storing of the plurality of non-duplicate validated meal data may further include persisting the plurality of non-duplicate validated meal data, the plurality of content-based meal fingerprint data, and the timestamp in the user meal history data.

Further, in some embodiments, the comparing of the content-based meal fingerprint data may include restricting the comparing to the plurality of historical content-based meal fingerprint data associated with a user identifier of the user. Further, the comparing of the content-based meal fingerprint data may further include excluding a historical content-based meal fingerprint data associated with a different user identifier from the plurality of historical content-based meal fingerprint data. Further, the comparing of the content-based meal fingerprint data may further include determining a duplication solely within a user-scoped data set.

Further, in some embodiments, the forming of the respective deterministic content string from the normalized data of the non-duplicate validated meal data may include, for each non-duplicate validated meal data of the plurality of non-duplicate validated meal data normalizing a non-duplicate validated ingredient data, a non-duplicate validated nutritional data, and a non-duplicate validated preparation instruction data of the non-duplicate validated meal data to obtain a normalized non-duplicate validated ingredient data, a normalized non-duplicate validated nutritional data, and a normalized non-duplicate validated preparation instruction data. Further, the forming of the respective deterministic content string from the normalized data of the non-duplicate validated meal data may further include, for each non-duplicate validated meal data of the plurality of non-duplicate validated meal data forming the respective deterministic content string based on the normalized non-duplicate validated ingredient data, the normalized non-duplicate validated nutritional data, and the normalized non-duplicate validated preparation instruction data.

Although the invention has been explained in relation to its preferred embodiment, it is to be understood that many other possible modifications and variations can be made without departing from the spirit and scope of the invention as hereinafter claimed.

What is claimed is:

1. A method of facilitating culturally-authentic and non-repetitive meal planning, the method comprising:
receiving, using a communication device, a meal planning request data associated with a user from a client device, wherein the meal planning request data specifies a geographic sub-regional cuisine identity and a multi-day planning duration;
receiving, using the communication device, a user meal history data associated with the user from the client device;
identifying, using a processing device, a candidate meal data set based on the meal planning request data;
selecting, using the processing device, a candidate meal data from the candidate meal data set, wherein the candidate meal data comprises a candidate ingredient data, a candidate nutritional data, and a candidate preparation instruction data;
validating, using the processing device, the candidate meal data against a cuisine signature data corresponding to the geographic sub-regional cuisine identity by executing a tri-level validation comprising:
determining an absence of each of one or more prohibited ingredients in the candidate ingredient data, wherein the one or more prohibited ingredients are defined by the cuisine signature data;
determining a presence of each of one or more mandatory ingredients in the candidate ingredient data, wherein the one or more mandatory ingredients are defined by the cuisine signature data; and
calculating a cuisine signature score based on a weighted characteristic ingredient data derived from the candidate ingredient data and comparing the cuisine signature score to a configurable authenticity threshold;
rejecting, using the processing device, the candidate meal data when the tri-level validation fails, wherein the tri-level validation fails when at least one of the one or more prohibited ingredients is determined to be present in the candidate ingredient data, when at least one of the one or more mandatory ingredients is determined to be absent from the candidate ingredient data, or when the cuisine signature score is determined to be below the configurable authenticity threshold;
classifying, using the processing device, the candidate meal data as an authentic candidate meal data when the tri-level validation passes; and
repeating, using the processing device, the selecting, the validating, the rejecting, and the classifying for each day of the multi-day planning duration to obtain a plurality of authentic candidate meal data, each of the plurality of authentic candidate meal data being associated with a respective day of the multi-day planning duration.

2. The method of claim 1 further comprising:
projecting, using the processing device, an ingredient usage across the multi-day planning duration by aggregating an ingredient requirement data across the plurality of authentic candidate meal data;
generating, using the processing device, an ingredient batch data representing a batch quantity for an ingredient identified in the ingredient requirement data;
assigning, using the processing device, an ingredient chain identifier to the ingredient batch data;
storing, using a storage device, the ingredient batch data in association with the ingredient chain identifier;
determining, using the processing device, a remaining quantity value associated with the ingredient chain identifier based on a consumption quantity for a day of the multi-day planning duration; and
rebalancing, using the processing device, a nutritional target across remaining days of the multi-day planning duration when the remaining quantity value causes a nutritional deviation relative to the nutritional target.

3. The method of claim 1 further comprising:
determining, using the processing device, a predefined temporal lookback window representing a time interval preceding receipt of the meal planning request data;
generating, using the processing device, a non-duplicate validated meal data for each authentic candidate meal data of the plurality of authentic candidate meal data by executing:
normalizing the candidate ingredient data, the candidate nutritional data, and the candidate preparation instruction data of the authentic candidate meal data to obtain a normalized candidate ingredient data, a normalized candidate nutritional data, and a normalized candidate preparation instruction data;
forming a deterministic content string based on the normalized candidate ingredient data, the normalized candidate nutritional data, and the normalized candidate preparation instruction data;
computing a content-based meal fingerprint data for the authentic candidate meal data by applying a deterministic hash function to the deterministic content string;
comparing the content-based meal fingerprint data with a plurality of historical content-based meal fingerprint data in the user meal history data within the predefined temporal lookback window to identify duplication; and
generating the non-duplicate validated meal data, wherein the non-duplicate validated meal data is the authentic candidate meal data when duplication is not identified, wherein the non-duplicate validated meal data is a replacement candidate meal data when duplication is identified, wherein the replacement candidate meal data is generated based on at least one constraint defined to cause the replacement candidate meal data to satisfy the tri-level validation and to avoid duplication within the predefined temporal lookback window;
aggregating, using the processing device, the non-duplicate validated meal data to obtain a plurality of non-duplicate validated meal data;
recomputing, using the processing device, a plurality of content-based meal fingerprint data for the plurality of non-duplicate validated meal data by forming a respective deterministic content string from a normalized data of the non-duplicate validated meal data, and applying the deterministic hash function to the respective deterministic content string for each non-duplicate validated meal data of the plurality of non-duplicate validated meal data;
storing, using the storage device, the plurality of non-duplicate validated meal data in association with the user, and the plurality of content-based meal fingerprint data in association with the user in the user meal history data; and
transmitting, using the communication device, a meal plan data comprising the plurality of non-duplicate validated meal data to the client device associated with the user.

4. The method of claim 1, wherein the calculating of the cuisine signature score further comprises:
normalizing the candidate ingredient data to obtain a canonical ingredient name data;
normalizing the candidate ingredient data to obtain a canonical ingredient quantity data;
constructing a canonical ingredient vector based on the canonical ingredient name data and the canonical ingredient quantity data;
weighting the canonical ingredient vector using a cuisine-specific weight parameter defined in the cuisine signature data to obtain a weighted canonical ingredient vector; and
computing the cuisine signature score based on the weighted canonical ingredient vector.

5. The method of claim 1, wherein the comparing of the cuisine signature score to the configurable authenticity threshold further comprises:
selecting the configurable authenticity threshold based on a geographic specificity level associated with the geographic sub-regional cuisine identity;
determining a threshold satisfaction condition based on the cuisine signature score and the configurable authenticity threshold; and
determining an authenticity result data representing whether the threshold satisfaction condition is satisfied or not.

6. The method of claim 2, wherein the projecting of the ingredient usage further comprises:
aggregating an ingredient quantity data across the multi-day planning duration; and
generating the ingredient requirement data based on the ingredient quantity data.

7. The method of claim 2, wherein the generating of the ingredient batch data further comprises:
determining a batch purchase quantity for the ingredient based on the ingredient requirement data; and
initializing the batch quantity in the ingredient batch data with an initial value based on the batch purchase quantity, wherein the generating of the ingredient batch data is further based on the initializing, wherein the ingredient batch data is generated prior to assigning the ingredient chain identifier.

8. The method of claim 7, wherein the determining of the remaining quantity value further comprises:
retrieving the ingredient batch data associated with the ingredient chain identifier; and
computing the remaining quantity value by subtracting the consumption quantity from the initial value of the batch quantity in the ingredient batch data.

9. The method of claim 2, wherein the rebalancing of the nutritional target further comprises:
calculating a cumulative nutritional deviation attributable to the remaining quantity value;
determining a number of the remaining days in the multi-day planning duration;
computing a per-day nutritional adjustment value based on the cumulative nutritional deviation and the number of the remaining days; and
updating a daily nutritional target for each of the number of the remaining days using the per-day nutritional adjustment value, wherein the rebalancing of the nutritional target is further based on the updating of the daily nutritional target.

10. The method of claim 3, wherein the normalizing of the candidate ingredient data, the candidate nutritional data, and the candidate preparation instruction data further comprises:
converting an ingredient name in the candidate ingredient data to a canonical textual form;
standardizing an ingredient unit in the candidate ingredient data to a canonical unit; and
ordering the candidate ingredient data in a deterministic sequence prior to the forming of the deterministic content string, wherein the normalizing of the candidate ingredient data, the candidate nutritional data, and the candidate preparation instruction data is further based on the ordering of the candidate ingredient data in the deterministic sequence prior to forming the deterministic content string.

11. The method of claim 3, wherein the comparing of the content-based meal fingerprint data further comprises:
retrieving the plurality of historical content-based meal fingerprint data associated with the user, within the predefined temporal lookback window;
loading the plurality of historical content-based meal fingerprint data into a hash-based lookup structure; and
evaluating a membership of the content-based meal fingerprint data in the hash-based lookup structure, wherein the comparing of the content-based meal fingerprint data is further based on the evaluating of the membership of the content-based meal fingerprint data in the hash-based lookup structure.

12. The method of claim 3, wherein the generating of the replacement candidate meal data further comprises:
identifying an ingredient combination associated with the content-based meal fingerprint data for which duplication is identified based on the comparing of the content-based meal fingerprint data with the plurality of historical content-based meal fingerprint data in the user meal history data within the predefined temporal lookback window;
modifying the at least one constraint to exclude the ingredient combination; and
obtaining at least one modified constraint based on the modifying of the at least one constraint, wherein the replacement candidate meal data is further generated using the at least one modified constraint.

13. The method of claim 3, wherein the storing of the plurality of non-duplicate validated meal data further comprises:
associating the plurality of non-duplicate validated meal data with a user identifier of the user;
generating a timestamp corresponding to a generation time of the plurality of non-duplicate validated meal data; and
persisting the plurality of non-duplicate validated meal data, the plurality of content-based meal fingerprint data, and the timestamp in the user meal history data.

14. The method of claim 3, wherein the comparing of the content-based meal fingerprint data further comprises:
restricting the comparing to the plurality of historical content-based meal fingerprint data associated with a user identifier of the user;
excluding a historical content-based meal fingerprint data associated with a different user identifier from the plurality of historical content-based meal fingerprint data; and
determining a duplication solely within a user-scoped data set.

15. The method of claim 3, wherein the forming of the respective deterministic content string from the normalized data of the non-duplicate validated meal data further comprises, for each non-duplicate validated meal data of the plurality of non-duplicate validated meal data:
normalizing a non-duplicate validated ingredient data, a non-duplicate validated nutritional data, and a non-duplicate validated preparation instruction data of the non-duplicate validated meal data to obtain a normalized non-duplicate validated ingredient data, a normalized non-duplicate validated nutritional data, and a normalized non-duplicate validated preparation instruction data; and
forming the respective deterministic content string based on the normalized non-duplicate validated ingredient data, the normalized non-duplicate validated nutritional data, and the normalized non-duplicate validated preparation instruction data.

16. A system of facilitating culturally-authentic and non-repetitive meal planning, the system comprising:
a communication device configured for:
receiving a meal planning request data associated with a user from a client device, wherein the meal planning request data specifies a geographic sub-regional cuisine identity and a multi-day planning duration; and
receiving a user meal history data associated with the user from the client device; and
a processing device communicatively coupled with the communication device, wherein the processing device is configured for:
identifying a candidate meal data set based on the meal planning request data;
selecting a candidate meal data from the candidate meal data set, wherein the candidate meal data comprises a candidate ingredient data, a candidate nutritional data, and a candidate preparation instruction data;
validating the candidate meal data against a cuisine signature data corresponding to the geographic sub-regional cuisine identity by executing a tri-level validation comprising:
determining an absence of each of one or more prohibited ingredients in the candidate ingredient data, wherein the one or more prohibited ingredients are defined by the cuisine signature data,
determining a presence of each of one or more mandatory ingredients in the candidate ingredient data, wherein the one or more mandatory ingredients are defined by the cuisine signature data, and
calculating a cuisine signature score based on a weighted characteristic ingredient data derived from the candidate ingredient data and comparing the cuisine signature score to a configurable authenticity threshold;
rejecting the candidate meal data when the tri-level validation fails, wherein the tri-level validation fails when at least one of the one or more prohibited ingredients is determined to be present in the candidate ingredient data, when at least one of the one or more mandatory ingredients is determined to be absent from the candidate ingredient data, or when the cuisine signature score is determined to be below the configurable authenticity threshold;
classifying the candidate meal data as an authentic candidate meal data when the tri-level validation passes; and
repeating the selecting, the validating, the rejecting, and the classifying for each day of the multi-day planning duration to obtain a plurality of authentic candidate meal data, each of the plurality of authentic candidate meal data being associated with a respective day of the multi-day planning duration.

17. The system of claim 16, wherein the processing device is further configured for:
projecting an ingredient usage across the multi-day planning duration by aggregating an ingredient requirement data across the plurality of authentic candidate meal data;
generating an ingredient batch data representing a batch quantity for an ingredient identified in the ingredient requirement data;
assigning an ingredient chain identifier to the ingredient batch data;
determining a remaining quantity value associated with the ingredient chain identifier based on a consumption quantity for a day of the multi-day planning duration; and
rebalancing a nutritional target across remaining days of the multi-day planning duration when the remaining quantity value causes a nutritional deviation relative to the nutritional target, wherein the system further comprises a storage device communicatively coupled with the processing device, wherein the storage device is configured for storing the ingredient batch data in association with the ingredient chain identifier.

18. The system of claim 16, wherein the processing device is further configured for:
determining a predefined temporal lookback window representing a time interval preceding receipt of the meal planning request data;
generating a non-duplicate validated meal data for each authentic candidate meal data of the plurality of authentic candidate meal data by executing:
normalizing the candidate ingredient data, the candidate nutritional data, and the candidate preparation instruction data of the authentic candidate meal data to obtain a normalized candidate ingredient data, a normalized candidate nutritional data, and a normalized candidate preparation instruction data;
forming a deterministic content string based on the normalized candidate ingredient data, the normalized candidate nutritional data, and the normalized candidate preparation instruction data;
computing a content-based meal fingerprint data for the authentic candidate meal data by applying a deterministic hash function to the deterministic content string;
comparing the content-based meal fingerprint data with a plurality of historical content-based meal fingerprint data in the user meal history data within the predefined temporal lookback window to identify duplication; and
generating the non-duplicate validated meal data, wherein the non-duplicate validated meal data is the authentic candidate meal data when duplication is not identified, wherein the non-duplicate validated meal data is a replacement candidate meal data when duplication is identified, wherein the replacement candidate meal data is generated based on at least one constraint defined to cause the replacement candidate meal data to satisfy the tri-level validation and to avoid duplication within the predefined temporal lookback window;
aggregating the non-duplicate validated meal data generated for the plurality of authentic candidate meal data to obtain the plurality of non-duplicate validated meal data; and
recomputing a plurality of content-based meal fingerprint data for the plurality of non-duplicate validated meal data by forming a respective deterministic content string from a normalized data of the non-duplicate validated meal data, and applying the deterministic hash function to the respective deterministic content string for each non-duplicate validated meal data of the plurality of non-duplicate validated meal data, wherein the storage device is further configured for storing the plurality of non-duplicate validated meal data in association with the user, and the plurality of content-based meal fingerprint data in association with the user in the user meal history data, wherein the communication device is further configured for transmitting a meal plan data comprising the plurality of non-duplicate validated meal data to the client device associated with the user.

19. The system of claim 16, wherein the calculating of the cuisine signature score further comprises:
normalizing the candidate ingredient data to obtain a canonical ingredient name data;
normalizing the candidate ingredient data to obtain a canonical ingredient quantity data;
constructing a canonical ingredient vector based on the canonical ingredient name data and the canonical ingredient quantity data;
weighting the canonical ingredient vector using a cuisine-specific weight parameter defined in the cuisine signature data to obtain a weighted canonical ingredient vector; and
computing the cuisine signature score based on the weighted canonical ingredient vector.

20. A non-transitory computer-readable medium storing instructions that, when executed by one or more processors of an online server, cause the online server to perform operations for facilitating culturally-authentic and non-repetitive meal planning, the operations comprising:
receiving a meal planning request data associated with a user from a client device, wherein the meal planning request data specifies a geographic sub-regional cuisine identity and a multi-day planning duration;
receiving a user meal history data associated with the user from the client device;
identifying a candidate meal data set based on the meal planning request data;
selecting a candidate meal data from the candidate meal data set, wherein the candidate meal data comprises a candidate ingredient data, a candidate nutritional data, and a candidate preparation instruction data;
validating the candidate meal data against a cuisine signature data corresponding to the geographic sub-regional cuisine identity by executing a tri-level validation comprising:
determining an absence of each of one or more prohibited ingredients in the candidate ingredient data, wherein the one or more prohibited ingredients are defined by the cuisine signature data;
determining a presence of each of one or more mandatory ingredients in the candidate ingredient data, wherein the one or more mandatory ingredients are defined by the cuisine signature data; and
calculating a cuisine signature score based on a weighted characteristic ingredient data derived from the candidate ingredient data and comparing the cuisine signature score to a configurable authenticity threshold;
rejecting the candidate meal data when the tri-level validation fails, wherein the tri-level validation fails when at least one of the one or more prohibited ingredients is determined to be present in the candidate ingredient data, when at least one of the one or more mandatory ingredients is determined to be absent from the candidate ingredient data, or when the cuisine signature score is determined to be below the configurable authenticity threshold;
classifying the candidate meal data as an authentic candidate meal data when the tri-level validation passes; and
repeating the selecting, the validating, the rejecting, and the classifying for each day of the multi-day planning duration to obtain a plurality of authentic candidate meal data, each of the plurality of authentic candidate meal data being associated with a respective day of the multi-day planning duration.
